SEQUENCE LISTING (73 sequences)

SEQ ID NO: 1 - DNA, 1755 bp, Artificial Sequence
  Synthetic sequence. ShTnsB_pCO1
atgaacagtcaacagaacccagacctcgctgttcatcccttagcaattcctatggagggcctacttggcgaatccgcgactacattggagaagaatgttattgcaacacaactttctgaggaagctcaagtgaagctcgaagtcattcaaagtctcctggaaccgtgtgaccgaacgacttacggacaaaagctcagggaggcggccgaaaagttgaatgtttcgctccggacggttcaaaggctcgtcaagaattgggaacaagatggtctagttggattaacacaaacctcccgagccgacaagggcaaacaccgaattggcgaattctgggagaactttatcacaaagacctacaaggaaggcaacaagggttctaagcgtatgactcctaaacaagttgcgctcagagtcgaggctaaagctcgtgaactcaaagactcaaaaccaccgaactacaaaacagttctgagagttcttgcaccgattcttgaaaagcaacaaaaggctaagtcgatccgctctcctggctggcgcggaacaaccctttctgtcaaaacacgcgaaggcaaggatcttagtgtggactacagtaatcacgtctggcaatgcgaccacacgagagtagatgtgttgctggtagatcaacatggagaaattctatcacgtccgtggcttacaactgtaattgacacttactcacgctgcataatggggataaacttgggattcgacgcaccgagttccggcgttgtcgcacttgctcttagacatgctatccttcctaaacgctacggcagcgagtacaaactacattgtgaatggggaacttacggaaagccagagcacttttacacagatggaggcaaagacttccgctcaaatcacctcagccaaattggtgctcaattgggtttcgtctgccatctacgtgaccgtccaagtgaaggtggagtggtcgaacgaccattcaaaactcttaacgatcagttattctcaacccttccgggatacactggttcaaatgtccaagagcgtccggaggatgctgaaaaggatgctcgtttaacacttcgagagcttgaacaactcctggtacggtacattgtggaccgctacaaccagtccattgatgcacgtatgggagaccaaacgcgctttgaaaggtgggaagcgggtctgccaacagtgcctgtaccaatccctgaacgtgacctcgacatctgtcttatgaagcaaagtaggagaacggttcaaaggggaggatgtctccagtttcaaaatcttatgtaccgaggtgaatatctcgccgggtatgctggtgaaactgtaaacttacgtttcgatccacgtgatattaccaccatcctcgtttatcgacaggaaaacaatcaggaagtgttcttaactagggctcatgcacaaggacttgaaacagaacaactagctctggacgaggcggaggcagcatctcgtcgtcttagaacggctgggaagactatcagtaatcagtcactgttacaggaagtcgttgatcgtgatgctctggtcgctaccaaaaagtctaggaaggagcgtcagaaactagaacaaacggtcctccgatctgccgctgtggacgagtcaaatcgggagtcattaccttctcaaattgtggagcccgatgaagttgaaagcacagaaacagttcatagccaatacgaggatattgaagtctgggattatgaacagctacgggaggaatacgggttctag

SEQ ID NO: 2 - DNA, 1755 bp, Artificial Sequence
  Synthetic sequence. ShTnsB_pCO2
atgaactctcaacaaaaccctgatctagctgttcatcctcttgcaataccaatggagggtctgcttggagagtctgcaactactcttgagaagaatgtaattgcaacacagctttctgaggaggcacaagttaaacttgaagtaattcagtcactgctggaaccttgtgaccgtacgacttacggacagaagttgagggaggctgccgaaaagctgaatgttagtttaagaactgtacaaaggctagtcaagaactgggaacaagatggtcttgtcgggttgacacaaacttcaagagctgacaagggcaaacaccggattggagagttctgggaaaacttcataactaaaacgtacaaggagggcaataagggttctaagcgtatgactcctaaacaagttgcacttcgagttgaagcgaaagcaagggaactgaaagatagtaaaccacctaactacaaaacggttctgagagttcttgctccgattctggaaaagcaacaaaaggccaaatctattcgatctcctggttggaggggaactactttatctgttaaaacaagggaggggaaagacctcagtgttgactattctaatcacgtatggcaatgcgatcatacaagagttgatgtactattggtagatcaacatggtgaaattctgagtaggccgtggctaactactgttattgatacttattcacggtgcataatggggattaacctggggttcgatgctccttcctccggcgtcgtagcattggcacttagacatgctattctacctaaacgctatggatcagagtacaaactacattgtgaatggggaacttatgggaagccagaacacttctataccgatggcggcaaagactttcgtagtaatcatctaagtcaaattggtgctcaattgggattcgtctgtcatcttagagatagaccaagtgaaggaggagtagttgaaagacccttcaagactcttaacgaccaattgttctcaacattacctggatatactggtagtaatgtacaagaacgacctgaggacgctgagaaggatgctagacttactctgagagagttagagcaactcttagttcgctacattgtggacagatacaatcagagtattgacgctagaatgggagaccaaacaagatttgaacgatgggaagctggcttgccaacagttccagttcctatccccgagcgcgatttggacatctgcctcatgaagcaatctaggagaactgttcaaagaggtggatgtctccaatttcagaatctaatgtacaggggtgaatatctcgctggttatgctggagagactgtgaacctcagatttgaccctagagatattactactatcttggtctatcgtcaggagaacaatcaggaggtgttcttaacaagagcacatgcacaaggacttgaaacagaacaacttgctttagatgaagcagaagcagcaagtaggagattaagaaccgctgggaagacgatttcaaaccagtcactattacaggaagtagtggaccgtgacgcactcgtggccaccaaaaaatcacggaaggaacggcagaagctcgaacaaactgtgttacgatcagccgctgtggacgagtccaatcgagagtcattaccctcacaaattgtcgagccagatgaagtagaatctactgaaacagtacattcacaatatgaggatattgaagtgtgggattatgaacaattgcgtgaggagtatgggttctaa

SEQ ID NO: 3 - DNA, 828 bp, Artificial Sequence
  Synthetic sequence. ShTnsC_pCO1
atgaccgaagctcaagctattgctaaacaactaggtggtgttaaacctgatgatgagtggctacaagcagaaattgcaaggttgaagggcaaatctatagtcccacttcaacaagttaagacacttcatgattggcttgatggaaagcgcaaggctaggaaatcatgtcgcgtggttggggaatctcggacgggcaagaccgtcgcttgtgatgcttaccgttatcgacataagccacaacaggaagcgggtcgtccaccaacagtgccagtcgtttacatccgaccacaccagaaatgcgggcctaaagacttattcaagaagattacagagtacctcaagtatcgagttacgaaaggaactgtgtcagatttccgagaccgcacaattgaagtgttaaaaggttgcggtgtggaaatgctgataattgacgaagctgacagactgaaacctgaaaccttcgctgacgttcgtgacattgccgaggacttgggaattgcagtggtcctagtcggaacagataggcttgatgcagtgattaagcgggacgaacaagtgcttgaacgcttccgcgcgcacctaaggtttgggaaattgagcggcgaggactttaagaatactgttgagatgtgggaacaaatggtcctcaaactcccggtcagtagtaatctgaaatctaaggagatgctaaggatacttacttctgctacagagggttacattggtcgcttggacgagattctacgggaggcggccatccgcagtctgtctcgtggactcaagaaaatcgacaaggcagttctccaggaggttgctaaagagtacaaa

SEQ ID NO: 4 - DNA, 831 bp, Artificial Sequence
  Synthetic sequence. ShTnsC_pCO2
atgaccgaggcgcaagctatagcaaaacaactcggaggtgtaaaaccagatgacgaatggcttcaagccgaaatcgcaagactaaagggaaaatctattgtgccattacaacaagtgaaaaccttacatgactggctcgatggaaaacgaaaagctaggaaatcatgtcgcgtggtaggagaatctcgaactgggaaaacagttgcatgtgatgcttatcgctacaggcacaagcctcaacaggaggccggtaggccacctaccgttccagtcgtgtacatccgaccacaccaaaagtgtggacctaaggacttattcaaaaagatcacggaatatcttaagtacagagttactaaaggaacagtcagtgacttccgagacagaacaattgaggttctgaaaggttgtggagttgagatgttaatcattgacgaggctgatcgacttaaacctgaaactttcgcagatgttagagatattgccgaagatttgggcattgcagtcgtgttagttggaaccgatagacttgatgcagttattaagcgtgatgaacaagtattggaacgttttagagcacaccttcgatttggcaaactctctggcgaagacttcaagaacacagttgaaatgtgggaacaaatggtactgaaattacctgtttccagtaatcttaaatctaaggaaatgctgagaatcttgaccagcgctacagaaggctacatcggaaggttggacgaaatcctccgagaagctgctatacggtctttaagtagagggctcaagaagatagacaaagctgtcttacaggaggtcgctaaggagtacaaatag

SEQ ID NO: 5 - DNA, 504 bp, Artificial Sequence
  Synthetic sequence. ShTniQ_pCO1
atgattgaagcaccagatgtaaaaccctggctcttcctcattaagccttacgaaggtgaatcactcagccacttcctcgggcggttcagacgagcaaatcatctatcagcatctggtcttggcactcttgctggaattggagctattgtggctcggtgggaacgttttcacttcaatccacgcccatctcaacaagagcttgaagctatagcatcagtggtagaggtggccgcacaaagactagctcaaatgttacctcctgctggagtcggaatgcagcatgaaccgataaggttatgcggcgcatgttatgcagaatcaccttgtcacaggatcgagtggcagtacaaatcagtatggaagtgtgatcggcatcaattgaaaatcctagctaaatgtcctaattgtcaagctcctttcaaaatgccagctctttgggaagatggatgttgccataggtgtcgaatgccattcgcagaaatggccaagttgcaaaaggtctaa

SEQ ID NO: 6 - DNA, 504 bp, Artificial Sequence
  Synthetic sequence. ShTniQ_pCO2
atgatcgaagctccggatgtaaaaccctggctcttccttattaagccttatgaaggagaatcactatctcactttctcggtcgctttcgcagagctaatcatctatctgcaagtgggctcggaaccttagctggaattggagctatcgtagctagatgggaaagatttcactttaacccaagaccttctcaacaagaattggaagctattgcatcagtagttgaagttgctgctcaaagattggctcaaatgttacctcctgctggtgttggaatgcaacatgaacctatacgtttatgtggagcttgttatgcagaatcaccttgtcatcgtattgaatggcagtacaaaagcgtctggaagtgtgaccgtcatcaattgaagatccttgcaaaatgtccaaattgtcaagctcctttcaaaatgccagctttatgggaagatggatgctgtcataggtgtagaatgcctttcgcagaaatggcaaaactacagaaggtgtaa

SEQ ID NO: 7 - DNA, 1920 bp, Artificial Sequence
  Synthetic sequence. ShCas12k_pCO1
atgtctcaaatcactatccaagcgcggctcatctcctttgaaagcaacagacaacaattgtggaaacttatggccgacctgaatacgccattgataaacgagttgctatgtcaacttggccagcatcctgactttgagaaatggcaacaaaagggcaaacttcctagcacagtcgtttcccaactctgtcaaccacttaaaaccgatcctcggtttgctgggcaaccttcccgcctctacatgagtgctatccatatcgttgactacatctacaaatcctggctggccatccagaagcgtttgcaacaacaactagatgggaagacgaggtggcttgaaatgctcaattcagacgcagagttagttgaactttccggtgatacactcgaagccattagagttaaagcagcggagattcttgccattgcgatgccagcgtccgaaagcgacagtgcatcccctaaaggcaagaagggcaaaaaggaaaaaaagccatcatcatccagtccaaaacgttcgctgagtaaaacgctattcgacgcctatcaggaaaccgaggacattaagagtaggtcagcgatctcatatctacttaagaatggctgtaaactgaccgacaaggaggaggactctgagaaattcgcaaagaggcgtcgccaagttgaaattcaaattcagcgtttgacggagaaactcataagtcgtatgccaaaaggacgggatctgactaatgcaaaatggcttgaaacactactaactgctaccacgacagtcgccgaagacaatgcacaagctaaacgatggcaagatattctattgacacgctcatcttcacttccgttcccgctcgttttcgaaactaacgaagatatggtatggagcaagaatcagaagggacgtctctgcgttcacttcaacggcttgagcgaccttatcttcgaagtgtactgtggcaatcgacaattacactggttccaaagattcctggaggaccaacaaacaaaacgaaagtccaagaatcagcattcaagcgggctcttcaccttgagaaatggacacctggtatggttggaaggtgagggcaaaggcgaaccctggaaccttcaccaccttactctctactgctgcgttgataaccgcctttggacagaggaggggactgaaatcgtgcgacaggaaaaggcagacgaaatcacgaaattcatcaccaatatgaagaagaagtctgacctctctgacacccaacaggctctcattcaacgtaaacaaagcacccttacacgcatcaacaattcctttgaacgtccttctcaaccactttatcaaggccaaagtcacatacttgtgggtgtttcgctcgggttggaaaagcctgcaactgtggcagttgtggatgctattgcaaacaaagtgttggcctatcggtcgatcaagcagttgctaggggataactacgaactcttgaatagacaaagacgtcaacaacagtacctatcgcacgagcgtcacaaagcgcagaagaacttctccccaaaccagtttggagcttccgagcttggtcaacatattgaccgtctgctggcaaaagccatagtcgcacttgccagaacttacaaggctggttcaatagtgctaccaaaactaggtgatatgagggaggtggtgcaaagcgaaattcaagctatagccgaacagaaattcccagggtatatcgaagggcaacaaaagtatgctaaacagtatcgggttaatgttcaccgttggtcctatggcagactaatccaatctattcaaagtaaagcggcgcagacaggaatcgtcatcgaggaaggaaagcagccgatacgaggctcacctcatgataaggctaaggagctggctctctctgcgtacaacctaagactgacacgacgatcatag

SEQ ID NO: 8 - DNA, 1920 bp, Artificial Sequence
  Synthetic sequence.ShCas12k_pCO2
atgtcacaaattacaatccaagcacgtctcatctcgtttgaaagcaatcgtcaacaactatggaagctcatggccgacttgaacactccgcttatcaacgaactactatgtcaacttggccaacatccggactttgagaagtggcagcaaaagggcaaattaccttccaccgtcgtgagtcaactctgccagcctttgaaaactgatcctcgctttgcgggtcagccttcaaggctttacatgagcgctattcatatcgtggactacatctacaagtcatggctcgcaattcagaaacgtttgcaacagcaattggatggaaagacaaggtggcttgagatgctcaattcagatgctgagttggtagagttaagcggtgatacacttgaggctattcgcgtcaaagccgccgagatcctcgctatcgctatgcctgcatcagagtccgattccgcttctccaaagggcaagaagggcaagaaagaaaaaaagccttctagtagttctcctaaacgatctctctctaaaaccttattcgacgcctaccaggagactgaggatattaagtctcgctctgctatctcctacctgctcaagaatggatgcaaactcaccgacaaagaggaggacagtgagaaattcgctaagcgtcgaagacaagttgaaattcagattcaacgtttgactgagaaactcatatcacggatgcctaaaggtcgtgatcttacaaatgctaagtggcttgagacactcttaaccgcgaccacaactgtggcagaggacaatgcccaagctaagcgctggcaagacatcttactcacacgatcatcaagcctgccattccctctggtattcgaaactaatgaggacatggtatggagtaagaatcagaaggggcggctttgcgtgcacttcaatggacttagtgacttgatattcgaagtctattgtgggaatagacaattgcactggttccaaagattcttagaggatcagcaaacaaaacgtaaaagcaagaatcagcattctagtggactattcactttgagaaatggacatctcgtttggcttgaaggagaaggcaagggagaaccctggaacttgcatcatcttacattgtactgttgcgttgataatcgtctttggactgaggaaggcacagagattgtacgacaggaaaaggctgacgagattaccaaattcatcaccaatatgaagaagaagtctgacctatctgatacacaacaagcacttattcaacgtaaacaaagtacgcttacccggataaacaattcattcgaacgaccttctcaaccactttatcagggtcaatcacacatcctagttggagtttcactcgggcttgaaaagcccgctacagttgcagtggtggatgctattgccaacaaagtgctcgcatatcgctccatcaagcagttattgggcgataactatgaactactaaacaggcaaagacgtcaacagcaatatcttagtcatgaacgtcataaggctcagaagaatttcagtccaaatcagtttggcgcatctgaacttggtcaacatattgaccggttgcttgcgaaggctattgtcgcgcttgcacgaacgtacaaggctggtagcatagtccttccgaaattgggagacatgagggaagtcgttcaaagtgagatacaagcaattgcagagcagaaattccctggctatatcgaaggacaacaaaagtatgcaaaacaatatcgagtgaatgttcataggtggtcttatggtaggctcattcaaagtattcagagcaaggcggctcaaacagggatcgtaattgaggaaggaaaacaacccattagaggatctcctcatgacaaggctaaggagttggctttaagtgcttacaatcttagactgacacgaaggtcataa

SEQ ID NO: 9 - DNA, 1866 bp, Artificial Sequence
  Synthetic sequence. AcTnsB_pCO1
atggcggatgaggagtttgagtttacagagggaaccacacaagtgccggatgctatactgttagacaaaagcaatttcgtggttgatccaagccagatcattctcgctacgtcagaccggcacaaactcaccttcaatctgatacaatggcttgctgaaagccccaaccgcactattaagagtcagcgaaaacaggcggtagcgaacacactagatgtgagcacgaggcaagtcgagagattgctaaaacagtatgatgaggacaaattacgggaaactgctggaattgaacgagcagacaaaggaaagtatcgtgtgtcagagtattggcaaaacttcatcacgactatctacgagaaatcgcttaaggagaaacaccctatttcacccgccagtattgtacgtgaggttaagcgccatgctattgtggatttggaactgaaactcggtgaatatcctcaccaagcaacggtttaccggattctggacccacttattgaacagcagaagcgcaagaccagggtgcggaaccctggctccggctcatggatgacggttgtcactagggacggagaattgctacgcgcggactttagcaatcagatcattcaatgtgaccataccaaattggatgttagaattgtcgataatcacggcaatttgctttcagatagaccctggctaaccacaatcgttgatacctttagtagctgcgttgtcgggttcagattatggattaagcaacccggttctacagaagttgcccttgcattgcggcacgcaattctgcccaagaactatccggaggactatcaacttaacaaaagttgggatgtctgtggtcatccttatcagtactttttcaccgacggtgggaaagactttcggagtaaacatcttaaagcaattggcaaaaagttgggatttcagtgtgaactccgagaccgaccacccgagggtggaattgtggagcggatcttcaaaacgataaacactcaagttcttaaagagcttccaggctataccggagcaaatgtgcaagaacgtccagaaaatgctgagaaggaagcgtgtcttaccatccaagaccttgacaagattctggcgtcattcttttgtgacatatacaatcatgaaccctacccgaaggagcctcgtgatacacgctttgaaagatggttcaagggcatgggaggaaaactaccggagccgctggatgaacgtgagctggacatctgccttatgaaggaggcacaaagagtcgtacaggcacatggctcaattcagttcgaaaacttgatataccggggtgaattccttaaagcacacaaaggtgagtatgtaaccctgcgatatgatccagatcatatactgtcactgtacatctactcgggcgaaactgatgataatgctggtgaattcctcggttacgcccacgcagtcaacatggatacacacgatctgagcattgaggaacttaaagctcttaacaaggaacggagtaatgctcggaaggagcactttaactacgatgccctgcttgcgctcggcaaaaggaaagagttggtggaggagcgtaaagaggacaaaaaagccaagcggaactcggaacagaaaaggctccggtctgcttccaagaaaaacagtaatgttatcgaattgcggaaatcacgcacatctaagtccctcaagaaacaagaaaatcaggaggtgctgccagaacgaatctctcgtgaggagattaagctggagaaaatcgaacaacaaccacaggagaacctgtcggcttcacctaatactcaggaggaggaaagacacaagctcgtgttcagcaaccgccagaagaaccttaacaagatatggtga

SEQ ID NO: 10 - DNA, 852 bp, Artificial Sequence
  Synthetic sequence. AcTnsC_pCO1
atggctcaaccacaattggccacgcagtctatcgttgaagtgctggcccctcgattggatattaaggcacaaattgccaagactattgatattgaggaaatcttcagagcgtgcttcataactaccgatagagcgtctgaatgtttccggtggcttgacgaactcaggatacttaaacaatgtgggaggatcattggcccgagaaatgttggaaagagtcgtgcagcattgcattatcgggatgaagacaagaaacgagtgtcttatgtcaaagcctggagtgcatcctccagtaaacggttgttcagccagatcctgaaagatattaaccacgccgccccaacaggaaaacgccaagacctaaggccccggctggcgggatcactagagctgttcggtttggagctggtcattatcgacaatgcagaaaatctacaaaaggaggcgttacttgaccttaaacaactatttgaggaatgtaatgttccaattgttcttgctggtgggaaggagctagatgatctactgcatgattgcgacttactcacaaacttccccacgttgtacgagtttgaacgtttggaatacgacgatttcaagaaaacgttgactacaattgaattggatgtcctgagtttgccagaagcaagcaatttggcggaaggcaacatcttcgaaatcctggcagtgagtacggaagccaggatgggcattctcatcaagattctgacaaaagcagtgctccattcactcaagaatggattccaccgggttgatgagtcgatcctggagaaaatcgcaagcagatacggcactaagtacataccgctcaagaaccgtaatagagattag

SEQ ID NO: 11 - DNA, 555 bp, Artificial Sequence
  Synthetic sequence. AcTniQ_pCO1
atggcccagaatatcttcctgagtaaaacagaaattggtattgatgaagatgacgaaatacggcctaagctggggtatgtggaaccttatgaggaggaatctatttcccattatcttggacggctacggcgcttcaaagcgaactctctccctagtggatattcacttggcaagatcgctggacttggtgcaatgattagccgctgggaaaagctctacttcaacccattccctacattacaagagctagaggccctatcctcagtcgtcggagttaatgcagataggctaattgaaatgctcccttctcaaggaatgactatgaagcctagacctatcaggctctgcggagcgtgttatgctgaaagtccatgtcacagaatcgaatggcaatgtaaagatcgtatgaagtgtgaccgtcacaaccttcgcttactcatcaagtgcacaaattgtgaaactcctttccctattccagccgactgggtgaaaggtcaatgtccacattgttcattacctttcgctaagatggctaaacgtcagaggcgcgactaa

SEQ ID NO: 12 - DNA, 1929 bp, Artificial Sequence
  Synthetic sequence. AcCas12k_pCO1
atgagtgtcataactattcaatgccgcttagtcgctgaggaagactcactgagacaattgtgggaactgatgtctgagaagaatactccgttcatcaacgagatcctccttcaaattggcaaacatcctgaatttgagacatggttggaaaagggacgcattcccgcagaactgttgaaaacacttggaaactcacttaaaacacaagaaccctttaccggccagcctggtcggttttacacttctgcaataactctcgtggattatctctacaaatcatggttcgcgctacaaaagcggaggaaacagcaaatcgaaggcaaacagaggtggctcaagatgcttaaatcagatcaagaattggaacaagaatcacaaagttcattggaagtcatccgcaacaaagcaacggaactgtttagcaaattcaccccgcaatctgactcagaggcactgcgtcgaaaccagaacgacaaacaaaaaaaagtcaagaaaactaagaaatccaccaaaccgaagacatcctcgatcttcaaaatcttcctatcgacctacgaggaagcggaggagccactgactcggtgcgccctggcctatttgctcaaaaacaactgtcagataagcgaacttgacgagaatccggaggagttcacaaggaacaaacggcgtaaggagatagagattgaacgattgaaagatcaattacaatcacgaataccaaaaggccgtgatcttactggtgaggagtggctggagacactggaaatagcgactttcaatgtgccacaaaacgaaaatgaggcaaaggcgtggcaagcagcgttgctccgtaaaacggctaatgttccctttccggttgcctacgagtcaaatgaggatatgacctggctcaagaatgacaagaatcggctcttcgtgcggttcaacggtttggggaaattgacattcgagatatactgcgacaaacgccatctacattactttcaacgtttccttgaggaccaggaaatacttcgtaattcaaaacgacagcattctagttcgttattcactttgcggagtgggcgtattgcgtggctcccaggggaggaaaagggagaacattggaaagtcaatcagttgaacttctactgttcgctcgatacaaggatgttgacaacagaaggcacacaacaagtagttgaggaaaaggtgaccgcaataacggaaatcctcaacaagactaagcaaaaggatgacttaaatgacaaacaacaggcgttcatcacacggcagcagagcactttggcacggatcaacaatccctttccacgtccgagtaagcccaattaccagggaaagtctagtatccttattggagtgtccttcgggcttgagaaacccgtaactgtggctgtcgttgatgtagtcaagaataaggtcattgcctaccggtccgtaaaacaattgcttggggagaactacaacctactgaatcgacagaggcaacaacaacagcggttgagtcacgagagacacaaggcgcaaaagcaaaatgctccaaactccttcggcgaatcagaacttggacaatatgtggatcggttgttagcggacgcaatcatcgctattgccaagaagtatcaagctgggtctattgtacttccgaagctacgtgatatgcgagaacaaatctcatctgaaattcaatcgagggcggaaaaccagtgtcctggatacaaagagggtcaacaaaagtatgctaaggaatatcggattaacgtgcaccgctggagctacggaagactcattgaatctatcaagagtcaagcagcacaggcgggaatcgccattgaaacaggaaaacaatccatcaggggttctccccaggaaaaggcacgcgaccttgccgtctttacctaccaggaacgtcaagctgcattgatctag

SEQ ID NO: 13 - DNA, 1755 bp, Artificial Sequence
  Synthetic sequence. ShTnsB_pCO3
atgaactcccagcagaaccctgacttggccgtgcacccactggccattccgatggaggggctcttgggcgagagcgccacgacgctggagaagaacgtaattgctacacaactatccgaggaggcgcaagttaagttggaggtgatccagtccctcttagagccatgcgaccggaccacgtacgggcagaagttacgggaagcggccgagaaactcaacgtctccctccgcaccgtccagcggctcgtcaagaattgggagcaagatggcctggtgggccttacgcagacttcccgcgccgacaagggcaaacaccgaatcggagaattctgggagaacttcataacgaaaacgtacaaggagggcaacaagggcagcaagcgcatgacccctaaacaagtcgccctgcgcgtcgaggcgaaagcccgagagttgaaagatagcaaaccgccaaactacaaaaccgtgctgcgagtcctggctcccatcctggagaagcagcaaaaggctaagtctatacgttcgccaggctggcgtggtacaacattgagtgtcaagacccgcgaggggaaagacctaagcgtggactacagtaatcacgtttggcaatgcgaccacacgagggttgacgtactgttggtggatcaacatggagagattctgtctcgcccctggctgactaccgttatcgacacatattcacggtgcattatggggattaacctgggattcgacgctccctcctctggcgttgtggcgctggctttgcggcacgccatactccccaagcgctatggttccgagtacaaactgcactgtgagtggggcacgtacggcaagccagagcacttctacaccgacggtggcaaggactttcgaagcaaccaccttagccaaataggggcgcaattggggttcgtctgccatctcagagaccgtccctccgaaggaggggtcgttgaaagaccattcaaaacgcttaacgaccagttattctcaacccttcccggttacacgggcagtaatgtacaagagcgtccagaggacgctgagaaggatgccagactgactctccgggagctggaacagctcctagtgcgctacatcgttgaccggtacaaccagtctattgatgccagaatgggcgaccagacacgctttgaaaggtgggaggccggacttccgaccgtgcctgttccaattccagaacgcgacctggacatctgccttatgaagcaatcccgtaggaccgtgcagcggggtggctgcttgcagtttcaaaaccttatgtaccggggtgagtacctcgccggttacgccggggaaacagtgaacttaaggtttgacccgcgcgacatcaccacgattctcgtgtaccgccaggaaaacaaccaggaggtgttccttaccagggcgcacgcacaagggctggagacagagcagcttgccctggacgaggccgaggcagcatcgaggaggctaaggacggctgggaaaacaatctcaaatcagtcattgctgcaagaggtggtggaccgggacgctcttgtggctaccaagaagtcccgtaaggagcgacagaaactggagcagactgtgctccggtctgcggcagttgacgagtccaaccgtgagtcgcttccgtctcaaattgtggagccagatgaagtggaaagcaccgagaccgtgcattctcagtatgaggacattgaggtctgggattacgaacagctccgggaggaatatgggttctga

SEQ ID NO: 14 - DNA, 1755 bp, Artificial Sequence
  Synthetic sequence. ShTnsB_pCO4
atgaacagccagcagaatccggatctcgccgtgcatccgttggccataccgatggagggattgctcggcgagtccgctactacgcttgagaagaatgtcatcgcaacacagctcagtgaggaggctcaagttaaactggaggtgatacagtctctcctggaaccgtgcgaccgcacaacttatggccagaagctccgggaggccgccgagaaactcaacgtctcacttagaactgtgcaacggctcgttaagaactgggaacaggacggcctggtcggccttacacagacgagccgcgcagacaaaggcaaacacaggatcggggagttttgggagaactttatcaccaagacttacaaggagggcaacaagggaagcaaacggatgactccgaagcaagtggccttgcgagtcgaggcaaaggccagggaattgaaagactcaaagccgccgaattacaagacggtccttcgcgtgctcgccccgatcttggagaaacaacagaaggctaagtcgattcggtcaccaggatggaggggaactaccctgagcgttaaaacgcgcgaggggaaagaccttagtgtggattactcaaatcacgtttggcagtgcgatcatacccgagtggatgtgctactcgtcgatcagcacggggagatactttctaggccgtggctgacaacggtgattgacacttattcacgctgcattatggggattaacctggggttcgacgcaccttcaagcggcgtggttgcacttgcgcttcgccacgccatcctgcctaaacgctatggctccgagtacaaactgcattgcgaatggggcacatacgggaagcctgagcacttctacacagatggcggcaaagacttccggagcaaccatctaagccaaataggggcccagttggggttcgtttgtcaccttagagatcgcccatccgaaggcggcgtcgtggagagaccattcaagactctgaacgatcaattgttctcaacactcccaggatataccggctcaaatgtacaggaacgtccggaagatgcggaaaaggacgctcgcctcaccctgcgtgagcttgaacagttgctcgtgcgctacatcgtggaccgctacaatcagtcgattgacgctcgcatgggagaccaaaccagattcgagagatgggaagcgggcctcccgactgtgccagttcctatccccgagcgagaccttgacatctgcctcatgaagcaatcacgtagaaccgtccagaggggtgggtgcctgcaattccagaacctcatgtaccggggagaatacctcgctggttacgccggagagacagtgaatctgcgctttgacccacgggacataaccacgatcctcgtatatcgccaggaaaacaatcaggaagtgttcctgacccgcgcgcacgcgcaaggactggagaccgagcaacttgcacttgacgaggccgaagcagcctcacgaagactgagaaccgccggaaagaccatctctaatcagtcgctcctccaagaggtcgtggatagagatgccctggtcgccaccaagaaatcgcgtaaggaacgacagaagctcgaacaaaccgtcctccgctcagccgcagtagatgagtcgaaccgcgagtctttgccgtctcagattgtcgaaccagatgaggtggagagtaccgaaacagttcactcgcagtatgaggacattgaggtctgggattatgagcagttgcgggaggagtatggcttctaa

SEQ ID NO: 15 - DNA, 1755 bp, Artificial Sequence
  Synthetic sequence. ShTnsB_pCO5
atgaactcacaacaaaatcctgatcttgctgttcatccattggctattcctatggaaggtctattgggcgaatctgcaacaactcttgagaagaacgttattgctactcaactttcagaggaagctcaagtaaaacttgaagtcatccaatcattgctagaaccttgtgaccggacaacttacggacaaaagctccgtgaagctgccgagaaactaaatgtttcacttcgtactgttcagaggcttgtcaagaactgggaacaagatgggctcgttgggcttacacaaacctctcgtgcagataagggaaaacaccgaataggggagttttgggaaaacttcataactaaaacttacaaagagggcaataagggttccaagcgtatgactccaaaacaagttgccttaagagttgaagcaaaggctcgggagcttaaggactctaaaccacctaactacaaaactgtccttcgcgtattggcacccatcctggagaagcagcagaaggctaagtcaattcggtcgcctggatggcggggtactactctgagcgttaagacacgagaaggcaaagacttgtctgtggactacagtaatcacgtttggcaatgcgaccatacgagagttgatgtcttactggtggatcaacacggagaaattctgtcccgaccctggcttactaccgttattgatacttattcaaggtgcataatggggatcaacctcggctttgacgcaccttcaagcggcgtggttgcattagcgcttcgacacgctatactccctaaacggtatggttcagagtacaaattgcattgcgaatggggcacatacggcaaaccagaacacttttacacggatggtgggaaagacttccggtctaatcatctatcacagataggtgcacagcttgggttcgtctgtcatctgagagataggccaagtgaaggaggggtcgtggaacgcccattcaaaacattgaatgaccaactctttagcacgcttccgggatacaccggatctaatgtacaagaacgaccagaggatgcggaaaaggacgcccgtttgacactgagggagcttgaacaacttttagtgcggtatatcgttgatcgctacaaccagtctattgatgcaagaatgggagaccaaacgcgatttgagagatgggaggctggattgcctacggttcctgttcctatccctgagcgcgatctggacatctgcctgatgaagcaatctcgccgtacagtccaacgaggaggttgcttgcaatttcaaaatctaatgtaccgtggagagtatctcgctggatatgctggagaaactgtcaacttaaggtttgaccctagagatattacgactatcttagtgtaccgtcaggaaaacaatcaggaggtcttcctcacaagagcacatgcacaaggacttgagactgagcaactagccttagatgaagcagaagcagcatccaggcgattacgaaccgctgggaaaactatttccaatcagtcgttgctacaggaagtggtggacagagatgcactggttgcgaccaagaagtcccgtaaggagcgccaaaagttagaacaaacagttctgagatccgcagccgttgacgaaagtaatagggagtccctcccttctcaaatcgtggaacctgatgaagtggaatctacagaaaccgtgcattctcagtatgaagatattgaagtctgggattatgaacagttgcgggaggaatacgggttctga

SEQ ID NO: 16 - DNA, 828 bp, Artificial Sequence
  Synthetic sequence. ShTnsC_pCO3
atgaccgaggcccaggccatcgccaagcagctcggcggagtgaaaccggatgacgaatggctccaggccgagattgcgcgcttgaagggcaagtctatcgtgccactgcaacaagtgaagacactgcacgactggcttgacggaaaacgcaaagctcggaagtcctgccgggtggttggagagagccgtacgggcaagactgtcgcgtgtgacgcctatcgctaccgccacaagccccaacaagaggctggaagaccgccaacggtcccggtcgtttacatccgtccgcaccagaagtgtggccccaaagacctattcaagaagatcaccgagtatctcaagtaccgcgtgacgaaggggacggtcagtgactttcgggacaggacaattgaagtgctgaaaggctgcggagtagagatgctgataattgacgaagctgatcgactcaagcctgagaccttcgcagatgttagagacatcgccgaagatttggggattgcggttgtcctcgttggcacagacagattggatgcagtgattaagcgtgacgaacaagtgcttgagcggtttcgcgcgcatctgcggttcggcaaactgtcgggcgaggactttaagaacaccgttgaaatgtgggagcaaatggtgcttaagctccctgtgagcagcaacttgaagtctaaggaaatgctcagaatcttgaccagtgccacagaggggtacatcgggaggctggacgagatcctacgggaagcggccatcagatcgctaagtaggggcctcaaaaaaatcgacaaagccgtgctgcaagaggttgctaaggagtacaag

SEQ ID NO: 17 - DNA, 828 bp, Artificial Sequence
  Synthetic sequence. ShTnsC_pCO4
atgacagaagcccaagctattgccaagcagctcggcggggtcaagcccgatgacgagtggctccaagcagagatcgcccgcctcaaaggcaagagcatcgtccctctccaacaagtgaagaccttgcacgactggctcgacggcaagcgtaaggcccgcaaatcctgccgcgtggtgggcgagtcccggacaggcaagacggtcgcttgtgatgcctaccgataccgccacaagccccaacaggaggcgggtcggccgccgactgttcccgtcgtgtacatccgcccacaccaaaagtgcggcccgaaagacctcttcaaaaagataacggagtacctcaagtaccgggtgaccaaaggcactgtctccgactttcgggaccggacaattgaagtgctcaaaggctgcggagtggaaatgttgataattgacgaggcagatagacttaagccggagacgttcgccgacgtgagagacatcgccgaggatctaggaattgctgtagtcctggtcgggacagaccgcctggatgctgtcattaagcgggacgagcaagtgttggagcggttccgcgcccacctgaggttcggcaaattgtctggcgaagacttcaagaacacggtggaaatgtgggagcagatggtccttaagctgcccgtgtcgtcgaatcttaagagcaaggagatgttgaggatactcaccagcgcgaccgaagggtacatcgggcgactggacgagattctgcgggaggcggcgatacgctctctcagtcggggcctcaagaaaatcgacaaagctgttctgcaagaggtggcgaaggagtacaag

SEQ ID NO: 18 - DNA, 828 bp, Artificial Sequence
  Synthetic sequence. ShTnsC_pCO5
atgacagaggcccaggccattgccaagcagttgggaggtgtaaaaccagacgacgagtggctccaagccgagatagcaagacttaaggggaagtcaatcgttcctctgcaacaagtgaaaacacttcatgattggctggatggaaaacgtaaagcaaggaaaagttgccgtgtagtaggtgaatccagaacaggcaaaacggtggcttgtgatgcctaccggtacagacacaagcctcaacaagaggccgggagaccacctaccgtcccagtcgtgtacattagacctcatcagaaatgtggtcctaaagacttattcaaaaaaatcactgagtatttgaagtaccgtgtaactaaaggcacagtgagcgactttcgtgaccgtactatcgaagtgttgaaaggatgtggtgttgaaatgctcattatcgacgaagcagacagactaaagccagagacattcgcggatgttcgtgatatagcagaggatctcggcatagcagtagtcttagtcggtactgacaggcttgatgctgttatcaagcgggacgaacaagtattggagcgttttagagcacaccttcgatttgggaagctgtctggggaagactttaagaacacagttgaaatgtgggaacaaatggtgcttaaacttccagtgtcatctaatcttaaaagcaaggaaatgttacgcatacttacatctgctaccgagggttacatcggacgcttagacgaaatactcagggaggcagccatccggtctttatcacggggcctcaagaagatagacaaggctgtgttacaggaggttgctaaggagtacaaa

SEQ ID NO: 19 - DNA, 504 bp, Artificial Sequence
  Synthetic sequence. ShTniQ_pCO3
atgatcgaagcgccggacgtgaaaccgtggcttttcctcatcaagccttacgaaggcgagagcctcagccactttctcggccgcttccgccgcgcgaaccatctgtcggcatccggcctcgggactttggccgggataggggctattgtcgctcggtgggagcgcttccactttaacccgagaccttcccaacaggaactggaagctatagcctccgtggtggaggtcgccgctcaaaggctcgcccagatgctgcctccggctggagtggggatgcaacatgaaccgatccgcctctgtggggcatgttacgccgaatctccctgtcaccgcatcgaatggcagtacaagtctgtctggaagtgcgaccgccaccagttgaagatacttgctaagtgcccaaattgccaggctccctttaagatgcctgctctttgggaggatggttgctgccatcggtgtagaatgccgttcgctgagatggcgaaactacaaaaggtgtaa

SEQ ID NO: 20 - DNA, 504 bp, Artificial Sequence
  Synthetic sequence. ShTniQ_pCO4
atgattgaagctcccgatgtgaagccgtggcttttcctcattaagccatacgaaggtgagtctctctcacacttcctgggtcgtttcagaagggctaaccacctcagcgcctccggcctgggcacgctcgcggggatcggagccatcgtggctcgctgggaacgcttccacttcaatccgcgaccgtcccaacaggaattggaagcaattgcgagtgtggtcgaagtggccgcgcagcgactggctcaaatgttgccgccagccggtgttgggatgcagcacgagccgatccggctgtgtggtgcttgttacgctgaaagcccgtgtcacagaattgagtggcagtacaagagtgtgtggaaatgcgacaggcaccagcttaagattcttgcgaagtgcccgaactgtcaagcgccgttcaaaatgcccgccttatgggaggatggttgctgtcaccggtgtcgtatgccattcgcggagatggctaaactccagaaggtctga

SEQ ID NO: 21 - DNA, 504 bp, Artificial Sequence
  Synthetic sequence. ShTniQ_pCO5
atgatcgaagcacctgatgttaaaccgtggctgttccttattaagccttatgaaggcgaatcactatcacactttctcggacgcttccgacgcgctaaccacttgtcagcatcaggactgggaacattggccggtattggggccattgtggcaagatgggagagattccacttcaatcctagaccatctcaacaagaactggaggctatcgcctcagtagttgaagttgcagcacaaagattggcccagatgcttcctccggctggagttggtatgcagcatgagcctatcagactttgcggcgcttgttatgcggagtctccatgtcacagaattgaatggcagtacaaatctgtgtggaaatgcgacagacatcaactcaagattcttgcaaagtgtcctaattgtcaagcacctttcaaaatgccagcactttgggaagatggatgctgccaccgttgcagaatgccctttgctgaaatggctaaactacaaaaggtctga

SEQ ID NO: 22 - DNA, 1920 bp, Artificial Sequence
  Synthetic sequence. ShCas12k_pCO3
atgtcacagatcaccatccaagcacgtctcatctcgttcgagtcaaatcgccagcaactctggaaactcatggccgatctgaacacgccgctcattaacgagcttctgtgccaactcggccaacaccccgacttcgagaagtggcaacagaaggggaagctcccctccactgtggtgtcgcaactatgccagcccctcaagaccgatcctcgattcgcggggcagccctctcgtctctatatgagtgcgattcatattgtggactacatctacaaatcatggctggcaatccagaaacgcttgcagcagcagctcgacgggaagacccggtggctcgaaatgctaaactcagacgcagagctggtggagctgtcgggtgacacactagaggccatccgcgtcaaggccgccgaaatcctagccattgcgatgccagcatccgagtcagactccgcgagcccgaagggcaaaaaaggcaagaaagagaagaagccctcatcatcctcgcccaagcggtcactgtccaagacgttattcgatgcctaccaggaaacggaggacatcaagtccagatccgcaatatcctacctcctcaagaacggctgcaaactcactgataaggaggaagactcggagaaattcgcaaaacgtcgaagacaagtcgagattcagatccaaaggctcactgagaaactcatctcacgtatgccgaaaggtcgggatctgactaatgctaagtggctggagaccctcctaaccgctacaaccacggtggccgaggacaacgctcaagcaaaacgctggcaagacatacttctcactaggagcagcagtttgcctttccctctcgtattcgagactaatgaggatatggtgtggagcaagaatcagaaggggagactctgtgtgcacttcaacgggctgtctgacctaatcttcgaggtgtactgtggcaaccgccagctccactggttccagcgattcctggaagatcagcagaccaaacggaagtccaagaaccagcatagtagcggtctattcacactgaggaacggtcacctggtgtggcttgagggtgaaggcaagggcgagccctggaaccttcaccatctgacgttgtactgttgcgtcgataacaggctgtggaccgaggagggaactgagatcgttcgtcaggaaaaggctgacgagattaccaagttcattacaaacatgaagaagaaatcggacctgtcagatacacagcaagcgctcatccaacgtaagcagtcaaccctgaccaggatcaataacagtttcgagcgccccagccaacccctttaccaggggcagagccatatcctcgtgggtgtctccctggggctggagaaacccgccaccgtggcggtggtggacgccattgcaaacaaagtcctggcctatcggagtattaagcagctcctcggagataattacgagctgctaaaccgccagaggaggcagcagcagtacctctcccacgaacgacacaaggcccagaagaatttcagccctaaccagtttggcgcttccgaactcggacagcacatcgaccgactgctcgcaaaggccattgtcgcactcgcccggacgtacaaggccgggagtattgtgctgcccaaactcggcgatatgagggaagtcgttcagtccgagatacaagctatcgcagaacagaaattccccggctacatcgaggggcagcagaagtatgcgaagcaataccgtgtaaatgtccaccgctggtcgtacggacgtctaattcagtcaatccagtcaaaagctgcccagaccggcatcgtgatcgaggaggggaagcagcccatccgaggctcgccccacgataaggcgaaggagctggctctatcggcctacaatctccgactcacacgccggtcctag

SEQ ID NO: 23 - DNA, 1920 bp, Artificial Sequence
  Synthetic sequence. ShCas12k_pCO4
atgtcccaaattaccatccaagcgagactcatctccttcgagagcaaccggcagcagctctggaagctcatggctgatctgaatacgcccctcatcaacgaactactctgccaacttgggcagcacccggactttgagaagtggcagcagaagggcaaactcccctcgacagtggtcagccaactctgccagccgctcaaaacggacccacgcttcgcgggacagccgtcccgcctctatatgagcgcgattcatatcgtggactacatctacaagagctggctcgccatccaaaagcggcttcagcagcaactggacggcaagactcgctggctcgaaatgttgaacagtgacgcagaactggtcgagttatccggtgacacgctggaggccatccgcgtcaaggcggctgagatcctggcgatagcgatgcctgcgagcgaaagtgactccgccagcccgaagggcaaaaagggcaaaaaagagaagaagcccagctccagctcccctaagcggagccttagtaagaccttattcgacgcctaccaggaaaccgaggacatcaagagtcgcagcgccatatcgtacctcctcaagaatggctgcaaactgactgacaaagaggaggacagtgagaagttcgctaagcgacgacgccaagttgagatacagattcagcgcctgacggagaagctcatctctcggatgcctaagggacgcgacctcacaaacgcgaagtggcttgagactctcctcacagccacaacaacggtggccgaggacaacgcccaggctaaaaggtggcaagacatcttgctaactcgtagctcatcactcccgttcccactcgtctttgagacaaatgaggacatggtgtggagcaagaaccagaagggacggctctgcgtccacttcaacggcttaagcgaccttatcttcgaggtgtactgcgggaatcggcaattgcactggttccagcgctttctggaggatcagcagactaagaggaagtccaagaaccagcactctagcggcctgttcacccttcgcaacggtcacttggtctggctggaaggtgaaggaaagggcgagccctggaacctgcatcacttaaccttgtactgctgcgtggacaaccgcctttggacagaggaaggcaccgagatcgtgcgacaggagaaggcagacgagattaccaaattcataacgaacatgaagaagaaatccgacctgtcagacacgcaacaggcactgatacagcgcaaacaaagcacattgactcgcattaacaactcctttgagcgtccgtcgcagcctctttatcagggccagtcgcacatcctggtcggggtgtcgctcggactggagaagccagccacggtggccgtggtggacgccatcgccaacaaagttctagcatatcgcagtattaagcaattactcggcgacaattacgaactactgaatcggcagagacgtcaacagcagtatctctcccacgaacgccacaaggctcaaaaaaacttcagtccaaaccagttcggagcgagcgagctgggtcagcacatcgacagacttctcgctaaggccatcgtggcacttgcccgaacctacaaagcgggctcaatcgtgctccccaagctcggcgacatgagggaggtggtgcaaagtgagatccaggccatcgctgagcagaaattccctggctatatcgagggccagcagaaatacgcgaagcagtatcgcgtgaatgtgcatcggtggagctacggccgtcttatccagtcgatacagagcaaagccgctcagactggaatcgtcattgaggagggaaagcaacccatccggggaagcccgcacgataaggctaaagagttggccctgtctgcctacaacctccgcctgacacgccggtcctag

SEQ ID NO: 24 - DNA, 1920 bp, Artificial Sequence
  Synthetic sequence. ShCas12k_pCO5
atgagccagattaccatccaagctcgcctaatcagcttcgagtcgaatcgacaacaattgtggaagttgatggctgatctcaatacccctctgattaacgagctgctctgtcaattgggacagcacccggactttgaaaagtggcagcaaaagggcaagctcccttcaacggtagtttcccagttgtgccagccactcaaaactgacccaagattcgctggccaaccctctaggctctatatgtctgcgattcatatagtggactacatatacaaatcttggcttgcaatccagaagcgattacagcaacagcttgatggcaagacaagatggttggagatgttaaacagcgatgctgagctggtcgaacttagcggcgacactctggaggctattagagtcaaagcagccgaaatactcgctatcgcaatgcccgcatctgagtcggactctgcttctcctaaaggcaaaaaggggaaaaaagagaaaaagcccagtagctcatcacccaagaggagtctttcaaagacactcttcgacgcttatcaggaaaccgaagacattaagtctaggagtgctatcagctatctgctcaagaatgggtgcaaactcactgacaaggaggaggacagcgagaaattcgctaaaaggcggcgtcaagttgagatccagattcagagactcactgagaagctcatctcaagaatgcccaaaggccgagatttgacaaatgctaaatggttggagaccctccttactgctactactactgtcgcggaagataacgcacaggctaagcggtggcaagacatcttacttactcgttcatcatccttacctttcccacttgtcttcgaaacaaacgaggatatggtgtggtccaagaatcagaagggacgtctctgcgtgcactttaacggactttcagaccttatcttcgaggtctattgtggaaatcgacagttacactggttccaacgtttcctggaagatcagcaaactaaacggaagtccaagaatcagcattctagcggccttttcaccttgcggaatggccatctagtatggcttgagggcgaaggaaagggtgaaccctggaaccttcatcatctgacactatactgctgtgttgataatcgtctatggacagaggagggaacagaaatagtaagacaggaaaaggctgacgagatcaccaaattcatcacgaatatgaagaaaaagtccgacctatctgatactcaacaggctctgatacagaggaagcagtccactttaactcggatcaacaattcctttgagcgcccatcccaaccactctatcaaggccaatcacacatcctcgtgggcgtgtcactgggcctggaaaagcctgctacagttgcggtagtggacgctattgcaaacaaagtcttggcctaccgatctatcaagcagcttctgggagataattacgaactgttgaaccgacagagacgtcaacagcaatacctcagccatgagagacacaaggcacagaagaatttcagtccaaaccagttcggagcaagcgagttggggcagcatattgacagactcctcgccaaagccattgtggccttagccagaacatacaaggccggttcaattgtactccctaagctcggtgacatgcgtgaggttgtgcagtcggagatacaagcaattgcagaacagaaattccccggctacatcgagggccagcaaaagtatgccaaacaataccgggtcaatgtacatcgttggagttacggccgacttatccagagcattcagtctaaagccgcacagactggcattgtcattgaggaagggaaacagcccatcagaggaagtccgcatgataaggctaaagagttggccttgagcgcgtacaacctgagattaactaggcgttcctaa

SEQ ID NO: 25 - DNA, 1866 bp, Artificial Sequence
  Synthetic sequence. AcTnsB_pCO2
atggccgatgaggaatttgagtttaccgagggcacgacccaagtccctgacgccatcctcctcgacaagagcaacttcgtcgtggacccgagccaaatcatcctcgccacgagcgaccgccacaagctcaccttcaacctcattcaatggcttgcggaaagcccgaaccgcacgatcaagtcccaacgcaaacaagccgtggccaacactctggatgttagcacgcgacaagtcgagcgcttactcaaacagtacgacgaggacaagctgcgggagacggccgggatcgagcgcgcggacaagggcaagtaccgggtatccgaatactggcaaaacttcatcacgaccatctacgagaagtcattgaaggaaaagcaccctatctcaccggcgtccatcgtccgagaagtcaagcgccatgccatcgtggatctggaactcaaactgggcgagtacccacaccaggcgacagtttatcgcattcttgacccgctcatcgagcagcaaaagcgcaagactcgcgtccggaaccctggcagtggttcgtggatgaccgtggtgacccgcgacggcgagctgctccgggctgactttagcaaccagatcattcagtgtgaccacaccaagttggacgtccggatcgttgacaatcacgggaatctcctctccgaccgcccgtggctgaccactatcgtggatactttctcatcatgtgtggtgggcttccgtctctggatcaagcagcctggcagtacagaggttgccctcgcgttgcggcacgccatcttacctaagaattaccccgaggactaccagctaaacaaaagctgggatgtctgcgggcatccgtaccagtacttcttcacagacggaggcaaggacttccggagcaagcacctcaaggccatcggcaagaaactggggtttcagtgcgagctgcgggacaggccgcccgagggagggatcgtcgaaaggatattcaaaaccatcaacactcaagtgctaaaggagctacccggatacaccggcgcgaacgtgcaagagcgcccggaaaatgctgagaaggaggcgtgcctgactatacaagacttggacaagatcctggcgtcattcttctgtgacatctacaatcacgagccgtacccaaaggagcccagggacacgcgcttcgaaagatggttcaagggcatgggcggcaaactccctgaaccccttgacgaaagggaacttgacatctgcctcatgaaggaggctcagcgggtagtccaggcacacgggagcatccagttcgagaacctgatctatagaggagagttcttgaaagctcacaagggtgagtacgtgaccctgcgttacgaccccgatcatatcctgtcactgtacatctactcgggcgagacggatgataacgccggagagttcctgggttacgcccacgccgtgaacatggatacccacgatctgagcatcgaggaactcaaagcgctcaacaaggagcggtcgaacgcccggaaggagcacttcaactacgacgcgctcttggccctgggcaagcggaaggagctggtagaggaacgcaaggaggacaaaaaggccaagcgcaactcagagcagaaacgcctccggagcgcctccaaaaaaaacagtaacgtaattgaactgaggaaaagccgtaccagtaagtccctcaagaaacaagaaaatcaggaggtgttacccgaacgtatttcccgcgaggagattaagttggagaaaatcgagcagcagcctcaggaaaacctgtcagccagcccgaatacccaggaggaggagagacacaagctggtcttcagcaaccgccaaaaaaatctaaacaagatatggtag

SEQ ID NO: 26 - DNA, 1866 bp, Artificial Sequence
  Synthetic sequence. AcTnsB_pCO3
atggcagacgaggagttcgagtttacagaggggacgacccaagtaccggacgctatactcttggacaagagcaatttcgtcgttgacccgtctcagattatcctcgccacctcggacaggcacaagctcacgttcaacctgatccagtggcttgcagagtctcccaaccgcacgattaagagccagcggaagcaagccgtggcgaacacgctagatgtttcaacgcgacaagtagagcggctgctcaagcagtacgatgaggacaagctccgtgagaccgccgggattgagcgggccgataagggcaagtaccgtgtgagtgagtattggcagaacttcatcaccacgatatacgagaaatcccttaaggagaagcaccccatctccccggcgtccatagtgcgggaggtgaagcgtcacgctatcgtggacctggagctaaaactaggggagtatccgcaccaggccaccgtgtaccgtatccttgacccgctgatcgagcagcagaagcgcaagactcgggtgcgtaaccccggaagcggctcctggatgaccgtagttacacgcgacggcgaactgctgagggcagacttttcaaaccagattatccagtgcgatcacacgaagttggacgtgagaatagtggacaatcacggcaaccttctgtctgaccggccgtggcttacaacgattgtggacacattcagtagttgcgtcgtgggattccgcctgtggatcaagcaacctggttcgaccgaggtggccctcgcccttcgccatgcaatcctgcccaagaactatcccgaagactatcaattgaacaaaagctgggacgtctgcggccacccgtatcagtacttctttaccgatggcggcaaagacttcagaagtaaacatctcaaagcaatcggcaagaagctcggtttccaatgtgaactccgcgaccgcccacccgagggcgggatcgtggagcgcatattcaagaccatcaacactcaagtcttgaaggaacttccgggctacactggtgcgaacgtacaggagcgcccagaaaatgccgagaaggaggcttgtctcaccatccaagaccttgacaagatacttgcgagctttttttgcgacatctacaaccacgagccttacccaaaggagccccgtgacaccagattcgaacgatggttcaagggtatgggtggcaagctcccggagcccctagacgaacgggaattggacatctgccttatgaaggaggcccagcgagtcgtccaagcacacggcagcatccagttcgagaacttaatctatcggggcgagttcctcaaagcacacaagggcgagtacgtgaccctgcggtacgacccggatcacattctctcactctacatatacagcggagagactgacgacaacgccggagaattcctcggttatgctcatgcagtgaacatggatacgcacgacctctccattgaggagttgaaggccctcaacaaagagcggtcgaacgcccggaaggagcacttcaactacgatgccctgctggcgcttggaaagcggaaggagctggtggaggaacgaaaggaggacaaaaaggcaaagcgtaacagtgaacagaaacggttgcgctctgcgagcaaaaagaatagcaacgtcatcgagcttcggaaaagccgaaccagcaagtcgctcaaaaagcaggaaaaccaggaggtgttgcccgagcggatctcccgtgaggagatcaagctggagaagatcgagcagcagccccaggagaacctgtcggcctctccaaacacccaggaggaggaacgccataagctcgtcttcagcaaccgtcagaagaacctcaacaagatatggtga

SEQ ID NO: 27 - DNA, 1866 bp, Artificial Sequence
  Synthetic sequence. AcTnsB_pCO4
atggctgacgaggagtttgagttcaccgagggtactactcaagtacccgatgccatcctactagacaagagcaatttcgtggttgaccctagtcagatcattctcgccacttctgatcgccacaaactgactttcaacctcattcagtggctcgctgagtcaccgaacaggacgatcaagtctcagcgaaagcaagcagtggcaaacacactcgatgtgagtaccagacaagtcgaacgcctgttgaaacagtatgatgaggataagttacgagaaaccgctggtatcgaacgtgccgacaagggcaaataccgtgtgtcagaatactggcaaaacttcataacaactatctacgagaagtctctaaaggaaaagcaccctatctcacccgcctcaatcgtgcgcgaagttaagcgccatgctatcgttgatcttgaattgaagctgggcgaatatccgcatcaagctacagtttaccgcatcttagacccgcttattgagcagcagaagcgtaaaaccagagtacggaatccaggatcagggtcatggatgaccgtcgtaacacgcgatggggaactcttgcgcgccgacttttcaaaccagatcattcaatgcgatcatacaaagttggatgtgagaatcgtggataatcatggaaatctgctttctgatcgtccctggctgacgaccatagtggacaccttctcgtcatgcgttgtggggtttagactttggatcaagcaaccaggcagtacagaggttgctctcgcattgcgccatgccatcttgcccaagaactaccccgaagactaccagttgaacaagtcatgggacgtctgcgggcacccataccaatatttcttcacagatggagggaaggacttccgctctaagcaccttaaggccattggcaagaaattgggttttcagtgcgagttgagggacagaccacctgagggtggaattgtggagcggatcttcaaaactatcaacacccaagtgcttaaggagcttcctggctacaccggcgcaaacgtacaggaaaggccggaaaacgctgaaaaggaggcttgtcttaccatacaagatttggacaagattcttgcgtcgttcttttgcgacatatacaatcatgaaccttatccgaaggagccgcgagatacaaggttcgaaagatggttcaagggtatgggtggcaaattgcctgaaccgctcgatgagcgcgagcttgacatctgccttatgaaggaggcacagagagtcgtgcaagctcacgggagcatccagtttgagaacctgatatacaggggagagttcctaaaggctcacaaaggcgaatacgttacattgagatatgatcctgaccacatcctgtcattgtacatctatagtggggaaaccgatgataatgccggtgagtttctcgggtacgctcatgctgtgaatatggatactcatgaccttagcatcgaggagttgaaggctctgaacaaggaacgctcaaatgcgaggaaggagcactttaactacgatgcacttctcgctctgggcaagcgcaaggagttggtggaggaaaggaaagaggacaaaaaagctaaacgtaatagtgaacaaaagcgattgcgatcagcttccaagaagaatagtaatgtgattgagctacgaaagagtagaacttccaagagtctcaaaaagcaggaaaatcaggaagtgctacctgaacgtatatcaagggaggagattaagctggagaagatagagcaacagcctcaggagaacttatccgccagtccgaacacacaggaggaggaacggcacaagttggtgtttagtaatagacagaagaatctaaacaagatatggtag

SEQ ID NO: 28 - DNA, 852 bp, Artificial Sequence
  Synthetic sequence. AcTnsC_pCO2
atggcgcaaccgcaactggccactcagagcatcgtcgaagtgctcgcaccgcgccttgacatcaaggcccagattgcgaaaaccatcgacattgaggagatattcagagcctgcttcatcacaaccgaccgggcgagcgagtgcttccggtggctcgacgaactccgcatccttaaacaatgcggcagaatcatcggccccagaaacgtcgggaagtcgcgtgctgcgctgcactatcgagacgaggacaagaagcgggtatcttatgttaaggcgtggtccgcgtcgtcctcaaaacgccttttcagccaaattctgaaagatattaaccatgccgccccaacgggcaaacgccaggacttacggccccggctcgctggctcgcttgaactattcgggctggagcttgtcattatcgacaacgccgagaaccttcagaaggaggcccttctcgaccttaagcaactcttcgaggagtgcaacgtgcccatcgtcctcgcgggcggcaaggaactggacgatctgctccatgactgcgacctcttgacaaactttcctaccctctacgagttcgagcggctggagtacgacgacttcaagaaaacgctgacgacaatagagctggacgtcctcagcctaccagaggcttccaacctggccgagggcaacatattcgagatcctggctgtgtcaaccgaggcgcggatggggatactcatcaagatcctgacaaaggccgtgctccatagccttaagaacgggtttcacagggttgacgagtcgatcctggaaaaaatcgccagccgatatggcacgaagtacattccgcttaagaatcggaaccgggactag

SEQ ID NO: 29 - DNA, 852 bp, Artificial Sequence
  Synthetic sequence. AcTnsC_pCO3
atggcgcaaccacaactcgccacccagtccatagtggaggtgctcgcgccccgcctggacatcaaggcacagattgccaagactatcgacattgaggaaatcttccgggcctgcttcattaccaccgacagagcttcggagtgcttccggtggctcgacgaacttcggatactcaaacagtgtgggcgtatcattgggccacggaacgtcgggaagtcccgcgcggcgctgcactaccgcgatgaagacaaaaagcgggtgtcgtatgtgaaggcttggtccgcgagtagctccaagcggctgttcagtcaaattcttaaggacatcaaccatgctgcgccgacgggcaaacggcaagacctcaggccgaggctggctggatcgctggagctgttcggacttgagctggtcataattgataatgcagaaaacctccagaaggaagcgctgctcgaccttaagcagttgttcgaggagtgcaatgtccctattgttctcgctggtgggaaggagctggatgatctgctgcacgactgcgacctgcttaccaacttcccaacgctgtacgagttcgagcggctggaatacgacgacttcaaaaagactctcaccaccattgagctggatgtcttgtctctgccggaagcgagtaatctcgcggaggggaacatcttcgagatcctagcagtcagtacggaggcacgcatgggcatcctcattaagatcctcactaaagctgtgcttcattccctcaagaacggcttccaccgggtagatgagtccatccttgagaagattgcctcacggtatggtacgaagtatatcccgcttaagaaccgtaaccgcgactga

SEQ ID NO: 30 - DNA, 852 bp, Artificial Sequence
  Synthetic sequence. AcTnsC_pCO4
atggcacaacctcagttggcaacccagtctatcgtggaggtgttagcaccgaggttggacattaaggctcagatagcgaaaactattgacatcgaggaaatcttcagagcttgctttatcacaaccgacagggcctccgaatgcttcagatggctcgacgaattacggatcttgaaacagtgcggtcggatcatcggcccacgcaacgtcggaaagtccagagctgccttacattaccgcgatgaggacaagaaacgggtttcctatgtcaaagcctggtcagcctcttcctcaaagcgactattctcacaaattcttaaggacatcaatcacgctgcacccactgggaaaaggcaagacttgagaccaagacttgctggtagtctggaattgttcggtttggaactcgtcatcatcgacaatgctgaaaatctacaaaaggaagcactacttgacctgaaacagctattcgaggagtgtaatgtaccaatcgtcctggctggcggcaaggaattggatgacttattgcacgattgtgacctgcttacgaactttccaaccctctacgagttcgagcgccttgagtacgacgatttcaaaaagacactgacaacaattgagttggacgtgctgtctctgccagaggccagtaacttggcagaggggaatatcttcgaaattctagcagtctcaacagaagcccgtatggggatactcatcaagattcttactaaggccgtgttgcattcactcaagaatgggttccaccgagttgacgagagtatccttgagaagattgcatctcgctatggtacaaagtacattcctctcaagaatcgtaaccgtgattaa

SEQ ID NO: 31 - DNA, 555 bp, Artificial Sequence
  Synthetic sequence. AcTniQ_pCO2
atggctcagaacatattcctctcgaagacggagatcggaatcgacgaggatgacgaaatccgaccgaagctcgggtatgtggagccgtacgaggaggagtcgatcagccattacctcggccgcctccgaagattcaaggccaactccctgccgtcggggtattcgctcggcaagatcgctggcctgggtgctatgatctcccgttgggagaagctctatttcaaccccttcccgaccctacaagagctggaggcgctcagcagcgttgtgggtgtcaacgcagacaggcttattgagatgctcccttcacagggcatgaccatgaagccccgtcccatccgcctgtgcggggcctgctatgccgagtcgccatgtcaccgaattgaatggcaatgcaaagatcgcatgaagtgcgaccggcataacttacgcctgctcatcaagtgtacaaattgcgagacgccgttccctatcccggcggattgggtgaaggggcaatgtccacattgttccctacccttcgccaaaatggctaagcgtcagcgccgagactag

SEQ ID NO: 32 - DNA, 555 bp, Artificial Sequence
  Synthetic sequence. AcTniQ_pCO3
atggcccagaacatattcctgtcgaagacggagatcggcattgacgaagacgacgagatccgccccaaattggggtacgtcgaaccttatgaggaggagtctattagccattacctcggtcgtttgcggcgcttcaaagcgaactccctgccgagcggctacagcctcggcaagatcgctgggctgggcgcaatgataagcagatgggagaagctgtactttaacccgttcccgactcttcaagaacttgaggcgctcagctccgtggttggcgtcaacgcggatcgtcttatcgaaatgctaccctcccagggcatgacgatgaagccgcgtcccattcgcctctgcggagcttgctacgcggagagcccttgtcatcgcatcgaatggcagtgcaaagaccgtatgaagtgcgaccggcataacctgcgcctgcttattaagtgcacaaattgtgagacgccgttcccgataccggctgattgggtgaagggtcagtgtcctcactgctccctaccattcgcaaagatggctaagcgccagcggcgagattga

SEQ ID NO: 33 - DNA, 555 bp, Artificial Sequence
  Synthetic sequence. AcTniQ_pCO4
atggcccagaacatcttcctaagtaagacagaaatcgggattgatgaagatgacgaaattcgtcctaaactcggatatgtggagccatacgaggaggagagtatatcccattatcttggacgcttgaggcggtttaaagctaactcccttccttctggatactcactcggcaagatagccgggttgggtgcaatgatctcacgctgggagaaactgtacttcaacccgttcccaacactacaggagctggaagctctatcgtccgtagttggtgtaaatgccgacaggcttattgaaatgttacctagtcagggaatgactatgaagcccagaccaattcgcctgtgtggggcctgttacgccgaatcaccatgccacagaattgaatggcaatgtaaggaccgtatgaagtgcgatagacataacttaagactcctcatcaagtgtaccaattgcgaaactcctttccccatacctgctgattgggttaagggccaatgccctcactgttctctcccattcgctaagatggctaagaggcagaggagagattga

SEQ ID NO: 34 - DNA, 1929 bp, Artificial Sequence
  Synthetic sequence. AcCas12k_pCO2
atgtcggtcatcacgatccagtgccgcctcgttgccgaggaggatagtctccgccagctctgggaacttatgtcggaaaaaaatactccgtttatcaacgagatattgctgcaaatcggcaaacacccggagttcgagacttggctggagaagggtcgcattccggccgaactcctcaagactctaggaaactccctcaaaacacaagaacccttcacggggcagccggggaggttctacacgagcgcaataacgttggtcgattacctgtacaagtcgtggttcgccctacagaaacgccgcaagcagcagatcgaggggaagcagcggtggctcaaaatgctcaaatcggatcaggagttggaacaagagtcccagtcgagcctggaggtgatccgcaacaaggcgaccgagctattctcgaaattcacaccccagtccgactcggaggcgctccgccgaaaccagaacgacaagcagaagaaggtcaagaagaccaaaaaatccaccaagccgaaaaccagttcaatcttcaagatattcttgagcacctatgaggaggcggaggagccactcacgcggtgcgcgctggcgtacctccttaagaacaattgtcagatcagcgaactagacgagaaccccgaggagttcactaggaacaagaggcgcaaggagatcgagatcgagcggctcaaggaccaactccagagccgcatcccgaagggtagggacttgaccggggaggagtggctagagaccctggagatcgccaccttcaacgtgccccagaacgaaaacgaggcaaaagcctggcaagctgcattactgcgtaaaacggctaacgtgcccttcccggtggcctacgagtcaaacgaggacatgacctggctcaagaatgacaagaacagattgttcgtgcggttcaacggcctcggaaaactgacgttcgagatatactgcgacaagcggcatctccattacttccagcgcttcctcgaagaccaggaaatactccggaacagtaagcgacagcactcgtctagcctcttcaccctgcgctctggaaggatcgcgtggctgcctggggaggaaaagggcgaacactggaaggtgaatcagctcaacttctattgcagcctggacaccaggatgctcaccacggagggtactcaacaagtggttgaggagaaggtgaccgcaatcacggagatccttaacaaaacgaagcagaaggatgacctgaacgacaagcaacaagccttcataacgaggcagcagtccacacttgcgcgcatcaacaatccattcccacggccctcaaagcccaactaccaggggaagtcctctatcctgatcggggtttccttcgggctggagaagccggtgacggtcgcggtcgttgatgtggttaagaacaaggtgatcgcgtaccgaagcgtgaagcaactcctcggcgagaattacaacctgctcaacagacaacgccagcagcagcagcgcctctcccacgagagacacaaggcgcagaagcagaatgccccaaactcgttcggagagagcgagcttggccaatacgttgacaggctcctagcagacgccatcatcgccatagccaagaagtatcaggcgggctccatcgtactcccgaaactgcgcgacatgcgggagcagattagctctgagattcagtcgcgggctgaaaaccagtgtcccggctacaaggaggggcagcagaagtacgctaaggagtaccggataaacgtccatcgctggtcctacggacggctaatcgagtccatcaagagccaggcggctcaggcgggcattgcaatcgagacgggcaaacaatccatccgaggatcaccacaggagaaggcgcgggatttggctgtgttcacttaccaggagcgccaggccgcgctaatctaa

SEQ ID NO: 35 - DNA, 1929 bp, Artificial Sequence
  Synthetic sequence. AcCas12k_pCO3
atgtccgttatcaccatccaatgccgcctggtggcggaggaggacagcctgaggcagttgtgggaactgatgagcgaaaaaaacaccccgtttatcaatgagatcctgttacaaattgggaagcaccccgagttcgagacctggctggaaaaggggcgcattccagcagaactgctcaaaaccctgggcaactcactcaagacccaggagccgttcacaggccagccgggccggttttacaccagcgccatcacactcgtggactacctgtacaagtcctggttcgccctccaaaagcggcgaaagcagcagatcgagggcaagcagaggtggttgaagatgctgaaaagcgaccaagagcttgaacaagaatcccagtccagtcttgaagtgattcgcaacaaggccaccgagctgttctcgaagttcacgccacagagcgactcagaggcgctgcggcggaaccagaacgacaaacagaagaaggtcaagaaaactaagaaatccaccaagcccaagaccagtagtatcttcaagatattcttgtcaacctacgaggaagccgaggagccccttacccgctgcgccctggcctacctgctcaaaaacaactgccagatttccgagctggacgaaaacccggaggagtttacccggaacaagaggcgaaaggaaatcgaaatcgagcgcctcaaggaccagctccagtctcgtatccctaagggcagagacctgacaggcgaggagtggctggaaaccctggagatcgcaactttcaatgtgcctcagaacgagaacgaggcgaaggcttggcaagcggccctgttgcgtaagacggctaacgttcccttcccagtggcctacgagagcaacgaagatatgacctggctcaagaacgacaagaacaggctgttcgtgaggttcaatggattggggaagctgactttcgagatatactgtgataagcggcatctccattacttccaacggttcttagaggaccaggagatcctgagaaactcaaaacgccagcatagcagctccctgtttaccctgaggagtgggcgcattgcgtggctgccgggtgaggaaaagggtgagcactggaaagtgaaccagcttaacttctactgctccctggacaccaggatgctgacaacggagggcactcaacaagtcgttgaggagaaggttacggccatcaccgagatcctgaacaagacgaagcaaaaggatgacctcaatgacaaacaacaggcgttcattacaagacagcagtcaacattggcgcgtatcaataacccctttcctagaccctctaagccgaactatcagggcaagagcagcatcttgatcggggtgagcttcgggctggagaagcccgtcacggtcgccgtcgttgatgtcgtcaagaataaggtcatcgcttaccgcagcgtgaagcaactcctgggcgagaactacaacctccttaacagacaacggcagcagcagcagcggctgagtcacgaacgccacaaggcacagaagcaaaacgctccgaactcgttcggcgagtccgaactcggacaatacgttgacaggctcttggccgacgcgattatcgcaatcgccaaaaagtaccaggctggctcaatagtgttgcccaaattgagagacatgcgcgagcagataagctcagagatccagagccgcgccgagaatcagtgtccgggatacaaggagggtcagcagaagtacgcgaaggagtatcgtattaacgtgcatcgttggtcctatgggcggcttatcgagtccatcaagagccaggccgcacaggccgggatagcgatagagaccgggaagcagtccatcaggggttcaccacaggagaaggcgcgtgatctcgccgtcttcacgtaccaggaacgccaagccgccttgatctga

SEQ ID NO: 36 - DNA, 1929 bp, Artificial Sequence
  Synthetic sequence. AcCas12k_pCO4
atgtccgttatcaccattcagtgtcggctcgtggccgaggaggacagcttgcgacaattgtgggaactaatgtctgaaaaaaataccccgtttatcaacgaaattctcctccaaatcggaaaacatcctgagtttgagacttggctcgaaaagggccgtatcccggccgagttactaaagaccctcggaaactccctcaagactcaggagcccttcacgggccaaccaggccgcttctacacttctgcgataacgctggtggactacctctacaagagctggttcgctttgcaaaagcggaggaaacaacagattgaaggaaagcagaggtggttgaagatgcttaaatccgatcaggagttggaacaagaatcacagagttcattggaggttatccgcaataaggctacggagctgttcagcaagttcactccacaatcagattcagaggctcttagacgcaaccagaacgataagcagaagaaggtcaaaaagaccaagaagtcaacaaagcctaaaaccagttcaatcttcaaaatcttcctttctacttacgaggaagctgaggagcctttgactcgctgtgcgttggcatacttgctcaaaaacaattgtcagataagcgagttggacgagaaccctgaggagttcactaggaacaagcgacgcaaggaaatcgagatcgaaaggttgaaggatcaactccaaagccgaatacctaaggggagagatttgactggtgaggaatggttggaaacacttgagattgccacgttcaacgttcctcaaaacgaaaacgaggcaaaagcctggcaagctgccctcctgcgaaagaccgcaaacgttcctttcccagtggcctatgagagtaacgaagacatgacatggctcaagaacgacaagaatagacttttcgttcgcttcaacgggcttggcaaattgactttcgaaatctattgcgataagcgacatctgcattacttccagcgtttccttgaggatcaggagatactgagaaactcaaaacggcagcactcctccagcttgttcacacttcgctcaggacgaatcgcttggctgccaggggaggaaaagggagagcattggaaggtcaaccagctcaacttctattgctcacttgacactagaatgcttacaactgaggggacacaacaagtcgtggaggaaaaggttaccgcaattacagagattctaaacaaaacaaaacagaaggacgatttgaatgacaaacaacaagccttcattacaaggcagcaaagtacgcttgcaaggataaacaatccattccctagaccgtccaagcccaactatcagggaaagagttccatcctgattggtgtgagtttcgggctggagaaaccggttaccgtggcagttgtggatgtggtcaagaataaggtgattgcctatcggagtgttaagcaacttctcggtgagaactacaatctactaaacagacaaaggcaacaacagcaacgactctctcacgagagacacaaggctcagaaacagaatgcacccaactcgttcggagagtctgaattggggcaatatgtggaccgcctgcttgctgacgccattatcgccattgccaagaagtatcaagctggcagcattgtacttcctaagctccgggacatgcgggagcagataagcagtgagattcaatcgagggcggaaaatcagtgtccaggctacaaagaggggcagcagaagtatgctaaggaataccgcatcaacgtccatcggtggtcctatggaagactaatcgagagtattaagtctcaagcggctcaagctggcatagccattgagacaggcaagcagagcataagagggtcaccacaggagaaggctagagatttggccgtgttcacttatcaggaaagacaggcagccttaatctga

SEQ ID NO: 37 - DNA, 1755 bp, Artificial Sequence
  Synthetic sequence. ShTnsB_bCO1
atgaactctcaacaaaacccagatctcgcagtgcatcccctagctatccctatggaaggcttattaggcgaaagtgctacaacacttgaaaaaaacgtgatagcaacacaactgtcagaagaagcgcaggttaaattagaagtgatccagtctctgttagagccctgcgatcgtacgacgtacggccagaaactgcgtgaggccgccgagaaattaaatgtgtctttgcggactgtacagcgcctggtaaaaaattgggaacaagatgggttagtaggactgactcaaaccagtcgcgctgataaaggaaaacaccgaataggcgaattttgggaaaattttatcacaaaaacctacaaagaaggaaataaaggaagcaaaaggatgacccctaaacaagtagcccttagagttgaagcaaaggcacgggaactaaaggactcgaaacctccgaactataaaaccgtgctcagagtactggcgccgattctggaaaagcaacaaaaagcgaaaagcattcgcagtccgggttggcgcggtacaaccctttctgttaagacccgagaaggcaaagatttaagtgtcgattattccaatcacgtatggcagtgcgatcatacaagagtggatgtattactggtagaccaacacggtgaaattttaagtagaccttggctcactacagtaattgatacatatagtagatgcattatgggaattaaccttggattcgacgcacccagtagtggagtagttgcattagctttaagacacgcgattttacctaaaagatatggaagcgagtacaagttacattgcgaatggggcacatacggaaaacctgagcatttctacacagatggcggaaaggactttagatcgaaccacttaagccaaattggagcacaactgggatttgtctgccacttgcgcgatagaccaagtgaaggaggagttgtggaaagaccatttaaaaccttgaatgatcagttattctcaactctgccaggatataccggcagcaatgtacaagagcgtccagaagatgccgaaaaagacgctagattaaccttacgagaattagaacagctgctggtgaggtatatcgtcgaccgttacaaccaaagtattgatgctcgtatgggtgatcagacaagatttgaaagatgggaggcgggtctgccgaccgtgcccgtacccattccggaacgcgatctggatatctgtttaatgaagcagagccgtcgaaccgtccaaagaggaggttgcttacaatttcagaacctgatgtaccggggcgaatatttagcgggttatgcaggcgaaacagtgaatttgcgctttgatccccgagatataacaacaattcttgtttatcgacaagaaaacaatcaagaagtttttttgacccgcgcacacgcacagggtcttgaaaccgaacaattagccttagacgaggccgaagccgcttcccgccgtttacgtacggcggggaaaacaattagcaaccaatccttattacaagaagttgttgatcgtgacgccttagttgccaccaaaaagtcgcgcaaagaacgccagaagctagagcagacggtccttcgctcggcagcagtggatgagtccaatcgggagtcactgccaagccaaattgtggaaccagatgaagtcgaaagcaccgaaaccgttcatagtcaatatgaagacattgaagtatgggattatgaacaattacgggaggaatatggcttctaa

SEQ ID NO: 38 - DNA, 828 bp, Artificial Sequence
  Synthetic sequence. ShTnsC_bCO1
atgacagaagcacaagcaatcgcaaaacaattaggaggggtaaaacctgacgacgaatggcttcaagctgaaattgctagacttaagggcaaatctatcgttcctcttcaacaagttaaaacgttacatgactggttagacggaaaaagaaaggcccgcaagtcctgccgcgttgtgggcgaatccagaacaggcaaaacagtggcatgtgatgcctatcgctaccgccacaaaccgcagcaggaggctggaaggcctcccaccgtaccggttgtttacattagaccccatcagaagtgtggacctaaagatctgtttaaaaaaatcacagaatatctgaaatacagagtaactaaaggtacagtgagcgattttcgggatcgaaccatcgaagttttgaaaggatgcggagtcgaaatgcttataatagatgaagcagatcgtctgaagcctgagacattcgccgatgtccgcgacattgcagaagatttaggtatcgccgtcgtcttagtcggcaccgaccgattagatgccgtcataaagcgtgatgaacaagtcttagaacgtttccgtgcgcaccttcgtttcggtaaactgtcaggcgaagattttaagaataccgtagaaatgtgggaacagatggtgctgaaacttcccgtaagtagtaacttaaaaagtaaagagatgctgcgtatactgacctctgctactgaaggatatatcgggcgcctggatgaaatcttacgtgaagcagcaatcagatctctttctcgcggtctaaaaaaaattgataaagccgtattacaggaggttgccaaagaatataag

SEQ ID NO: 39 - DNA, 504 bp, Artificial Sequence
  Synthetic sequence. ShTniQ_bCO1
atgattgaagcacctgatgtgaaaccgtggctatttctgattaaaccttatgaaggcgaaagtctcagtcattttttaggccggtttcgtcgtgcaaaccaccttagcgcatctggacttggaacattagcgggaattggagcgattgtcgctagatgggaacgctttcattttaatccgcgcccgtcccaacaggagttagaagccatcgcctctgtggtagaagtagcagcccaacggttggcacaaatgttgcctcctgcgggggttggtatgcaacatgaacctatcagattatgcggagcttgctatgctgaatctccttgtcatcgcatcgaatggcagtataaaagtgtgtggaaatgtgatcggcatcaactgaaaatattagctaaatgcccaaattgccaagcaccatttaaaatgccggccttatgggaagatgggtgctgccatcgctgtcggatgccatttgccgaaatggcaaaactacaaaaagtttaa

SEQ ID NO: 40 - DNA, 1920 bp, Artificial Sequence
  Synthetic sequence. ShCas12k_bCO1
atgtcacaaattaccatccaagcacgcctaatctcttttgaaagcaatcgtcaacaattatggaaactcatggcggatttaaacacaccgttaatcaacgagctgctttgtcaacttggccaacatccagatttcgagaaatggcagcagaaagggaagttacccagtaccgtcgtaagccaactctgccaaccgctgaagacagatccaagatttgcaggccaaccgtcgcgcttgtacatgagcgcgattcatatagtcgattacatttacaaatcctggttagccattcagaaacgccttcaacaacaattagacggaaaaacacggtggctggagatgttgaattcagatgcggagctcgtggaattaagcggagacacattggaagcaatcagagttaaagccgcggaaattttagcaatagccatgccagccagcgaatctgattcagcctcgcctaaaggcaagaaaggcaagaaagaaaaaaagccgagctccagctcccccaaacggagcctgagcaaaactctgtttgacgcttaccaggaaacagaggatatcaagtctcggagtgcgatttcctacttactgaagaatggttgcaaattaacagataaagaggaggacagtgagaagtttgctaagagacgtcgtcaggtagaaatccaaattcaaagacttaccgaaaaactgatcagcagaatgccgaaaggaagagatttaacaaatgctaaatggcttgagacccttttaacagccacgacaacagtagctgaggataacgcccaagccaaaagatggcaggacattcttttaactcgcagtagcagtttaccgtttccattagtatttgaaacaaatgaagatatggtgtggagtaagaatcaaaagggacgactgtgcgtacactttaacggactgagtgatctcatctttgaggtttactgcggtaacaggcagcttcattggtttcagcgctttttggaagatcagcagactaaaagaaaaagcaagaatcagcacagtagcggattatttacattaagaaatggacaccttgtgtggttagagggcgaagggaaaggagagccctggaatctccatcaccttacattatactgctgcgttgacaacagattatggaccgaagaaggaacagagatcgtacgtcaggagaaagcagacgaaattaccaaatttattacaaatatgaagaagaaaagtgacttatcagatacccaacaagcacttattcaaagaaagcaatcaacattaacccgaatcaataatagtttcgaacgtccatcccagccattatatcagggacagtctcacattttagtaggagtaagcctgggcctcgagaagccggccacggtcgcggtagtggatgcaatcgcgaacaaagtgttggcatatagatccattaaacagctcctgggtgataattatgaactgttaaatcgccaacggagacagcaacagtacctgagccacgagcgccataaggcacaaaaaaatttctcccctaaccaatttggcgcctccgaactgggacaacacatcgatcgcctcttagccaaagccatcgtcgcgcttgcccgcacctataaggccggttcaatcgtgctgcctaagttaggcgatatgcgtgaggtggtccagagcgaaatccaagcaatagcagaacaaaaattcccggggtacattgaaggccaacaaaagtatgctaaacaatatcgagtcaacgtgcataggtggagttatggtcgtctgatacaatcgatccaatctaaagctgcccaaactggcatcgtaattgaggaaggtaaacagccgatacgtgggagccctcacgacaaagccaaggaactggcgctgtccgcctacaatctgcggttaacgcggcggagctga

SEQ ID NO: 41 - DNA, 1866 bp, Artificial Sequence
  Synthetic sequence. AcTnsB_bCO1
atggcagacgaagagttcgaatttacggaaggcacaacacaagtgcctgatgcaatcctcctggacaaaagcaatttcgtagtagatccgtctcaaattattcttgcaacgtcggatcgtcataagctcacatttaatttaatccaatggctggcagaatctcccaatcgcaccatcaagtctcaacgtaaacaggccgtggcaaacacgctggatgtctctactcgccaagtggaacggctgttaaaacaatacgatgaagacaaattgagggaaacagctggcatcgaacgggcagataagggaaaataccgagtgtctgaatactggcagaactttatcacaactatttatgaaaaaagcctgaaggaaaaacatccgatttcaccagcatccattgttcgcgaagttaaacgccatgcaatcgtggatttagaactaaaactcggtgaatatcctcatcaggccactgtttatagaatcctagaccccctcatcgagcagcagaagagaaaaacgcgcgtgcgtaacccgggatcggggtcgtggatgaccgtcgttacacgcgatggcgagctgttgcgagctgatttctcgaatcaaatcattcagtgcgatcacacaaaacttgatgttcgaattgttgataatcatggaaatctgctgtctgatagaccttggctaacaacaattgtcgatacattttccagttgcgtggtaggattccgcttatggattaagcaaccagggtcgacggaagtagcattggcactgcgccacgccatcttaccgaaaaactacccggaagattaccagcttaacaaaagctgggacgtatgcggccacccttatcagtactttttcaccgatggtggaaaagatttccgctcaaagcacttaaaggctatcggtaaaaagttaggcttccaatgcgagttacgcgatcgcccacctgaaggaggaattgttgaacggattttcaagacgattaatactcaagtattgaaggagctgccaggctatacaggtgccaatgtacaagaacgcccagaaaatgcagagaaagaggcttgcctgaccattcaggatcttgataaaattctagcaagttttttctgtgatatttacaatcacgaaccgtatcccaaggaaccacgcgatacacgtttcgagcgctggttcaaaggaatgggaggaaagcttccagaacctttggatgagagagaattagacatttgccttatgaaggaggctcaaagagtggtgcaagcgcatggttcgatccagttcgaaaatctgatctatcgcggagaattccttaaggctcataagggagaatacgtaactttgcgctatgacccagaccatatcctgagtctctatatttacagcggcgagaccgatgataatgcaggcgaattcctcggatatgctcatgctgtgaatatggatacacatgacttatctatagaagaattaaaagccctgaacaaggagcgtagcaatgcgcgcaaggaacatttcaattacgacgcgttacttgccctcggaaagcgaaaagagcttgtggaagagcgcaaagaagataagaaagctaaacgtaactcagagcaaaagcgtctccgttcagcttcgaaaaagaatagtaatgtaatcgagttacgaaaatcacgaacttcaaaatcacttaaaaagcaggaaaatcaggaagtgttaccagagcgcatatccagggaagaaatcaagctcgagaagatcgaacaacaaccacaggaaaatctctcagcaagcccgaatacccaggaagaagaacgccacaagttggtgttctctaatcgccagaagaacctgaataaaatctggtaa

SEQ ID NO: 42 - DNA, 852 bp, Artificial Sequence
  Synthetic sequence. AcTnsC_bCO1
atggcacaaccgcaattagcaacacaaagtatcgtcgaagtcttagcaccccgtttagacattaaggcccagattgcgaaaacaattgacatcgaagaaattttccgtgcttgtttcattacgacagacagagcgtctgagtgttttagatggcttgatgaacttagaattttaaagcaatgcggccgtatcattggtccgcgtaacgtgggtaaatctcgcgccgcgttgcattaccgggatgaagacaaaaagagagtgtcttacgtcaaagcatggtctgcatcttcctcgaaaagactgttttcccagattctgaaagacattaatcatgcagccccgacgggcaaacggcaggatctgcggccgcgtctcgcagggagcttagagctgtttggcttagagttagttataatcgataacgcagaaaacttgcagaaagaagcacttctggatcttaagcaattatttgaggaatgcaatgtccctattgttttagcaggtggtaaggagcttgatgatcttctccacgactgcgatctccttacgaattttcctacgctctatgaatttgaacgccttgaatatgatgatttcaagaaaacactgactacaattgagttagatgtgctgtcattgcccgaagcgtcaaatttagctgagggcaacatcttcgaaattctagccgtctcgaccgaagcacgcatgggcatacttataaaaattctgactaaagcggtcttacatagcctgaagaatggatttcaccgagttgatgaatccattttggagaaaatcgccagtcgctacgggaccaagtacatcccactcaaaaaccggaatcgggactaa

SEQ ID NO: 43 - DNA, 555 bp, Artificial Sequence
  Synthetic sequence. AcTniQ_bCO1
atggcacaaaacatctttctttccaaaacagagatcggtattgacgaagacgacgaaattcggcctaaattaggctatgtcgaaccatacgaagaggagagcattagccattacctgggacgcctgcgccgcttcaaagctaattctttaccgagcggttattctttaggcaagattgccggcttgggcgccatgatttctcgttgggaaaaactctacttcaatccattcccaacattacaagagttggaagctttgagttctgtcgtaggtgttaatgcggatcgtttaattgaaatgttaccctctcaaggtatgaccatgaaaccgcgtcccattcgtttatgcggtgcgtgctatgctgaatcaccatgtcatcgcattgaatggcagtgtaaagatcggatgaagtgtgaccgtcataatctgcgcctgttaatcaagtgcacaaattgcgaaacaccttttccaattcctgcagactgggtcaagggtcaatgcccacattgcagtcttccatttgccaagatggccaaaaggcaacggagggactga

SEQ ID NO: 44 - DNA, 1929 bp, Artificial Sequence
  Synthetic sequence. AcCas12k_bCO1
atgtcagtaataacaatccaatgtagactcgttgcagaagaagattcgctgcgccaattatgggaacttatgtcggaaaaaaacacgccgttcatcaacgaaattctgcttcagattggcaagcaccctgaatttgaaacgtggttagagaaaggacgtattccggcagagctgctcaaaacgctggggaatagcctgaaaacacaggagccgtttacaggccagccaggtcggttctacacaagcgctattacactggtagattacttatacaaaagctggttcgcattgcagaaaagaagaaaacagcaaattgagggcaagcagcggtggctgaagatgctcaaatcggatcaggaattagagcaggagagtcaatctagcttagaggtcattcgcaataaagcgacagagttgttctcaaagttcacaccgcagagcgattctgaagcgttaagacgcaaccagaatgataagcaaaagaaggtcaaaaaaactaaaaaatccaccaagccgaagaccagctcgattttcaagatttttttatcaacatatgaagaagcggaagaacccctgacacgatgtgccctggcctacttattaaaaaacaattgccaaataagtgaattggacgagaatcccgaagagtttacacgcaataaaaggcgtaaagaaattgaaattgaacggctcaaagaccagttacagtctagaatccctaagggcagagatttgacaggagaagagtggctggagacgcttgaaatcgcaacattcaatgtacctcaaaatgaaaatgaagcaaaagcgtggcaagcagcgcttttaagaaaaacagcaaacgttccattcccagtagcttacgaaagtaatgaggacatgacatggctaaagaacgataaaaacagactttttgtacgcttcaatggactgggaaagctcaccttcgagatttactgcgataagcgccacctccactattttcagagatttcttgaggaccaagagatcttgcgcaattcaaaaagacagcattctagcagtttgtttactctgagaagcggcagaattgcttggttgccaggagaggagaaaggagagcactggaaagtgaaccaactgaatttctattgctctttggatactcgcatgctgaccaccgaaggaacacaacaggtcgttgaagagaaagtgacagcaatcacagaaattcttaataagaccaaacaaaaagatgatctcaacgacaaacaacaagcattcatcactcgccaacagtcaacactcgcgcgtattaacaacccatttccacgtcccagtaaaccaaattatcagggtaaatcgtctattctgatcggtgtaagctttggactggaaaagccggtgacagtagcggtggtagatgtagtcaaaaataaagtcatcgcatatcggtccgtcaagcagctcctgggtgaaaattacaatctgttaaaccgtcagcggcagcaacaacaacgcctgagccatgaacgtcataaagcgcagaagcagaatgcgcctaacagtttcggagaaagcgaactgggacaatacgtagatcgcttgttagcagacgcaatcatcgcaatcgcaaaaaaatatcaggcaggtagtatcgtcctccccaaacttcgcgacatgcgcgagcaaatttcttccgagatccaatcgcgggcagagaaccagtgcccgggctataaagaaggccaacaaaagtatgctaaagagtatcgcattaacgtacaccgctggagttatggccggctgatagaatctatcaaatcccaagcggctcaagctggaattgccattgaaaccggcaagcaatcaatacgtggttctccgcaagaaaaggcccgtgatctcgccgtctttacgtatcaagagcggcaggcagcactgatatga

SEQ ID NO: 45 - DNA, 235 bp, Scytonema hofmanii
  ShLE
ttagacatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactga

SEQ ID NO: 46 - DNA, 237 bp, Scytonema hofmanii
  ShRE
gcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtagcaatgctacctc

SEQ ID NO: 47 - DNA, 500 bp, Anabaena cylindrica
  AcLE
ataactagaatttacttgcgttaaccattaaaataaaatggttgcaaaatagaaatgatcatgggagggttgaaaggagtgctgcgatcgaacacatatcaataatccaataatgcccttgcagcaataatttattgtatagtaaatgttaagttcatgcgacattaatttgcgaaaacttagaataattaaattgactctgaaaaacaaccaccacgacattaatttgcgaataacgacactaaattgcgaaaagcgacatttaatttgcgaatgtacatatataatggagaataggagactcgaacccctgacctctgcggtgcgatcgcagcactctaccaactgagctaattccccttgctagtctcaagttactaactcagacagccgtactatatgttaacactcaggcagggtagttttcacctctttttgcaaaaaaacttcctgtacccggtctaattccaaatcagtcagataatctactgtccagttag

SEQ ID NO: 48 - DNA, 500 bp, Anabaena cylindrica
  AcRE
aagactcctcttgttgtaggttgcccaaattaacttagtcatagtctagcagtaattcaagtctcataatatacatctaactaaaaatttggaaaatattattaatctgtgaatatatagcaattcccaagctcatgaaatacaccccacccgtgctgtcgcgcaccgtagccttaccaaggggaggattggggaggggtaattttgtatctaactagagtgagaaaggctattgtacgttcgcaaattaaatgtcgcttttcgcaaattagtgtcgcaactgcttttaaggactaaaacctttattctataagcatcataagctatttacccctacaaaagactaagttacctaattcgcatattgtatgtcgtaaaacctggtttcgcaaattaaatgtcgtttgttaagaatttggtattttgcaaattagatgtcgcatttttgagtaattcatggtacattagtaccttaattattcatgagtgggtttcactct

SEQ ID NO: 49 - DNA, 23 bp, Glycine max
  GmPDSChr18-TS1
gctgcatggaaagacaaggatgg

SEQ ID NO: 50 - DNA, 23 bp, Glycine max
  GmPDSChr18-TS2
gatccttgacactatcaaagcct

SEQ ID NO: 51 - DNA, 23 bp, Glycine max
  GmPDSChr18-TS3
ggtgtatgttcttaggggaagct

SEQ ID NO: 52 - DNA, 23 bp, Glycine max
  GmPDSChr18-TS4
gattgtcactcaattcgggaggc

SEQ ID NO: 53 - DNA, 23 bp, Glycine max
  GmPDSChr18-TS5
ggcaattcaaaacagcagatctt

SEQ ID NO: 54 - DNA, 258 bp, Artificial Sequence
  Synthetic sequence. Encodes ShsgRNA.
  misc_feature (236)..(258): n is a, c, g, or t
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaagnnnnnnnnnnnnnnnnnnnnnnn

SEQ ID NO: 55 - DNA, 293 bp, Artificial Sequence
  Synthetic sequence. Encodes AcsgRNA.
  misc_feature (271)..(293): n is a, c, g, or t
atacaacagcgccgtagttcatgctccttggagtctctgtactatgaaaaatctggcttagtttggcagttggaagactgtcatgctttctgagcctggtagctgcccgcttctgatgctgctgtcgcaagacaggataggtgcgctcccagcaataaggagtaaggcttttagccatagtcgttatttataacgatgtggatttccacagtggtggctactgaatcacccccttcgtcgggggaatcctccaaaagggtgggttgaaagnnnnnnnnnnnnnnnnnnnnnnn

SEQ ID NO: 56 - DNA, 300 bp, Glycine max
  GmU3 promoter
tccctgcgggctagcctgagaaagggtgtttataatcttgtagtgatagaatcaagcatcataagagctttcaaagcctctaatgagtatttctttttctagaaaagatgaatatttgggatcaatgctgctgctgttatgtaggaaacaccgtggcaggaagattgtatttgtatccttgtgcaatactcagaccaattctgttagaaaaatgacacaaaggccgtgatgctagtcccacatcgagtgtttttaacaaaacaggcgcatatatattactggacgctgagacctttggtc

SEQ ID NO: 57 - DNA, 6079 bp, Zea mays
  Zm7 locus
actatactaattcgatgttatatgattttatatttttctctataatttttgttaatcataagatgctctaagtctaaaaagaataagtaagttataatttgagatagcgagggtactgtttaaaggaatgtctctagatggcaaaactaatccgcatgctagcaccagttgtgcaccatatcacgccattctgaactactactttcatataaaaaaatcttgctttgtaagttgataaataattataacttttgccaccacttagagcatctccaacatcaaatcctaaaatagtcttaaaatttgaaatattactgtatttagagcgtttaggttctcaaaagcaatgttgactacaacggtcaggcctacatcgtgctatttaggaaataaatcatattattaaaaataaatggttgaagattaaatggtaatgagaatgtgactctagtatatggggtacaatatttaggacccgagagacggagctctataaatgcttgatgaaattttataaaatatgaccgatgttggaagggtgttttgtggttttaagcccgacttattcgctgcgctttaatctggcggtggcttatacaatattttaagtatgaacaagtcgggctatgcggtatgacggatgtactgcatcgatttctatgcatagtacctctagtttatttcgccatacaatgaaactagggcatcattagttatttttcgtatatatttggttaaatattgatcggctcatccaataaccggaactatactttttcaagagatgcggagagtagaaatctagcgacttcaccagggcccactgatcggtgaggaatgtcatccgtacatagcaagtgcctgtgtggggctagtgatgaatattggtggccgggtcttttccagggagataaagcagtggtggcgggcccacagacctccctacgtgttccatcttccagtccccaccataaagcgctgcacactccgcgagctctcatttgtccacttctccttctcctcctcgtggtctcgcccaacttctcctgcatagctaacccccaggaaccgacatttgggccaacgagaatgcctagtcccacacgtcagtaagagtgaacctttacgtacgtgtcttcgaagtataggctcccttgcactctacgggtgggacagcaggtaggacacgtcgccgcctcgcccctatcctggcgagtggggtaggtcagatgggtccacatggcagcgagcgggatgatgagaggtggggtttgatttttctcgcgtttgcgtgccaggacaaccattggcgcggcgtgcccaatcgggccgcggcctttcacgtgcctaccgctgcggtgagccgccacgtggtggctcgcagccggccaatgatagcgcactgcgcgcctcggcctctccacacgtacgtgtcccgacgcgtgtcccttcgcctccccaggcgctgtattaaagcgccgggcctcaagccgcacccgggtcgtcggcgtcctggtggtggtcgtatcttatctctctcactgccatagtgccatcgtgcgagcggcgagaaagtagaaggaagtgtgtcgtactcgtactcataccgggttggttggtttttgagacgtgtgcggtcgggagaggggatggagagaaagggagggtgctgcctcgcgccgaggtacgccgccaccgcggccgcgcagcaggcaggcgcggcctggcagatggggaggatcatgctcaagttccgcccgattgcgcctaagccggcggccatggcgcccgcgccggcaccggcatcggcaccggtgaccggatcagccgccggtgccgggagagggaagcgaaaggccgcgtgcggaggcggcgggagccggccaaagttgcgatggtgactgcagcgcctgcggcgacggcggcggcgcaagatgtgggcgactgccgggagcactgcgacaaggagaagtcgtcttcgtcgccgtcctcgtcctcgtcggggacgtcatccgtggactcgtcgccgccaccccggccgcagcagcgccagctcgcaacgctgccgctcatgcccgtgacggcggcggaggataaggccgctgcttgtcctgcgacagtggggccggagctcgtgccgtctcaggtggctacggcggcacggccgttggctccgcgcgcgatgcgccccgctgctgctgcagcttacttggtgacggtggaagaggtgaccgcgacgtggcgcgacggcgaggcgccggcctccgcgacgggccacgacgaggccccggcgttcgtgtcggaccagtggggccgcgtcacgttgtggaacgccgcgtttgtccgcgctgcatccgcggacgacggcgacgaggcggcggcgcccgtggtgctcggcggtgccctcccggcgtggggcacctgcgcgggattcacgtgccgcgtccgcgcccggcactggagcgcccggcgcgtcggttcccccgtggtggcgccctgcgatgtgtggcgtctggacgccgccggcagctacctttggcggctggacctgcaggccgcgctcactctcggcggctgcctgtgaaccgtacaccactgtcaagagcaagcaacgaatatgctgtgctactcctctacgtgcggagacgtgttgtattttattctatgagcgttatttactgacgtaaggtatggtttaatcgacggattgctatcctggtcgggcttaattaaggacgttgaagcctggtaaggtatactaacttagcgtccttcggaatgggagtgagttgggaggacttgaaaatgtaagaagaagaaaacaaagcatgtgatggatgcttctggatgtgcaagctcagtcttcattcttcagcagcgcggaaattggctctaataacattggtttatgttgacagtgctagcgaggacaatgagtcattcgatgtgaaataataccatctgttctttatgatagtctatttagtataatatgatggcatgccctcttaggcagtagccggccagcatttgaaacatggtacttcgttttatttaaaaaataataagttattatttattatttatttgttttatcatttaaggtagtttatatttaacttaaaatttatatttttttaataaatattttgaataatataactggtcaaagtttttgataaaaaatcaacgatgtcatatatttatgaaagaagatagtatttaaaattactaaggactaatttgaaagctccattgttttaagaaatttttattttccttataaaatgaacaaatttttcttaaaaatgcggttcccaaattaactctaagagataaaataattagcatatataatatctataaagtcttaacatattcatattcacgtatcaactactttataactggtaaaaaatggaattattttttattttttatagaaatcttgggtattcatttgaatatgcttgtcactatattgggcccgcctctcctcttggggcctcttggtccattttttatagtctaaggaggagtgcggttacatgtttaagtcagttacatgacacaagtctaggtcggttaagagatataaacagtaaccaacctaaactaagcaagtatagatcagttaagagatatgaacagtaagaagcctagacttagcctatcctgaattatcagggatcatatacttatgggtgcatgaattgctatatttataagataccagtcttatttcccaatctggtcagatcttgccatcttcattatcttaaggtttccttgtcgatgaggacaacttgttacatgggctaagtccctggattactccccatgctccagggatgacttgtcagtcttctttattcgatgattgaaggataatactagtatatttctttaacatagcccctctcaagtcttatttgactaggttattagctcagagagataagacttcgcttgattcttctttcatagcttgattgtgctacaaccaagttgttctaccttttagctgagtccccattgtcgtctcttagcccccaagcccttgaactcgagatttcactaatgaagtcttggttagtttatctgtaggggccaatgttgccaaaggtactttgactagttacatcacatgatcaccttaaaggtgtggggaaaagctatgtcgaagatcactcatttaaactttttactttggcacaaattagtgtatttcaggaatctctactgatggacgaagcgaatcttcggacagtaaggcatgcagagaagcttctcacatacatgcgagtggaagaatgttagccaaaactgatagctttggcgacaaaagaaaggctttgggtgcgagccaaaggagaaggtgacgaagtcttgaacatgcgcaaccgaagctgaagaaaatatgctacttccctaatggcatttgtaaacagtgaagggcacaattgtaatcctatattaggtcggtttgtctcccctataaatagatgaacaatatcccatataaagtaccttgaaccaggtgctacagcttcgtctagatcaacctctgaaacaccttcgtgctatcttgcatcaagaagccgaaggtatgattgtaaacttgtatgagaagagaaataaaatgataaggcacatgagatgagttctcatatcttcgtcttacttcgtgtattccattcaattatactctaccttagtccttgaatcgttatctcgaagagataacactccaaggacgaaggtgtctactttttaacattgtgttgccttgtttttgattccatacgacaattgaaaacaagtgaccaacattaccagactatcactttagattctgaaaaattgagtcgctttagcttcctcatcagcttcagaatagatctttgtgcttctgcattcgcagattcatggcgcttcatcttcggtaatgggctcaaataggactccttcgcaattttagactttcaccatatcatcaatcaataatttagctttccaacgctcccttacatccgtcactttagcttccagtcgcacaaacccttgctttcctccaccgccactagatcgtgagatggtatgaatgaccgcttctcacccacataagtttgaagccttcatattcaatgcgaaagacctaaggaccctagtgacagcaggtctacatcaagaaaagaaaacattcaatggaaaatcttgagtaaggaatccttcccaatgtctgacaatggccaaatcgtcgtttttactcatcatctgaagcacaggatcgatctacatgtctctcacttcttctgtgacctccttcattattatcatattgaattgcttcatcttaatcccaactcgattctgcagattgccattttgtggaattcctcccttcttccctgtaacaccccatacactctcggaccgacggtgcttactcctggcaaccctctaggatcatagaacatccctacaaaccaacacgagccttttacgtgcactttgtcctcactcatgcgtatcggggaaaactttccggttggtcacctatcccaaaattgctctgagccaagcacacttaacccagaggttcttttgagataggcttcaccttgttgatatgagtattctattaattctattaagccttgggccaagaaatcacaatccaccccccttagaagactgatgtctttgtcagtcaaccccaagctagaaacctgccttttggccacgtctgtgtctactgtcatcatatgccatgccatgtgaccactctaggcccacatgcaccatatactcaaacctctagcccacacatgctcgtgaaaccgtgaggttcagctctaatcccatttgtaacaccccctccactcccagactagcgttgcttactcctggcaaccctctaggatcatagaccatccccacaaaccaacacgagtctttttctcacactttgtcctcacacatgcgcaccggggaaaacttctcggttggttacccatcccaaattggtctgagccaagcacgcttaactcagaggttcttttgagataggcttctg

SEQ ID NO: 58 - DNA, 23 bp, Zea mays
  Zm7_TS1
ctagcgaggacaatgagtcattc

SEQ ID NO: 59 - DNA, 23 bp, Zea mays
  Zm7_TS2
agttgggaggacttgaaaatgta

SEQ ID NO: 60 - DNA, 23 bp, Zea mays
  Zm7_TS3
tacggttcacaggcagccgccga

SEQ ID NO: 61 - DNA, 23 bp, Zea mays
  Zm7_TS1
tcaaatgctggccggctactgcc

SEQ ID NO: 62 - DNA, 23 bp, Zea mays
  Zm7_TS2
ctttatgatagtctatttagtat

SEQ ID NO: 63 - DNA, 23 bp, Zea mays
  Zm7_TS3
tatgttgacagtgctagcgagga

SEQ ID NO: 64 - DNA, 23 bp, Zea mays
  Zm7_TS4
atttactgacgtaaggtatggtt

SEQ ID NO: 65 - DNA, 23 bp, Zea mays
  Zm7_TS5
gcttgctcttgacagtggtgtac

SEQ ID NO: 66 - DNA, 23 bp, Zea mays
  Zm7_TS6
cacaggcagccgccgagagtgag

SEQ ID NO: 67 - protein, 337 aa, Artificial Sequence
  Synthetic sequence. TALE
SIVAQLSRRDPALAALTNDHLVALACLGGRPALDAVKKGLPHAPEFIRRVNRRIAERTSHRVADYAHVVRVLEFFQCHSHPAHAFDEAMTQFGMSRHGLVQLFRRVGVTEFEARYGTLPPASQRWDRILQASGMKRAKPSPTSAQTPDQTSLHAFADSLERDLDAPSPMHEGDQTRASSRKRSRSDRAVTGPSAQQAVEVRVPEQRDALHLPLSWRVKRPRTRIWGGLPDPGTPIAADLAASSTVMWEQDAAPFAGAADDFPAFNEEELAWLMELLPQSGSVGGTIAADLAASSTVMWEQDAAPFAGAADDFPAFNEEELAWLMELLPQSGSVGGTI

SEQ ID NO: 68 - RNA, 235 nt, Artificial Sequence
  Synthetic sequence. ShsgRNA
auauuaauagcgccgcaauucaugcugcuugcagccucugaauuuuguuaaaugaggguuaguuugacuguauaaauacagucuugcuuucugacccugguagcugcucacccugaugcugcugucaauagacaggauaggugcgcucccagcaauaagggcgcggauguacugcuguaguggcuacugaaucacccccgaucaagggggaacccuccaaaagguggguugaaag

SEQ ID NO: 69 - RNA, 43 nt, Artificial Sequence
  Synthetic Construct
uagcgccgcaauucaugcugcuugcagccucugaauuuuguua

SEQ ID NO: 70 - RNA, 44 nt, Artificial Sequence
  Synthetic Construct
aggguuaguuugacuguauaaauacagucuugcuuucugacccu

SEQ ID NO: 71 - RNA, 89 nt, Artificial Sequence
  Synthetic Construct
gguagcugcucacccugaugcugcugucaauagacaggauaggugcgcucccagcaauaagggccccgauguacugcguaguggcuacu

SEQ ID NO: 72 - RNA, 16 nt, Artificial Sequence
  Synthetic Construct
cccccgaucaaggggg

SEQ ID NO: 73 - RNA, 20 nt, Artificial Sequence
  Synthetic Construct
aacccuccaaaagguggguu

(19) United States
(12) Patent Application Publication
(10) Pub. No.: US 2022/0348942 A1
(43) Pub. Date: Nov. 3, 2022

(54) CAST-MEDIATED DNA TARGETING IN PLANTS

(71) Applicant: Monsanto Technology LLC, St. Louis, MO (US)

(72) Inventors: Larry GILBERTSON, St. Louis, MO (US); Ervin NAGY, St. Louis, MO (US); Thomas REAM, St. Louis, MO (US); Linda RYMARQUIS, St. Louis, MO (US); Xudong YE, St. Louis, MO (US)

(73) Assignee: Monsanto Technology LLC, St. Louis, MO (US)

(21) Appl. No.: 17/633,557

(22) PCT Filed: Aug. 5, 2020
(86) PCT No.: PCT/US2020/045012

§ 371 Date: Feb. 7, 2022

Related U.S. Application Data
(60) Provisional application No. 62/883,933, filed on Aug. 7, 2019.

(51) Int. Cl. C12N 15/82

(52) U.S. Cl. CPC C12N 15/8213

(57) ABSTRACT

The present disclosure relates to compositions and methods related to using the CAST system to provide targeted transposition of desired sequences into plant genomes. Several embodiments relate to a method for producing a megalocus on a plant chromosome comprising: (a) obtaining a plant comprising a first locus, wherein the first locus comprises an endogenous trait locus or a transgene; (b) providing to the plant tnsB, tnsC, tniQ, Cast 2k, a guide nucleic acid and a donor cassette; and (c) selecting a progeny plant produced from step (b) wherein targeted transposition of the donor cassette has occurred at a second locus targeted by the guide nucleic acid, wherein the first and second locus are genetically linked but physically separate.

### CROSS-REFERENCE TO RELATED APPLICATION

This application claims the benefit of U.S. Provisional Application No. 62/883,933, filed Aug. 7, 2019, which is incorporated by reference in its entirety herein.

### INCORPORATION OF SEQUENCE LISTING

A sequence listing contained in the file named “P34780WO00_SL.TXT” which is 99,319 bytes (measured in MS-Windows®) and created on Aug. 5, 2020, is filed electronically herewith and incorporated by reference in its entirety.

### FIELD

The present disclosure relates to compositions and methods related to using the CAST system to provide targeted transposition of desired sequences into plant genomes.

### BACKGROUND

Systems comprising CRISPR associated proteins, such as Cas9 and Cas12a, and their guide RNAs have been utilized to create genetic diversity in plant genomes by creating targeted double-strand breaks, which are inaccurately repaired by the plant's DNA repair machinery, or by targeting, through tethering to a CRISPR associated protein, cytidine and adenine deaminases. These systems have also been utilized to promote targeted insertion of donor DNAs at the site of a CRISPR-generated double-strand break through either homologous recombination or non-homologous end joining, however, CRISPR-mediated targeted DNA integration is inefficient in plants. CRISPR associated transposases (CAST), which are comprised of Tn7-like transposase subunits, tnsB, tnsC, and tniQ, and the Type V-K CRISPR effector, Cas12k, catalyzes site-directed DNA transposition. Cas12k forms a complex with partially complementary non-coding RNA species, crRNA and tracrRNA and the tripartite ribonucleo-protein (RNP) complex recognizes chromosomal sites for transposition based on the presence of a protospacer adjacent motif (PAM) and complementarity between the variable portion of crRNA and the target DNA. The associated transposases, tnsB, tnsC and tniQ recognize the transposon by the conserved ‘left end’ (LE) and ‘right end’ (RE) boundaries and they insert it into a chromosomal site near the target sequence recognized by Cas12k, preferentially between a TA dinucleotide. Two homologous CAST systems, native in the cyanobacteria species Scytonema hofmanni (UTEX B 2349) and Anabaena cylindrica (PCC 7122) have been demonstrated to be functional for transposition (see Strecker et al., Science10.1126/science.aax9181, 2019) in E. coli.

A CAST system functional in plant cells is needed to promote efficient targeted insertion of donor DNAs at desired location in the plant genome.

### SUMMARY

Described herein are methods and compositions to utilize CAST systems for targeted genome modification in plants. Several embodiments relate to a method for producing a megalocus on a plant chromosome comprising: (a) obtaining a plant comprising a first locus, wherein the first locus comprises an endogenous trait locus or a transgene; (b) providing to the plant tnsB, tnsC, tniQ, Cas12k, a guide nucleic acid and a donor cassette; and (c) selecting a progeny plant produced from step (b) wherein targeted transposition of the donor cassette has occurred at a second locus targeted by the guide nucleic acid, wherein the first and second locus are genetically linked but physically separate. In some embodiments, the first and second locus are located about 0.1 cM to about 20 cM apart from each other. In some embodiments, the first and second locus are located about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 cM apart from each other. In some embodiments, the plant comprises one or more expression cassettes encoding one or more proteins selected from the group consisting of tnsB, tnsC, tniQ, and Cas12k. In some embodiments, the plant comprises one or more expression cassettes encoding one or more guide nucleic acids. In some embodiments, one or more guide nucleic acids are not complementary to a target site in the plant. In some embodiments, one or more of tnsB, tnsC, tniQ, Cas12k, a guide nucleic acid and a donor cassette are provided to the plant by particle bombardment.

Several embodiments relate to a plant, seed or plant part comprising a megalocus produced by (a) obtaining a plant comprising a first locus, wherein the first locus comprises an endogenous trait locus or a transgene; (b) providing to the plant tnsB, tnsC, tniQ, Cas12k, a guide nucleic acid and a donor cassette; and (c) selecting the progeny plant, seed or plant part produced from step (b) wherein targeted transposition of the donor cassette has occurred at a second locus targeted by the guide nucleic acid, wherein the first and second locus are genetically linked but physically separate. In some embodiments, the first and second locus are located about 0.1 cM to about 20 cM apart from each other. In some embodiments, the first and second locus are located about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 cM apart from each other. In some embodiments, the progeny plant, seed or plant part comprises one or more expression cassettes encoding one or more proteins selected from the group consisting of tnsB, tnsC, tniQ, and Cas12k. In some embodiments, the progeny plant, seed or plant part comprises one or more expression cassettes encoding one or more guide nucleic acids. In some embodiments, one or more guide nucleic acids are not complementary to a target site in the progeny plant, seed or plant part. In some embodiments, one or more of tnsB, tnsC, tniQ, Cas12k, a guide nucleic acid and a donor cassette are provided to the plant by particle bombardment.

Several embodiments relate to a T-DNA comprising: a.) a first expression cassette encoding a ShTnsB protein comprising a DNA sequence with at least 90%, 95%, 96%, 97%, 98%, 99% or 100% sequence identity to any of SEQ ID NOs:1, 2, 13-15; b.) a second expression cassette encoding a ShTnsC protein comprising a DNA sequence with at least 90%, 95%, 96%, 97%, 98%, 99% or 100% sequence identity to any of SEQ ID NOs: 3, 4, 16-18; and c.) a third expression cassette encoding a ShTnsQ protein comprising a DNA sequence with at least 90%, 95%, 96%, 97%, 98%, 99% or 100% sequence identity to any of SEQ ID NOs:5, 6, 19-21. In some embodiments, the T-DNA further comprises a fourth expression cassette encoding a ShCas12k protein comprising a DNA sequence with at least 90%, 95%, 96%, 97%, 98%, 99% or 100% sequence identity to any of SEQ ID NOs:7, 8, 22-24. In some embodiments, the T-DNA further comprises a fifth expression cassette encoding a guide nucleic acid. In some embodiments, the expression cassette comprises a DNA sequence with at least 90%, 95%, 96%, 97%, 98%, 99% or 100% sequence identity to SEQ ID NO: 54. In some embodiments, the T-DNA further comprises a pair of recombinase recognition sequences flanking the expression cassettes encoding CAST system components. In some embodiments, the T-DNA further comprises a pair of recombinase recognition sequences flanking the expression cassettes encoding CAST system components, wherein the recombinase recognition sequences are selected from the group consisting of LoxP, Lox.TATA-R9, FRT, RS, and GIX. In some embodiments, the T-DNA further comprises an expression cassette encoding a site-specific recombinase. In some embodiments, the T-DNA further comprises an expression cassette encoding a site-specific recombinase selected from the group consisting of Cre-recombinase, Flp-recombinase, and R-recombinase. In some embodiments, the T-DNA further comprises a donor cassette and wherein the donor cassette disrupts the expression cassette encoding the site-specific recombinase.

Several embodiments relate to a plant comprising the T-DNA a T-DNA comprising: a.) a first expression cassette encoding a ShTnsB protein comprising a DNA sequence with at least 90%, 95%, 96%, 97%, 98%, 99% or 100% sequence identity to any of SEQ ID NOs:1, 2, 13-15; b.) a second expression cassette encoding a ShTnsC protein comprising a DNA sequence with at least 90%, 95%, 96%, 97%, 98%, 99% or 100% sequence identity to any of SEQ ID NOs: 3, 4, 16-18; and c.) a third expression cassette encoding a ShTnsQ protein comprising a DNA sequence with at least 90%, 95%, 96%, 97%, 98%, 99% or 100% sequence identity to any of SEQ ID NOs:5, 6, 19-21. In some embodiments, the T-DNA further comprises a fourth expression cassette encoding a ShCas12k protein comprising a DNA sequence with at least 90%, 95%, 96%, 97%, 98%, 99% or 100% sequence identity to any of SEQ ID NOs:7, 8, 22-24. In some embodiments, the T-DNA further comprises a fifth expression cassette encoding a guide nucleic acid. In some embodiments, the expression cassette comprises a DNA sequence with at least 90%, 95%, 96%, 97%, 98%, 99% or 100% sequence identity to SEQ ID NO: 54. In some embodiments, the plant further comprises a donor cassette. In some embodiments, the plant comprises a donor cassette comprising a DNA sequence with at least 90%, 95%, 96%, 97%, 98%, 99% or 100% sequence identity to SEQ ID NO: 45 and a DNA sequence with at least 90%, 95%, 96%, 97%, 98%, 99% or 100% sequence identity to SEQ ID NO: 46.

Several embodiments relate to Agrobacterium tumefaciens bacterium comprising a T-DNA comprising: a.) a first expression cassette encoding a ShTnsB protein comprising a DNA sequence with at least 90%, 95%, 96%, 97%, 98%, 99% or 100% sequence identity to any of SEQ ID NOs:1, 2, 13-15; b.) a second expression cassette encoding a ShTnsC protein comprising a DNA sequence with at least 90%, 95%, 96%, 97%, 98%, 99% or 100% sequence identity to any of SEQ ID NOs: 3, 4, 16-18; and c.) a third expression cassette encoding a ShTnsQ protein comprising a DNA sequence with at least 90%, 95%, 96%, 97%, 98%, 99% or 100% sequence identity to any of SEQ ID NOs:5, 6, 19-21. In some embodiments, the T-DNA further comprises a fourth expression cassette encoding a ShCas12k protein comprising a DNA sequence with at least 90%, 95%, 96%, 97%, 98%, 99% or 100% sequence identity to any of SEQ ID NOs:7, 8, 22-24. In some embodiments, the T-DNA further comprises a fifth expression cassette encoding a guide nucleic acid. In some embodiments, the expression cassette comprises a DNA sequence with at least 90%, 95%, 96%, 97%, 98%, 99% or 100% sequence identity to SEQ ID NO: 54. In some embodiments, the T-DNA further comprises a pair of recombinase recognition sequences flanking the expression cassettes encoding CAST system components. In some embodiments, the T-DNA further comprises a pair of recombinase recognition sequences flanking the expression cassettes encoding CAST system components, wherein the recombinase recognition sequences are selected from the group consisting of LoxP, Lox.TATA-R9, FRT, RS, and GIX. In some embodiments, the T-DNA further comprises an expression cassette encoding a site-specific recombinase. In some embodiments, the T-DNA further comprises an expression cassette encoding a site-specific recombinase selected from the group consisting of Cre-recombinase, Flp-recombinase, and R-recombinase. In some embodiments, the T-DNA further comprises a donor cassette and wherein the donor cassette disrupts the expression cassette encoding the site-specific recombinase.

Several embodiments relate to a T-DNA comprising: a.) a first expression cassette encoding a AcTnsB protein comprising a DNA sequence with at least 90%, 95%, 96%, 97%, 98%, 99% or 100% sequence identity to any of SEQ ID NOs:9, 25-27; b.) a second expression cassette encoding a AcTnsC protein comprising a DNA sequence with at least 90%, 95%, 96%, 97%, 98%, 99% or 100% sequence identity to any of SEQ ID NOs: 10, 28-30; and c.) a third expression cassette encoding a AcTnsQ protein comprising a DNA sequence with at least 90%, 95%, 96%, 97%, 98%, 99% or 100% sequence identity to any of SEQ ID NOs:11, 31-33. In some embodiments, the T-DNA further comprises a fourth expression cassette encoding a AcCas12k protein comprising a DNA sequence with at least 90%, 95%, 96%, 97%, 98%, 99% or 100% sequence identity to any of SEQ ID NOs:12, 34-36. In some embodiments, the T-DNA further comprises an expression cassette encoding a guide nucleic acid. In some embodiments, the expression cassette comprises a DNA sequence with at least 90%, 95%, 96%, 97%, 98%, 99% or 100% sequence identity to SEQ ID NO: 55. 29. In some embodiments, the T-DNA further comprises a pair of recombinase recognition sequences flanking the expression cassettes encoding CAST system components. In some embodiments, the T-DNA further comprises a pair of recombinase recognition sequences flanking the expression cassettes encoding CAST system components, wherein the recombinase recognition sequences are selected from the group consisting of LoxP, Lox.TATA-R9, FRT, RS, and GIX. In some embodiments, the T-DNA further comprises an expression cassette encoding a site-specific recombinase. In some embodiments, the T-DNA further comprises a pair of recombinase recognition sequences flanking the expression cassettes encoding CAST system components, wherein the site-specific recombinase is selected from the group consisting of Cre-recombinase, Flp-recombinase, and R-recombinase. In some embodiments, the T-DNA further comprises a donor cassette and wherein the donor cassette disrupts the expression cassette encoding the site-specific recombinase.

Several embodiments relate to a plant comprising a T-DNA comprising: a.) a first expression cassette encoding a AcTnsB protein comprising a DNA sequence with at least 90%, 95%, 96%, 97%, 98%, 99% or 100% sequence identity to any of SEQ ID NOs:9, 25-27; b.) a second expression cassette encoding a AcTnsC protein comprising a DNA sequence with at least 90%, 95%, 96%, 97%, 98%, 99% or 100% sequence identity to any of SEQ ID NOs: 10, 28-30; and c.) a third expression cassette encoding a AcTnsQ protein comprising a DNA sequence with at least 90%, 95%, 96%, 97%, 98%, 99% or 100% sequence identity to any of SEQ ID NOs:11, 31-33. In some embodiments, the T-DNA further comprises a fourth expression cassette encoding a AcCas12k protein comprising a DNA sequence with at least 90%, 95%, 96%, 97%, 98%, 99% or 100% sequence identity to any of SEQ ID NOs:12, 34-36. In some embodiments, the T-DNA further comprises an expression cassette encoding a guide nucleic acid. In some embodiments, the expression cassette comprises a DNA sequence with at least 90%, 95%, 96%, 97%, 98%, 99% or 100% sequence identity to SEQ ID NO: 55. In some embodiments, the plant further comprises a donor cassette. In some embodiments, the plant further comprises a donor cassette comprising a DNA sequence with at least 90%, 95%, 96%, 97%, 98%, 99% or 100% sequence identity to SEQ ID NO: 47 and a DNA sequence with at least 90%, 95%, 96%, 97%, 98%, 99% or 100% sequence identity to SEQ ID NO: 48.

Several embodiments relate to an Agrobacterium tumefaciens bacterium comprising a T-DNA comprising: a.) a first expression cassette encoding a AcTnsB protein comprising a DNA sequence with at least 90%, 95%, 96%, 97%, 98%, 99% or 100% sequence identity to any of SEQ ID NOs:9, 25-27; b.) a second expression cassette encoding a AcTnsC protein comprising a DNA sequence with at least 90%, 95%, 96%, 97%, 98%, 99% or 100% sequence identity to any of SEQ ID NOs: 10, 28-30; and c.) a third expression cassette encoding a AcTnsQ protein comprising a DNA sequence with at least 90%, 95%, 96%, 97%, 98%, 99% or 100% sequence identity to any of SEQ ID NOs:11, 31-33. In some embodiments, the T-DNA further comprises a fourth expression cassette encoding a AcCas12k protein comprising a DNA sequence with at least 90%, 95%, 96%, 97%, 98%, 99% or 100% sequence identity to any of SEQ ID NOs:12, 34-36. In some embodiments, the T-DNA further comprises an expression cassette encoding a guide nucleic acid. In some embodiments, the expression cassette comprises a DNA sequence with at least 90%, 95%, 96%, 97%, 98%, 99% or 100% sequence identity to SEQ ID NO: 55. 29. In some embodiments, the T-DNA further comprises a pair of recombinase recognition sequences flanking the expression cassettes encoding CAST system components. In some embodiments, the T-DNA further comprises a pair of recombinase recognition sequences flanking the expression cassettes encoding CAST system components, wherein the recombinase recognition sequences are selected from the group consisting of LoxP, Lox.TATA-R9, FRT, RS, and GIX. In some embodiments, the T-DNA further comprises an expression cassette encoding a site-specific recombinase. In some embodiments, the T-DNA further comprises a pair of recombinase recognition sequences flanking the expression cassettes encoding CAST system components, wherein the site-specific recombinase is selected from the group consisting of Cre-recombinase, Flp-recombinase, and R-recombinase. In some embodiments, the T-DNA further comprises a donor cassette and wherein the donor cassette disrupts the expression cassette encoding the site-specific recombinase.

Several embodiments relate to a method of generating a targeted transposition of a sequence of interest in the genome of a plant cell comprising providing to the plant cell a CAST system, wherein the CAST system comprises: tnsB; tnsC; tniQ; Cas12k; a guide nucleic acid; and a donor cassette, wherein the CAST system transposes the sequence of interest into a target site recognized by the guide nucleic acid in the plant genome. In some embodiments, a plant comprising a CAST system comprises: tnsB; tnsC; tniQ; Cas12k; a guide nucleic acid; and a donor cassette is crossed to a haploid inducer plant to a plant comprising a target site recognized by the guide nucleic acid.

### DESCRIPTION OF FIGURES

FIG. 1: Schematic of expression cassettes designed to test the ShCAST and AcCAST systems in soy protoplasts. (A) Design of expression cassettes encoding ShCAST or AcCAST proteins. pCO=plant codon optimized. NLS=Nuclear localization signal. (B) Design of expression cassette encoding single piece guide RNAs for ShCAST or AcCAST systems. (C) Schematic of a donor cassette comprising transposons carrying a sequence of interest (for eg: selectable marker) flanked by Sh or Ac Left end (LE) or Right end (RE) sequences. (D) Schematic of cassette for expression and purification of ShCAST or AcCAST proteins from bacteria for ribonucleoprotein (RNP) based delivery of CAST system into plant cells. bCO=codon optimized for expression in bacteria.

FIG. 2: Schematic illustrating primers specific to the target region (P1) and the transposon (P2) for detection of targeted transpositions by ‘flank PCR’.

FIG. 3: Schematic illustrating configurations of Agrobacterium T-DNA vectors comprising plant optimized Ac or Sh CAST expression cassettes for delivery of CAST proteins, CAST sgRNA and donor cassette into plants for site directed integration of donor cassette into the genome. TnsB, TnsC, TniQ and Cas12K comprise nucleus localization signal peptide sequences at either or both ends. The donor cassette comprises an SOI (Sequence of interest) flanked by conserved Sh or Ac LE and RE sequences. LB and RB indicate the left border and Right border sequences of the T-DNA. P indicates Promoter. IRES indicates Internal ribosome entry site.

FIG. 4. Schematic illustrating a fused sgRNA for ShCas12a.

FIG. 5. Schematic illustrating configurations of Agrobacterium T-DNA vector designed to inactivate transposase activity. Excision of the donor cassette results in expression of Cre which excises sequence (Pro-tnsB; Pro-tns-C; Pro-tni-Q; Pro-Cre) flanked by lox sites. LB and RB indicate the left border and Right border sequences of the T-DNA. Pro=Promoter; GOI=Gene of Interest; LE=Left End; RE=Right End.

FIG. 6. Schematic illustrating configurations of Agrobacterium T-DNA vector designed to inactivate transposase activity. Excision of the donor cassette results in creation of an RNAi construct for silencing the tniQ component of the CAST system. LB and RB indicate the left border and Right border sequences of the T-DNA. Pro=Promoter; GOI=Gene of Interest; LE=Left End; RE=Right End.

FIG. 7. Schematic of expression cassettes designed to inactivate transposase activity. Design of expression cassettes encoding ShCAST or AcCAST proteins. LTR=Long Terminal Repeat; SINE=Short Interspersed Nuclear Elements; HelEnds=conserved terminal repeats of Helitrons; ITR=Inverted Terminal Repeats.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

Any composition, nucleic acid molecule, polypeptide, cell, plant, etc. provided herein is specifically envisioned for use with any method provided herein.

Several embodiments described herein relate to methods and compositions for utilizing CRISPR associated transposase (CAST) systems derived from Scytonema hofmanni (ShCAST) and Anabaena cylindrica (AcCAST) in plant cells. The methods provided may be executed in various cell, tissue, and developmental types, including gametes of plants. It is further anticipated that one or more of the elements described herein may be combined with use of promoters specific to particular plant cells, tissues, parts and/or developmental stages, such as a meiosis-specific promoter.

Several embodiments relate to using a ShCAST system comprising the Tn7-like transposase subunits, tnsB, tnsC, and tniQ, and the Type V-K CRISPR effector, Cas12k to perform targeted insertion of a sequence of interest in plant cells. In some embodiments, the ShCAST system further comprises a crRNA and tracrRNA. In some embodiments, the ShCAST system further comprises a guide nucleic acid comprising a nucleotide sequence as set forth in SEQ ID NO: 54. In some embodiments, the ShCAST system further comprises a donor cassette comprising a sequence of interest flanked by a left end boundary sequence (LE) and a right end boundary sequence (RE). In some embodiments, the ShCAST system further comprises a donor cassette comprising one or more expression cassettes flanked by a nucleotide sequence as set forth in SEQ ID NO: 45 and a nucleotide sequence as set forth in SEQ ID NO: 46.

Several embodiments relate to using an AcCAST system comprising the Tn7-like transposase subunits, tnsB, tnsC, and tniQ, and the Type V-K CRISPR effector, Cas12k to perform targeted insertion of a sequence of interest in plant cells. In some embodiments, the AcCAST system further comprises a crRNA and tracrRNA. In some embodiments, the AcCAST system further comprises a guide nucleic acid comprising a nucleotide sequence as set forth in SEQ ID NO: 55. In some embodiments, the AcCAST system further comprises a donor cassette comprising a sequence of interest flanked by a left end boundary sequence (LE) and a right end boundary sequence (RE). In some embodiments, the AcCAST system further comprises a donor cassette comprising one or more expression cassettes flanked by a nucleotide sequence as set forth in SEQ ID NO: 47 and a nucleotide sequence as set forth in SEQ ID NO: 48.

Methods are known in the art for assembling and introducing constructs into a cell in such a manner that the transcribable DNA molecule is transcribed into a functional mRNA molecule that is translated and expressed as a protein. For the practice of the invention, conventional compositions and methods for preparing and using constructs and host cells are well known to one skilled in the art. Typical vectors useful for expression of nucleic acids in higher plants are well known in the art and include vectors derived from the Ti plasmid of Agrobacterium tumefaciens and the pCaMVCN transfer control vector.

Several embodiments relate to a AcCAST system that is optimized for expression in plant cells. As used herein, “codon optimization” refers to a process of modifying a nucleic acid sequence for enhanced expression in a host cell of interest by replacing at least one codon (e.g., at least 1, 2, 3, 4, 5, 10, 15, 20, 25, 50, or more codons) of a sequence with codons that are more frequently or most frequently used in the genes of the host cell while maintaining the original amino acid sequence. Various species exhibit bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www(dot)kazusa(dot)or(dot)jp/codon and these tables can be adapted in a number of ways. See Nakamura et al., 2000, Nucl. Acids Res. 28:292. Computer algorithms for codon optimizing a particular sequence for expression in a particular host cell are also available, such as Gene Forge (Aptagen; Jacobus, Pa.), are also available. As to codon usage in plants, including algae, reference is made to Campbell and Gown, 1990, Plant Physiol., 92: 1-11; and Murray et al., 1989, Nucleic Acids Res., 17:477-98. In some embodiments, a nucleic acid encoding a CAST system component is codon optimized for a corn cell. In another aspect, a nucleic acid encoding a CAST system component is codon optimized for a rice cell. In another aspect, a nucleic acid encoding a CAST system component is codon optimized for a wheat cell. In another aspect, a nucleic acid encoding a CAST system component is codon optimized for a soybean cell. In another aspect, a nucleic acid encoding a CAST system component is codon optimized for a cotton cell. In another aspect, a nucleic acid encoding a CAST system component is codon optimized for an alfalfa cell. In another aspect, a nucleic acid encoding a CAST system component is codon optimized for a barley cell. In another aspect, a nucleic acid encoding a CAST system component is codon optimized for a sorghum cell. In another aspect, a nucleic acid encoding a CAST system component is codon optimized for a sugarcane cell. In another aspect, a nucleic acid encoding a CAST system component is codon optimized for a canola cell. In another aspect, a nucleic acid encoding a CAST system component is codon optimized for a tomato cell. In another aspect, a nucleic acid encoding a CAST system component is codon optimized for an Arabidopsis cell. In another aspect, a nucleic acid encoding a CAST system component is codon optimized for a cucumber cell. In another aspect, a nucleic acid encoding a CAST system component is codon optimized for a potato cell. In another aspect, a nucleic acid encoding a CAST system component is codon optimized for a monocotyledonous plant cell. In another aspect, a nucleic acid encoding a CAST system component is codon optimized for a dicotyledonous plant cell.

Several embodiments relate to a ShCAST system that is optimized for expression in plant cells. The gene sequences encoding the Cas12k, tnsB, tnsC and tniQ proteins of the ShCAST system are optimized for expression in plant cells. In some embodiments, a codon optimized sequence encoding tnsB is selected from SEQ ID NO: 1, 2, 13, 14 and 15. In some embodiments, a codon optimized sequence encoding tnsC is selected from SEQ ID NO: 3, 4, 16, 17 and 18. In some embodiments, a codon optimized sequence encoding tniQ is selected from SEQ ID NO: 5, 6, 19, 20 and 21. In some embodiments, a codon optimized sequence encoding Cas12k is selected from SEQ ID NO: 7, 8, 22, 23 and 24.

In some embodiments, the gene sequences encoding the Cas12k, tnsB, tnsC and tniQ proteins of the AcCAST system are optimized for expression in plant cells. In some embodiments, a codon optimized sequence encoding tnsB is selected from SEQ ID NO: 9, 25, 26 and 27. In some embodiments, a codon optimized sequence encoding tnsC is selected from SEQ ID NO: 10, 28, 29 and 30. In some embodiments, a codon optimized sequence encoding tniQ is selected from SEQ ID NO: 11, 31, 32 and 33. In some embodiments, a codon optimized sequence encoding Cas12k is selected from SEQ ID NO: 12, 34, 35 and 36.

In some embodiments, sequences encoding the Cas12k, tnsB, tnsC and tniQ proteins of the AcCAST and ShCAST systems are operably linked to plant-specific regulatory elements. For example, for expression in soybean, a ubiquitin promoter from Medicago truncatula (MtUbq) or the 35S promoter from Dahlia mosaic virus (DaMV 35S) can be used to drive expression of CAST proteins.

In some embodiments, the protein coding regions of CAST effector gene cassettes contain a functional intron sequence, designed to reduce the impact of leaky expression of the effector cassettes in Agrobacterium tumefaciens. In plants, the inclusion of some introns in gene constructs leads to increased mRNA and protein accumulation relative to constructs lacking the intron. This effect has been termed “intron mediated enhancement” (IME) of gene expression. Introns known to stimulate expression in plants have been identified in maize genes (e.g., tubA1, Adh1, Sh1, and Ubi1), in rice genes (e.g., tpi) and in dicotyledonous plant genes like those from petunia (e.g., rbcS), potato (e.g., st-1s1) and from Arabidopsis thaliana (e.g., ubq3 and pat1). It has been shown that deletions or mutations within the splice sites of an intron reduce gene expression, indicating that splicing might be needed for IME. However, IME in dicotyledonous plants has been shown by point mutations within the splice sites of the pat1 gene from A. thaliana. Multiple uses of the same intron in one plant has been shown to exhibit disadvantages. In those cases, it is necessary to have a collection of basic control elements for the construction of appropriate recombinant DNA elements.

It can be desirable to direct a CAST system component to the nucleus of a plant cell. In such instances, one or more nuclear localization signals can be used to direct the localization of the CAST system component. As used herein, a “nuclear localization signal” refers to an amino acid sequence that “tags” a protein (e.g., a tnsB, tnsC, tniQ, or Cas12k) for import into the nucleus of a cell. In an aspect, a nucleic acid molecule provided herein encodes a nuclear localization signal. In another aspect, a nucleic acid molecule provided herein encodes two or more nuclear localization signals. In an aspect, a CAST protein provided herein comprises a nuclear localization signal. In an aspect, a nuclear localization signal is positioned on the N-terminal end of a CAST protein. In a further aspect, a nuclear localization signal is positioned on the C-terminal end of a CAST protein. In yet another aspect, a nuclear localization signal is positioned on both the N-terminal end and the C-terminal end of a CAST protein. In some embodiments, sequences encoding Nuclear localization signal peptides that are functional in plant cells are fused to the 5′ and/or 3′ end of the protein open reading frame to localize the CAST proteins to the nuclease of plant cells.

In some embodiments, sequences encoding components of the CAST system can be placed in separate expression vectors. In other embodiments, sequences encoding two or more components of the CAST system can be placed in the same expression vector. In some embodiments, sequences encoding all four proteins of the CAST system can be placed into the same expression vector. In embodiments where sequences encoding two or more CAST proteins are in the same expression vector, the genes encoding the protein components of the CAST system can be driven by diverse or similar regulatory elements. In some embodiments, fusion constructs are created among two, three or all four CAST protein coding genes, which are placed within the same open reading frame separated by flexible oligopeptide linkers. Not wishing to be bound by a particular theory, a fused configuration coordinates expression of the protein components of the CAST system, which is important if functions of transgenes are also meant to be coordinated. In some embodiments, two, three or all four CAST protein coding genes are operably linked to a single promoter and the protein coding sequences are separated by sequences encoding a self-cleaving peptide, such as the viral derived 2A sequence, resulting in precise cleavage separating the proteins (see Lee et. al., J Exp Bot. 2012 August; 63(13):4797-810; Liu et. al., Plant Biotechnol J. 2018 June; 16(6):1107-1109). In some embodiments, internal ribosome entry sites (IRES) sequences can be included in transcriptional cassettes to produce a transcript that results in the production of multiple polypeptides (see Gouiaa and Khoudi Phytochemistry. 2015 September; 117:537-546). In some embodiments, a protease recognition sequence, for example the Tobacco Etch Virus (TEV) NIa protease recognition sequence (heptapeptide cleavage recognition sequence ENLYFQS) is used together with the NIa proteinase to produce two or more polypeptides from a single transcription unit.

While not being limited by any particular scientific theory, the Cas12k protein of the CAST system forms a complex with a guide nucleic acid, which hybridizes with a complementary sequence in a target nucleic acid molecule, thereby guiding the Cas12k protein to the target nucleic acid molecule and insertion of the donor cassette at the target site. In some embodiments, the guide nucleic acid comprises: a first segment comprising a nucleotide sequence that is complementary to a sequence in a target nucleic acid and a second segment that interacts with the Cas12k protein. In some embodiments, the first segment of a guide comprising a nucleotide sequence that is complementary to a sequence in a target nucleic acid corresponds to a CRISPR RNA (crRNA or crRNA repeat). In some embodiments, the second segment of a guide comprising a nucleic acid sequence that interacts with the Cas12k protein corresponds to a trans-acting CRISPR RNA (tracrRNA). In some embodiments, the guide nucleic acid comprises two separate nucleic acid molecules (a polynucleotide that is complementary to a sequence in a target nucleic acid and a polynucleotide that interacts with a catalytically inactive CRISPR associated protein) that hybridize with one another and is referred to herein as a “double-guide” or a “two-molecule guide”. In some embodiments, the double-guide may comprise DNA, RNA or a combination of DNA and RNA. In other embodiments, the guide nucleic acid is a single polynucleotide and is referred to herein as a “single-molecule guide” or a “single-guide”. In some embodiments, the single-guide may comprise DNA, RNA or a combination of DNA and RNA. Several embodiments relate to a single guide RNA (sgRNA) comprising crRNA and tracrRNA created by using a short synthetic oligonucleotide (‘loop’) between the two. The term “guide nucleic acid” is inclusive, referring both to double-molecule guides and to single-molecule guides. Expression of guide nucleic acids can be driven by standard snRNA promoters for example promotors from U6, 7SL, U2, U5, and U3 class of small RNAs (See US20170166912A1, herein incorporated by reference.) In some embodiments, expression of a guide nucleic acid is driven by the U6i promoter. In some embodiments, expression of a guide nucleic acid is driven by a U3 promoter.

### Donor Cassettes

While not being limited by any particular scientific theory, the CAST system utilizes a donor cassette carrying a recognizable ‘transposon’ for successful transposition (see Strecker et al., Science10.1126/science.aax9181(2019). The conserved left end boundary sequence (LE) and right end boundary sequence (RE) elements provides this recognition. In a donor cassette, a nucleic acid sequence of interest (SOI) is flanked by LE and RE elements. In some embodiments, the donor cassette can comprise the coding region of a reporter gene, which, if integrated downstream of a native promoter, will provide a quick read-out of targeted transposition before further, DNA sequence-based confirmation. In soy, the spectinomycin adenylyl-transferase (aadA) or green fluorescence protein are examples of selectable marker genes and reporter genes, respectively. In some embodiments, the sequence of interest comprises one or more genes of agronomic interest.

In some embodiments, the sequence of interest comprises one or more genes conferring male sterility. Examples of genes conferring male sterility include those disclosed in U.S. Pat. Nos. 3,861,709; 3,710,511; 4,654,465; 5,625,132; and 4,727,219. The use of herbicide-inducible male sterility genes is described in U.S. Pat. No. 6,762,344. Induced male sterility in transgenic plants can increase the efficiency of hybrid seed production by eliminating the need to physically emasculate plants used as a female in a given cross.

In some embodiments, the sequence of interest comprises one or more genes conferring herbicide tolerance. Numerous herbicide resistance genes are known and may be employed with the invention. An example is a gene conferring resistance to an herbicide that inhibits the growing point or meristem, such as an imidazalinone or a sulfonylurea. Examples of genes in this category code for mutant ALS and AHAS enzyme as described, for example, by Lee et al., EMBO J., 7:1241, 1988; Gleen et al., Plant Molec. Biology, 18:1185-1187, 1992; and Miki et al., Theor. Appl. Genet., 80:449, 1990. Resistance genes for glyphosate (resistance conferred by mutant 5-enolpyruvl-3 phosphikimate synthase (EPSPS) and aroA genes, respectively) and other phosphono compounds such as glufosinate (phosphinothricin acetyl transferase (PAT) and Streptomyces hygroscopicus phosphinothricin-acetyl transferase (bar) genes) may also be used. See, for example, U.S. Pat. No. 4,940,835 to Shah, et al., which discloses the nucleotide sequence of a form of EPSPS which can confer glyphosate resistance. Examples of specific EPSPS expression cassettes conferring glyphosate resistance are provided by U.S. Pat. No. 6,040,497. Among DNA sequences encoding proteins which confer properties of tolerance to certain herbicides also includes the bar or PAT gene or the Streptomyces coelicolor gene described in WO2009/152359 which confers tolerance to glufosinate herbicides, a gene encoding glyphosate-n-acetyltransferase, or a gene encoding glyphosate oxidoreductase. Further suitable herbicide tolerance traits include at least one ALS (acetolactate synthase) inhibitor (e.g. WO2007/024782), a mutated Arabidopsis ALS/AHAS gene (e.g. U.S. Pat. No. 6,855,533), genes encoding 2,4-D-monooxygenases conferring tolerance to 2,4-D (2,4-dichlorophenoxyacetic acid) and genes encoding Dicamba monooxygenases conferring tolerance to dicamba (3,6-dichloro-2-methoxybenzoic acid).

In some embodiments, the sequence of interest comprises one or more genes conferring disease resistance. Plant defenses are often activated by specific interaction between the product of a disease resistance gene (R) in the plant and the product of a corresponding avirulence (Avr) gene in the pathogen. A resistance gene can be provided in the donor cassette to produce plants that are resistant to specific pathogen strains. See, for example Jones et al., Science, 266:7891, 1994 (cloning of the tomato Cf-9 gene for resistance to Cladosporium fulvum); Martin et al., Science, 262: 1432, 1993 (tomato Pto gene for resistance to Pseudomonas syringae pv.); and Mindrinos et al., Cell, 78(6):1089-1099, 1994 (Arabidopsis RPS2 gene for resistance to Pseudomonas syringae). A viral-invasive protein or a complex toxin derived therefrom may also be used for viral disease resistance. For example, the accumulation of viral coat proteins expressed in plant cells imparts resistance to viral infection and/or disease development effected by the virus from which the coat protein gene is derived, as well as by related viruses (see Beachy et al., Ann. Rev. Phytopathol., 28:451, 1990). Coat protein-mediated resistance can be conferred upon plants against alfalfa mosaic virus, cucumber mosaic virus, tobacco streak virus, potato virus X, potato virus Y, tobacco etch virus, tobacco rattle virus, and tobacco mosaic virus.

In some embodiments, the sequence of interest comprises one or more genes conferring insect resistance. One example of an insect resistance gene includes a gene encoding a Bacillus thuringiensis protein, a derivative thereof, or a synthetic polypeptide modeled thereon. Examples of insect resistance genes includes genes encoding Bt Cry or VIP proteins which include the Cry1A, CryIAb, CryIAc, CryIIA, CryIIIA, CryIIIB2, Cry9c Cry2Ab, Cry3Bb and CryIF proteins or toxic fragments thereof and also hybrids or combinations thereof, especially the Cry1F protein or hybrids derived from a Cry1F protein (e.g. hybrid Cry1A-Cry1F proteins or toxic fragments thereof), the Cry1A-type proteins or toxic fragments thereof, the Cry1Ac protein or hybrids derived from the Cry1Ac protein (e.g. hybrid Cry1Ab-Cry1Ac proteins) or the Cry1Ab or Bt2 protein or toxic fragments thereof, the Cry2Ae, Cry2Af or Cry2Ag proteins or toxic fragments thereof, the Cry1A.105 protein or a toxic fragment thereof, the VIP3Aa19 protein, the VIP3Aa20 protein, the VIP3A proteins produced in the COT202 or COT203 cotton events, the VIP3Aa protein or a toxic fragment thereof as described in Estruch et al. (1996), Proc Natl Acad Sci US A. 28; 93(11):5389-94, the Cry proteins as described in WO2001/47952, the insecticidal proteins from Xenorhabdus (as described in WO98/50427), Serratia (particularly from S. entomophila) or Photorhabdus species strains, such as Tc-proteins from Photorhabdus as described in WO98/08932. Also any variants or mutants of any one of these proteins differing in some amino acids (1-10, preferably 1-5) from any of the above named sequences, particularly the sequence of their toxic fragment, or which are fused to a transit peptide, such as a plastid transit peptide, or another protein or peptide, is included herein.

In some embodiments, the sequence of interest comprises one or more genes conferring quality improvements such as yield, nutritional enhancements, environmental or stress tolerances, or any desirable changes in plant physiology, growth, development, morphology or plant product(s) including starch production (U.S. Pat. Nos. 6,538,181; 6,538,179; 6,538,178; 5,750,876; 6,476,295), modified oils production (U.S. Pat. Nos. 6,444,876; 6,426,447; 6,380,462), high oil production (U.S. Pat. Nos. 6,495,739; 5,608,149; 6,483,008; 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,541,259; 5,985,605; 6,171,640), biopolymers (U.S. Pat. Nos. RE37,543; 6,228,623; 5,958,745 and U.S. Patent Publication No. US20030028917). In addition, genes of agronomic interest envisioned by this disclosure would include but are not limited to genes that confer environmental stress resistance (U.S. Pat. No. 6,072,103), pharmaceutical peptides and secretable peptides (U.S. Pat. Nos. 6,812,379; 6,774,283; 6,140,075; 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; 5,869,720) and biofuel production (U.S. Pat. No. 5,998,700). Any of these or other genetic elements, methods, and transgenes can be used with the disclosure as will be appreciated by those of skill in the art in view of this disclosure.

In some embodiments, the sequence of interest comprises a gene of agronomic interest that can affect plant characteristics or phenotypes by encoding a RNA molecule that causes the targeted modulation of gene expression of an endogenous gene, for example by antisense (see, e.g. U.S. Pat. No. 5,107,065); inhibitory RNA (“RNAi,” including modulation of gene expression by miRNA-, siRNA-, trans-acting siRNA-, and phased sRNA-mediated mechanisms, e.g., as described in published applications U.S. 2006/0200878 and U.S. 2008/0066206, and in U.S. patent application Ser. No. 11/974,469); or cosuppression-mediated mechanisms. The RNA could also be a catalytic RNA molecule (e.g., a ribozyme or a riboswitch; see, e.g., U.S. 2006/0200878) engineered to cleave a desired endogenous mRNA product. Methods are known in the art for constructing and introducing constructs into a cell in such a manner that the transcribable DNA molecule is transcribed into a molecule that is capable of causing gene suppression.

In some embodiments, the sequence of interest comprises a selectable marker. As used herein the term “selectable marker transgene” refers to any transcribable DNA molecule whose expression in a transgenic plant, tissue or cell, or lack thereof, can be screened for or scored in some way. Selectable marker genes, and their associated selection and screening techniques, for use in the practice of the invention are known in the art and include, but are not limited to, transcribable DNA molecules encoding β-glucuronidase (GUS), green fluorescent protein (GFP), proteins that confer antibiotic resistance, and proteins that confer herbicide tolerance.

### Delivering CAST Reagents for Ex Planta Assays

CAST constructs designed for ex planta experiments can be delivered into plant protoplast using any of these standard methods known in the art. Microinjection, electroporation, vacuum infiltration, pressure, sonication, silicon carbide fiber agitation, PEG-mediated transformation, etc., are some of the methods known in the art.

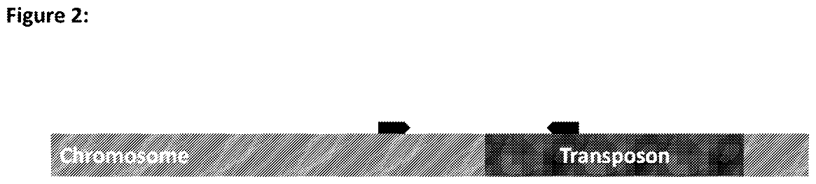

In one embodiment, CAST constructs designed for ex planta experiments in soy protoplasts may be delivered via polyethylene glycol (PEG)-mediated transformation. Soy protoplasts are generated from cotyledon using known protocols in the art and polyethylene glycol (PEG)-mediated transformation is used for co-delivery of expression constructs encoding the CAST system components in set molar ratios. Following a two-day incubation, total genomic DNA is isolated and molecular assays such as ‘flank PCR’ between a primer specific to the transposon cassette and another primer located proximal to the chromosomal target site is used to detect and quantify targeted transpositions. Sequencing of the resulting amplicons provides the evidence for targeted transposition (See FIG. 2).

Delivery of CAST System Components into Plants

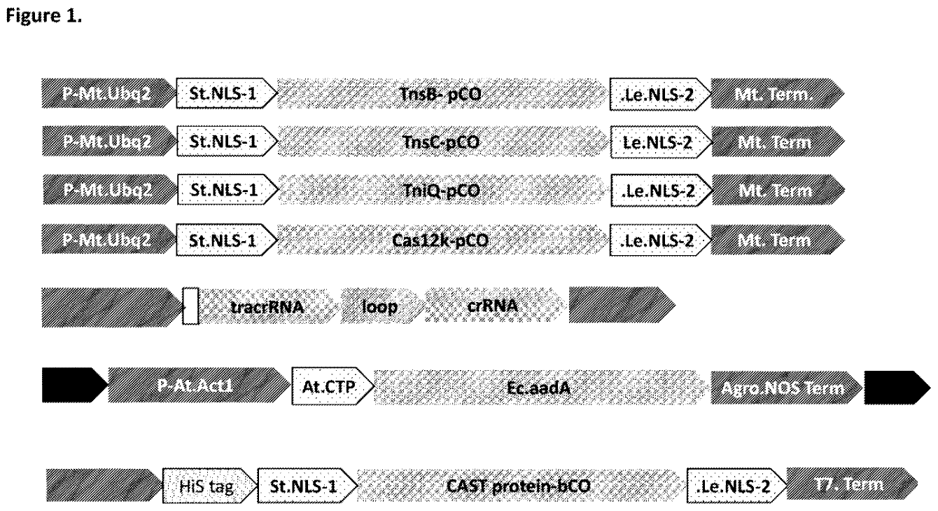

Several embodiments relate to delivery of the four CAST system proteins as mRNA or protein and the guide nucleic acid directly to plant cells. Not wishing to be bound by any particular theory, direct delivery of RNA or protein to plant cells could provide rapid, concerted activity of the CAST system soon after delivery, thus avoiding dependency on synchronized gene expression in vivo. In some embodiments, components of the CAST system can be delivered as ribonucleoprotein (RNP) complexes. This could also allow adjustment of molar ratios of components prior to transformation to improve efficacy. Methods of delivering CRISPR RNP complexes is described in PCT/US2019/033976 and incorporated by reference herein, in its entirety. For RNP based delivery, the protein-coding elements of CAST are codon-optimized for optimal expression in bacteria, for example Escherichia coli. In one embodiment, the sequences are operably linked to prokaryotic TAC promoter followed by 5′ 7xHis tag for Ni-column purification and introduced into a suitable bacterial expression vector (See FIG. 1D). In some embodiments, the protein components of the CAST system are engineered to remove cysteines. Cysteine residues in a protein are able to form disulfide bridges providing a strong reversible attachment between cysteines. To control and direct the attachment of the protein components of the CAST system in a targeted manner the native cysteines are removed to control the formation of these bridges. Not wishing to be bound by a particular theory, removal of the cysteines from the protein backbone would enable targeted insertion of new cysteine residues to control the placement of these reversible connections by a disulfide linkage. This could be between protein components of the CAST system or to a particle such as a gold particle for biolistic delivery. A tag comprising several residues of cysteine could be added to the protein components of the CAST system that would allow it to specifically attach to metal beads (specifically gold) in a uniform way.

Numerous methods for transforming chromosomes or plastids in a plant cell with a recombinant DNA molecule are known in the art, which can be used according to methods of the present application to produce a plant cell and plant comprising components of the CAST system.

In planta, particle bombardment or biolistic delivery can be used for delivering multi-component systems, such as CAST. Particle bombardment is suitable to transform plants with DNA, RNA, protein, or any combinations thereof. Methods of transforming plants via biolistic delivery of RNP complexes is described in PCT/US2019/033976 and incorporated by reference herein, in its entirety. Methods of transforming plants using biolistic delivery of DNA is described in PCT/US2019/033984 and incorporated by reference herein, in its entirety.

In planta, Agrobacterium mediated transformation is a suitable method of choice for delivering multi-component systems, such as CAST, on one or more expression cassettes provided on one or more T-DNAs. Agrobacterium mediated transformation is widely applied to monocot and dicot species. The expression cassettes comprising one or more components of the CAST system may be provided, in one embodiment, as double tumor-inducing (Ti) plasmid border constructs that have the right border (RB or AGRtu.RB) and left border (LB or AGRtu.LB) regions of the Ti plasmid isolated from Agrobacterium tumefaciens comprising a T-DNA that, along with transfer molecules provided by the A. tumefaciens cells, permit the integration of the T-DNA into the genome of a plant cell (see, e.g., U.S. Pat. No. 6,603,061). The constructs may also contain the plasmid backbone DNA segments that provide replication function and antibiotic selection in bacterial cells, e.g., an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. In some embodiments, one or more expression cassettes encoding one or more CAST system components are provided in a T-DNA binary vector that has a low copy origin of replication, such as the OriRi vector backbone. For plant transformation, the host bacterial strain is often A. tumefaciens ABI, C58, or LBA4404, however other strains known to those skilled in the art of plant transformation can function in the invention. In some embodiments, an Agrobacterium tumefaciens strain that lacks certain DNA recombination functions, such as RecA, is utilized to deliver expression vectors encoding CAST system components to plant cells.

In some embodiments, the expression cassettes encoding components of the CAST system as described herein are provided on a single T-DNA. In some embodiments, the expression cassettes encoding components of the CAST system as described herein are provided on multiple separate T-DNAs and delivered to plant cells in a single transformation process, or in separate sequential transformation processes. In some embodiments, sequences encoding the protein components of the CAST system are provided to a plant cell on a separate T-DNA vector than sequences encoding the guide nucleic acid component(s) of the CAST system. In some embodiments, sequences encoding the protein components of the CAST system are provided to a plant cell on a separate T-DNA vector than sequences encoding the guide nucleic acid component(s) of the CAST system and the donor cassette. In some embodiments, sequences encoding the protein components of the CAST system and sequences encoding the guide nucleic acid component(s) of the CAST system are provided to a plant cell on a separate T-DNA vector than and the donor cassette. In some embodiments, sequences encoding the protein components of the CAST system and sequences encoding the guide nucleic acid component(s) of the CAST system are provided to a plant cell on a separate T-DNA vector than and the donor cassette. In some embodiments, sequences encoding the protein components of the CAST system and the donor cassette are provided to a plant cell by Agrobacterium-based transformation and sequences encoding the guide nucleic acid component(s) of the CAST system are provided by particle bombardment. In some embodiments, the donor cassette is provided to a plant cell by Agrobacterium-based transformation and the protein components of the CAST system and sequences encoding the guide nucleic acid component(s) of the CAST system are provided by particle bombardment.

In some embodiments, the genetic elements of the CAST system are delivered into separate plants such that no single primary plant contains all of the elements necessary to activate transposition. Transposition is activated by combining all of the necessary elements into a progeny plants created by crossing plants that contain some of the elements. In some embodiments, a plant that contains functional genes for all of the effector proteins (TnsB, TnsC, TniQ and Cas12k) are crossed to plants that contain the ‘donor’ cassette carrying a recognizable ‘transposon’ and a guide nucleic acid expression cassette, whereby targeted transposition of the donor cassette into a specific site occurs in progeny from such a cross. In some embodiments, a plant that contains functional genes for all of the effector proteins (TnsB, TnsC, TniQ and Cas12k) and a ‘donor’ cassette carrying a recognizable ‘transposon’) are crossed to plants that contain a guide nucleic acid expression cassette, whereby targeted transposition of the donor cassette into a specific site occurs in progeny from such a cross. In some embodiments, a plant that contains functional genes for all of the effector proteins (TnsB, TnsC, TniQ and Cas12k) and a guide nucleic acid expression cassette are crossed to plants that contain the ‘donor’ cassette carrying a recognizable ‘transposon’, whereby targeted transposition of the donor cassette into a specific site occurs in progeny from such a cross. This strategy of combining elements through plant crosses applies to methods that utilize particle bombardment as well as methods that utilize Agrobacterium tumefaciens to create transgenic plants. For example, particles comprising all of the effector proteins (TnsB, TnsC, TniQ and Cas12k) and a guide nucleic acid can be bombarded into plants that contain a ‘donor’ cassette carrying a recognizable ‘transposon’.

In some embodiments, tight developmental or inducible control of the expression of tnsB, tnsC, tniQ, Cas12k and/or the guide nucleic acid is utilized to prevent premature transposition. In some embodiments, an ethanol inducible promoter is used to drive expression of components of the CAST system. Another option to prevent premature transposition is to separate the protein (tnsB, tnsC, tniQ, and Cas12k) and guide nucleic acid components into different vectors and transforming them into different plants, which are then crossed to activate targeted transposition in the progeny. A donor cassette may be transformed into either parent plant, either on the same T-DNA as the transposase and/or chimeric targeting gRNA or on a separate T-DNA.

In some embodiments, premature transposition is prevented by providing a guide nucleic acid that does not recognize a target site in the transformation germplasm. When a plant containing the CAST components is then crossed to a plant comprising a target site, targeted transposition occurs.

Targeted transpositions can be detected by ‘flank PCR’ in both protoplasts and plants. However, in case of large-scale stable, in planta transformations yielding hundreds, if not thousands of transformants, higher-throughput detection methods are desirable. Chromosome phasing is a high-throughput, TaqMan-based method designed for detecting physical linkage of markers using digital PCR (See Regan, J. and G. Karlin-Neumann, 2018, Methods Mol Biol 1768: 489-512.) With an assay designed to the target region and another one on the transposon of interest, chromosome phasing can readily identify targeted transposition events in a high throughput manner. It could also detect off-target transpositions side-by-side with the on-target ones without the need for additional experimentation.

### Use of Genome Editing in Molecular Breeding and Trait Integration

In some embodiments, genome knowledge is utilized for targeted transposition. In one embodiment, a guide nucleic acid can be used to target Cas12k to at least one region of a genome to disrupt that region of the genome in a plant cell. A modification based on a donor DNA template can then be introduced within that genomic region. A plant regenerated from a modified plant cell comprises a modified genome and may exhibit a modified phenotype or other property depending on the genetic region that has been altered. Previously characterized mutant alleles or transgenes can be targeted for modification using the CAST system, enabling the creation of improved mutants or transgenic lines.

In some embodiments, a gene targeted for deletion or disruption by targeted transposition may be a transgene that was previously introduced into the target plant or cell. This has the advantage of allowing a different transgene to be introduced or allowing disruption and/or removal of sequence encoding a selectable marker. In yet another embodiment, a gene targeted for modification via genome editing is at least one transgene that was introduced on the same vector or expression cassette as one or more other transgenes of interest and resides at the same locus as another transgene. It is understood by those skilled in the art that this type of genome modification may result in deletion or insertion of additional sequences at the targeted locus. In some embodiments, a specific transgene may be disrupted while leaving the remaining transgene(s) intact. This avoids having to create a new transgenic line containing the desired transgenes without the undesired transgene.

In another aspect, the present disclosure includes methods for inserting a donor DNA sequence of interest into a specific site of a plant genome, wherein the DNA sequence of interest is from the genome of the plant or is heterologous with respect to the plant. This disclosure allows one to select for cells in which a particular region of the genome has been modified for insertion of one or more expression cassettes by targeted transposition. A targeted region of the genome may thus display linkage of at least one transgene to a haplotype of interest associated with at least one phenotypic trait and may also result in the development of a linkage block to facilitate transgene stacking and transgenic trait integration, and/or development of a linkage block while also allowing for conventional trait integration.

Directed chromosome rearrangement allows multiple nucleic acids of interest (e.g., a trait stack or multi-plexing) to be added to the genome of a plant in either the same site or different sites. Sites for targeted transposition can be selected based on knowledge of the underlying breeding value, transgene performance in that location, underlying recombination rate in that location, existing transgenes that are linked to the site for targeted transposition, or other factors. Once the stacked plant is assembled, it can be used as a trait donor for crosses to germplasm being advanced in a breeding program or be directly advanced in the breeding program.

The present disclosure includes methods for inserting at least one nucleic acid of interest into at least one site in a plant genome, wherein the nucleic acid of interest is from the genome of a plant, such as a QTL or allele, or is transgenic in origin. A targeted region of the genome may thus display linkage of at least one transgene to a haplotype of interest associated with at least one phenotypic trait (as described in U.S. Patent Application Publication No. 2006/0282911), to facilitate transgene stacking, transgenic trait integration, QTL or haplotype stacking, and conventional trait integration.

In some embodiments, multiple unique guide molecules can be used to modify multiple alleles at specific loci within one linkage block contained on one chromosome by making use of knowledge of genomic sequence information and the ability to design custom guide molecules. A guide molecule that is specific for, or can be directed to, a genomic target site that is upstream of the locus containing the non-target allele is designed or engineered as necessary. A second guide molecule that is specific for, or can be directed to, a genomic target site that is downstream of the target locus containing the non-target allele is also designed or engineered. The guide molecules may be designed such that they complement genomic regions where there is no homology to the non-target locus containing the target allele. Both guide molecules may be introduced into a cell using one of the methods described herein.

Several embodiments relate to targeted transposition utilizing the CAST system to create blocks of genetically linked loci (a megalocus) that can be transmitted as a single genetic unit through a trait introgression process to other plants, varieties or species. In some embodiments, a donor cassette is inserted by targeted transposition into a locus that is genetically linked but physically separate from an existing transgene insertion site, or a set of transgene insertion sites/events. In some embodiments, a megalocus is formed by inserting donor cassettes from different CAST system into loci that are genetically linked but physically separate. In some embodiments, a donor cassette comprising a ShLE and a ShRE is inserted by targeted transposition into a locus that is genetically linked but physically separate from an existing donor cassette comprising an AcLE and an AcRE. In some embodiments, a donor cassette comprising an AcLE and an AcRE is inserted by targeted transposition into a locus that is genetically linked but physically separate from an existing donor cassette comprising a ShLE and a ShRE. In one embodiment, targeted transposition of at least one transgene that produces a desirable trait in a plant is followed by recombination linking a second transgene to form a megalocus. Such an approach of targeted transformation followed by recombination to link desired transgenes possesses advantages of both vector stacks and breeding stacks without many of the limitations. For example, in one embodiment, individual transgenes may be introduced by targeted transposition one at a time and combined at a later date. In some embodiments, targeted transposition of at least one transgene occurs at a target site that is genetically linked a second transgene to form a megalocus. In some embodiments, transposition sites may be physically separated from a locus of interest by a distance of between about 0.1 cM to about 20 cM, including 0.1, 0.2, 0.3, 0.4, 0.5, 0.6, 0.7, 0.8, 0.9, 1, 1.2, 1.3, 1.4, 1.5, 1.6, 1.7, 1.8, 1.9, 2, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 11, 12, 13, 14, 15, 16, 17, 18, 19, and 20 cM. In a further embodiment, the transposition site of individual donor cassettes may not be genetically linked, or may not be closely linked, such as at least about 10, 20, 30, 40 or more cM apart. Once donor cassettes are combined in cis on the same chromosome, they could be induced to be genetically linked by chromosome rearrangement of the intervening sequences, thus allowing numerous independent transgenes to be easily introgressed into different germplasm. In a further embodiment, two plant lines, each containing different transgenes that have been combined to form a megalocus at a linked site in trans, can be crossed together to create one large megalocus in cis, containing all of the transgenes.

Linking transgenic traits together as a genetic linkage block may be desirable due to the ability to reduce the number of randomly segregating transgenic loci in the trait integration process. Stacking of transgenes that are genetically linked may also reduce the number of progeny to be screened to find stacked transgenes during the trait integration process. Additionally, combining targeted transposition and utilizing the endogenous meiotic recombination machinery to link transgenes provides extra flexibility in product concepts that speeds up product delivery timelines.

A further embodiment of the invention is the combination of targeted transposition with technology to modify meiotic recombination machinery wherein such technology includes transgenic modification of gene expression or chemical treatments to modulate recombination. In some embodiments, targeted transposition of a donor cassette is combined with cleavage by a site-specific genome modification enzyme, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system) to modify recombination rates. Genetically linking traits by recombination effectively reduces trait loci for trait introgression while still providing flexibility. For instance, by employing methods of the present invention, several transgenes conferring the same or different traits may be tested at the same loci, rather than vector stacking the traits, allowing testing of several combinations of traits and versions of traits simultaneously before deciding on a commercial product. With vector stacking, it is necessary to make decisions regarding commercial product concepts several years in advance, which reduces flexibility. In accordance with some embodiments of the present invention, a next-generation trait may be tested at the same locus or nearby locus as a previous trait, which may then replace the previous trait by recombining out the previous trait and recombining in the next-generation trait. This invention also anticipates inclusion of target recognition sites within donor cassettes to enable insertion and deletion of transgenes and transgenic elements within at least one donor cassette.

Several embodiments relate to the targeted transposition of a donor cassette into a target site that is about 0.1 cM to about 20 cM, including 0.1, 0.2, 0.3, 0.4, 0.5, 0.6, 0.7, 0.8, 0.9, 1, 1.5, 2, 2.5, 3, 5, 10, 15, and 20 cM, from an identified quality trait locus (QTL). In some embodiments, a donor cassette is transposed into a target site that is about 0.1, 0.2, 0.3, 0.4, 0.5, 0.6, 0.7, 0.8, 0.9, 1, 1.2, 1.3, 1.4, 1.5, 1.6, 1.7, 1.8, 1.9, 2, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 cM from an identified QTL.

Several embodiments relate to the targeted transposition of a donor cassette into a target site that is about 0.1 cM to about 20 cM, including 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 and 20 cM, from a transgenic event. In some embodiments, the CAST system is utilized to provide targeted transposition of a donor cassette containing one or more transgenes into a locus that is 0.1 cM to about 20 cM, including 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 and 20 cM, from a transgenic event selected from Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946); Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control—herbicide tolerance, deposited as PTA-6233, described in WO2005/103266 or US-A 2005-216969); Event 3006-210-23 (cotton, insect control—herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control—herbicide tolerance, deposited as ATCC PTA-11508, described in WO 11/075593); Event 43A47 (corn, insect control—herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMB No. 41603, described in WO2010/080829); Event BLR1 (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.1 (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control—herbicide tolerance, deposited as ATCC PTA 11384, described in US-A 2006-070139); Event DAS-59132 (corn, insect control—herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-I (brinjal, insect control, not deposited, described in WO 07/091277); Event Fil 17 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO98/044140); Event GHB119 (cotton, insect control—herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-1 (sugar beet, herbicide tolerance, deposited as NCIMB 41158 or NCIMB 41159, described in US-A 2004-172669 or WO 2004/074492); Event JOPLIN′ (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/026345); Event LLRice62 (rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait—herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control—herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MS1 1 (oilseed rape, pollination control—herbicide tolerance, deposited as ATCC PTA-850 or PTA-2485, described in WO2001/031042); Event MS8 (oilseed rape, pollination control—herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control—herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control—herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control—herbicide tolerance, not deposited, described in US-A 2005-039226 or WO2004/099447); Event VIP1034 (corn, insect control—herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in WO2011/084621), event EE-GM3/FG72 (soybean, herbicide tolerance, ATCC Accession N° PTA-11041) optionally stacked with event EE-GM1/LL27 or event EE-GM2/LL55 (WO2011/063413A2), event DAS-68416-4 (soybean, herbicide tolerance, ATCC Accession No PTA-10442, WO2011/066360A1), event DAS-68416-4 (soybean, herbicide tolerance, ATCC Accession No PTA-10442, WO2011/066384A1), event DP-040416-8 (corn, insect control, ATCC Accession No PTA-11508, WO2011/075593A1), event DP-043A47-3 (corn, insect control, ATCC Accession No PTA-11509, WO2011/075595A1), event DP-004114-3 (corn, insect control, ATCC Accession No PTA-11506, WO2011/084621A1), event DP-032316-8 (corn, insect control, ATCC Accession No PTA-11507, WO2011/084632A1), event MON-88302-9 (oilseed rape, herbicide tolerance, ATCC Accession No PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession No. PTA-10296, WO2012/051199A2), event DAS-44406-6 (soybean, stacked herbicide tolerance, ATCC Accession No. PTA-11336, WO2012/075426A1), event DAS-14536-7 (soybean, stacked herbicide tolerance, ATCC Accession No. PTA-11335, WO2012/075429A1), event SYN-000H2-5 (soybean, herbicide tolerance, ATCC Accession No. PTA-11226, WO2012/082548A2), event DP-061061-7 (oilseed rape, herbicide tolerance, no deposit No available, WO2012071039A1), event DP-073496-4 (oilseed rape, herbicide tolerance, no deposit No available, US2012131692), event 8264.44.06.1 (soybean, stacked herbicide tolerance, Accession No PTA-11336, WO2012075426A2), event 8291.45.36.2 (soybean, stacked herbicide tolerance, Accession No. PTA-11335, WO2012075429A2), event SYHT0H2 (soybean, ATCC Accession No. PTA-11226, WO2012/082548A2), event MON88701 (cotton, ATCC Accession No PTA-11754, WO2012/134808A1), event KK179-2 (alfalfa, ATCC Accession No PTA-11833, WO2013/003558A1), event pDAB8264.42.32.1 (soybean, stacked herbicide tolerance, ATCC Accession No PTA-11993, WO2013/010094A1), event MZDT09Y (corn, ATCC Accession No PTA-13025, WO2013/012775A1).

### Haploid Induction Crosses

Trait integration is a bottleneck in elite breeding programs. Transgenes with desired traits are backcrossed many times from a donor line to the elite or recurrent parent using marker based selection. A rapid and efficient way to selectively move a transgene from a donor to a recipient germplasm in a single cross without any linkage drag would have immense value to such a breeding pipeline. As described below, expressing CAST system components in a haploid inducer plant followed by crossing and selection is one way to achieve rapid trait integration and recovery of the recurrent parent in a single cross.

Several embodiments relate to a method of selectively activating the CAST system to facilitate the targeted transposition into a non-inducer genome by selectively activating the transcription of one or more CAST system components. In some embodiments, a haploid inducer line, such as INA133 or a transformable derivative of INA133/ELMYS5, comprises in its genome transgenes encoding one or more CAST system components. In some embodiments, the haploid inducer line comprises sequences encoding the protein components of the CAST system. In some embodiments, the haploid inducer line comprises sequences encoding the protein components of the CAST system and a guide nucleic acid that does not recognize a target site in the haploid inducer line. In some embodiments, the haploid inducer line comprises a guide nucleic acid that is complementary to a target site in an elite line but not the haploid inducer line. In some embodiments, the haploid inducer line comprises expression cassettes comprising sequences encoding CAST system operably linked to an inducible promoter, such as an ethanol inducible promoter. In some embodiments, the haploid inducer line comprises expression cassettes comprising an inducible promoter operably linked to a nucleic acid sequence encoding a guide nucleic acid. In some embodiments, the haploid inducer line comprises expression cassettes comprising an inducible promoter operably linked to a nucleic acid sequence encoding one or more of tnsB, tnsC, tniQ, Cas12k. In some embodiments, the haploid inducer line comprises an expression cassette comprising an inducible promoter operably linked to a nucleic acid sequence encoding one or more of tnsB, tnsC, tniQ, Cas12k, where the protein coding sequences are separated by 2A self-cleaving peptides or internal ribosome entry sites to facilitate coordinated cleavage of the proteins or coordinated expression of each gene. In some embodiments, the haploid inducer line comprises an expression cassette comprising an inducible promoter operably linked to a nucleic acid sequence encoding one component of the CAST system and one or more expression cassettes comprising a constitutive promoter operably linked to one or more sequences encoding the other CAST system components. In some embodiments, expression of the inducible promoter is induced by exposing a plant to the inducing agent upon making the haploid induction cross. In some embodiments, expression of the inducible promoter is induced by exposing the haploid inducer plant to the inducing agent prior to crossing. In some embodiments, expression of the inducible promoter is induced by exposing the progeny of a cross between a haploid inducer parent and the recipient parent to the inducing agent.

In several embodiments, a developmental specific promoter, such as the BABYBOOM gene promoter, is used to drive zygotic gene expression from the male parent of one or more of the guide nucleic acid, or the tnsB, tnsC, tniQ, Cas12k components of the CAST system. In some embodiments, a developmental specific promoter is operably linked to a nucleic acid sequence encoding the tnsB, tnsC, tniQ, Cas12k components of the CAST system, where the protein coding sequences are separated by 2A self-cleaving peptides or IRES sites to facilitate coordinated cleavage of the proteins or coordinated expression of each gene (Khanday et al., 2019, Nature, Jan 565(7737): 91-95). In some embodiments, a developmental specific promoter is operably linked to sequences encoding at least one CAST system components and a constitutive promoter is operably linked to sequences encoding one or more other CAST system components. In some embodiments, transgenic plants are maintained as females to avoid precocious expression of the CAST system and transposition prior to exposure to the genome of interest (say, the genome encountered after a haploid induction cross). Upon making the haploid induction cross, the CAST transgenic plant is used as the male and upon zygote formation the BABYBOOM promoter is activated and thus the entire CAST system is now active and capable of facilitated the RNA-guided DNA transposition to the non-inducer genome.

In some embodiments, one or more expression vectors encoding CAST system components as described herein is transformed into a haploid inducer plant. In some embodiments, the guide nucleic acid is designed to avoid any match in the haploid inducer genome but retains a match to any non-inducer genome, such that targeted transposition does not occur in the haploid inducer plant, but is activated upon crossing the haploid inducer line to a recipient germplasm.

In some embodiments, one or more expression vectors encoding CAST system components as described herein is transformed into an inducer plant containing a supernumerary chromosome, such as a B chromosome. Events are selected that insert onto the supernumerary chromosome. A haploid induction cross is made with this event on the supernumerary chromosome and haploid offspring are selected such that they retain the supernumerary chromosome but no other chromosomes from the inducer parent. The haploid offspring are then selected for those that have transpositions into the target site containing the donor transgene. In one embodiment, an ethanol inducible promoter is used to trigger transposition after recovering haploid plants containing B chromosomes carrying the donor and CAST transgene.

In some embodiments, one or more expression vectors encoding CAST system components as described herein is transformed into a corn plant. Events are selected and then crossed onto wheat plants to produce haploids. Haploids are then screened for donor transgene transposition. In some embodiments, precocious expression of the chimeric gRNA is prevented by utilizing a wheat inducible promoter (a promoter that is present in corn but only activated upon exposure to a wheat cell), or the BABYBOOM promoter or some other early zygotic promoter that is parent-genome specific and activated upon fertilization (Khanday et al., 2019, Nature, Jan 565(7737): 91-95; Anderson et al., Developmental Cell, 43,349-358 e344).

In another embodiment, viruses or viral replicons are engineered to express all or parts of the CAST system and/or harbor a donor transgene. Upon infection of one or multiple viruses or replicons comprising the CAST system and donor transgene, transposition occurs. This might be done in combination with haploid induction where the virus or replicon is topically applied before during or after fertilization with the haploid inducer.

In any of the embodiments above, chromosome doubling methods can be applied to make doubled haploids containing the transposition.

In any of the embodiments above, any crossing-based method of haploid induction could be applied (CENH3, ig1, matrilineal, DMP, wide cross, supplemental radiation, phospholipid or derivative applications).

Targeted transpositions can be properly detected by the above-mentioned ‘flank PCR’ assay in both protoplasts and plants. However, in case of large-scale stable, in planta transformations yielding hundreds, if not thousands of transformants, higher-throughput detection methods are more desirable. Chromosome phasing is a high-throughput, TaqMan-based method designed for detecting physical linkage of markers using digital PCR (dPCR). With an assay designed next to the target region and another one on the transposon of interest, chromosome phasing can readily identify targeted transposition events in a HTP manner.

### Inactivation of the CAST System Following Targeted Transposition

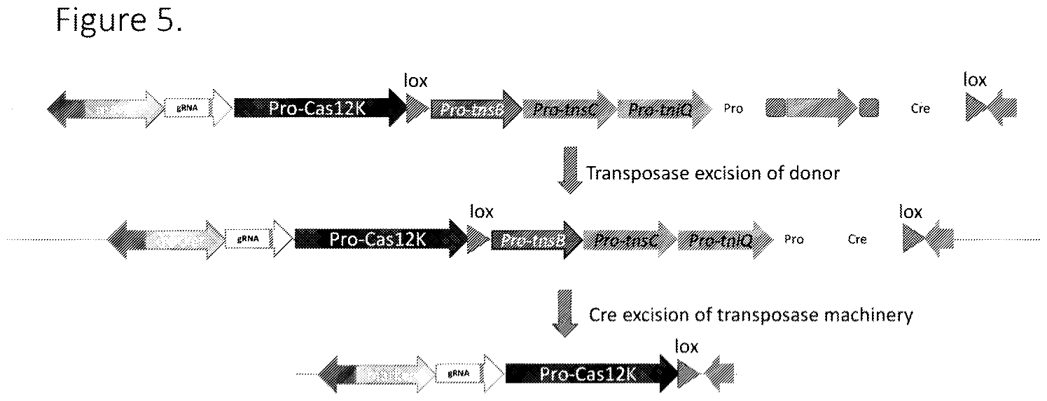

In some embodiments it may be desirable to inactivate the CAST system following targeted transposition of the donor cassette. In some embodiments, a donor cassette disrupts an expression cassette encoding site-specific recombinase, such that excision of the donor cassette results in expression of the recombinase which excises one or more components of the CAST system. In some embodiments, the donor cassette is provided between a plant expressible promoter and a sequence encoding the site-specific recombinase such that excision of the donor cassette operably links the promoter to the sequence encoding the site-specific recombinase. In some embodiments, expression of the site-specific recombinase excises the expression cassette encoding the site-specific recombinase. In some embodiments, recombinase recognition sequences are positioned such that expression of the corresponding site-specific recombinase excises one or more expression cassettes encoding one or more of tnsB, tnsC, tniQ, Cas12k and the guide nucleic acid. See e.g., FIG. 5.

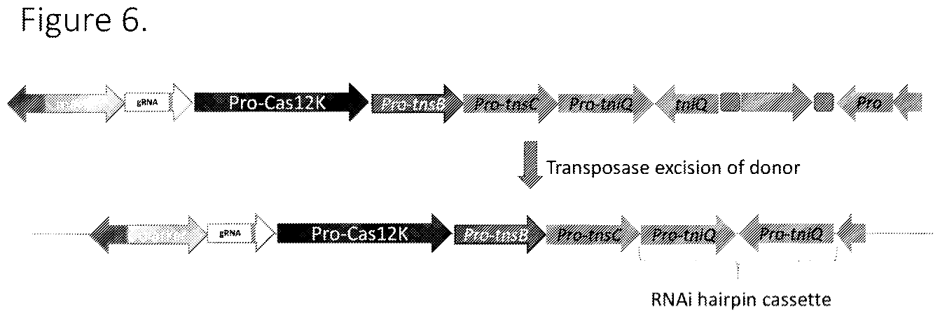

In some embodiments, RNA interference (RNAi) is utilized to suppress activity of the CAST system following targeted transposition of the donor cassette. In some embodiments, a donor cassette disrupts an expression cassette encoding a dsRNA hairpin, such that excision of the donor cassette results in expression of an antisense RNA which is complementary to tnsB, tnsC, tniQ, or Cas12k. In some embodiments, the donor cassette is provided between a plant expressible promoter and an antisense sequence that is complementary to at least 21 contiguous nucleotides of a sequence encoding tnsB, tnsC, tniQ, or Cas12k such that excision of the donor cassette operably links the promoter to the antisense sequence. See e.g., FIG. 6.

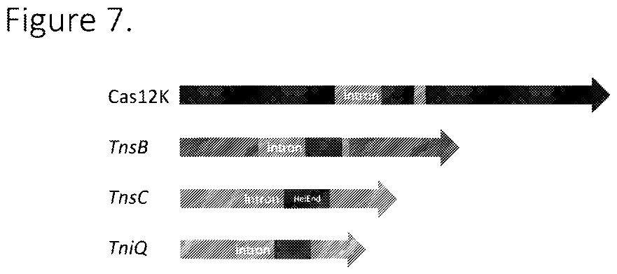

Intergenic transposons can trigger gene silencing by RNA-directed DNA methylation (RdDM). Often, silencing is delayed, thus allowing initial gene expression. In some embodiments, activity of the CAST system may be suppressed by incorporating short conserved motifs or entire non-autonomous elements of transposons into the introns or UTRs of CAST genes can silence them following an initial activity that will allow SDI. These elements include, but not restricted to long terminal repeats (LTRs) of retrotransposons, or some of their conserved motifs, such as primer binding sites (PBS), short interspersed nuclear elements (SINEs), conserved terminal repeats of Helitrons (HelEnds), and inverted terminal repeats (ITR) of DNA transposons. See e.g., FIG. 7.

### Definitions

As used herein, terms in the singular and the singular forms “a,” “an,” and “the,” for example, include plural referents unless the content clearly dictates otherwise.

“Centimorgan” or “cM” refers distance between chromosome positions for which the expected average number of intervening chromosomal crossovers in a single generation is 0.01.

“Construct” or “DNA construct” as used herein refers to a polynucleotide sequence comprising at least a first polynucleotide sequence operably linked to a second polynucleotide sequence.

“Donor cassette” or “transposon cassette” as used herein refers to a polynucleotide comprising a sequence of interest flanked by a left end boundary sequence (LE) and a right end boundary sequence (RE). In some embodiments, the sequence of interest comprises one or more expression cassettes.

“Expression cassette” as used herein refers to a polynucleotide sequence comprising at least a first polynucleotide sequence capable of initiating transcription of an operably linked second polynucleotide sequence and optionally a transcription termination sequence operably linked to the second polynucleotide sequence.

“Genomic target site” or “target site” as used herein refers to a region located in a host genome selected for targeted integration of a donor cassette.

As used herein, the term “intron” refers to a DNA molecule that may be isolated or identified from a gene and may be defined generally as a region spliced out during messenger RNA (mRNA) processing prior to translation. Alternately, an intron may be a synthetically produced or manipulated DNA element. An intron may contain enhancer elements that effect the transcription of operably linked genes, such as genes encoding tnsB, tnsC, tniQ, and Cas12k. An intron may be used as a regulatory element for modulating expression of an operably linked to a gene encoding tnsB, tnsC, tniQ, or Cas12k. A construct may comprise an intron, and the intron may or may not be heterologous with respect to the gene encoding tnsB, tnsC, tniQ, or Cas12k molecule. Examples of introns in the art include the rice actin intron and the corn HSP70 intron.

As used herein, the term “megalocus” refers to a block of at least two genetically linked loci that are normally inherited as a single unit. In some embodiments, at least one locus is a transgene. A megalocus may provide to a plant one or more desired traits, which may include, but are not limited to, enhanced growth, drought tolerance, salt tolerance, herbicide tolerance, insect resistance, pest resistance, disease resistance, and the like. In specific embodiments, a megalocus comprises at least about 2, 3, 4, 5, 6, 7, 8, 9, 10, 11, 13 or 15 transgenic loci that are physically separated but genetically linked such that they can are inherited as a single unit. In specific embodiments, a megalocus comprises at least one native trait locus and at least one transgenic locus that are physically separated but genetically linked such that they can are inherited as a single unit. Each locus in the megalocus can be 0.1, 0.2, 0.3, 0.4, 0.5, 0.6, 0.7, 0.8, 0.9, 1, 1.2, 1.3, 1.4, 1.5, 1.6, 1.7, 1.8, 1.9, 2, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 cM apart from one another.

As used herein, the term “operably linked” refers to a first DNA molecule joined to a second DNA molecule, wherein the first and second DNA molecules are so arranged that the first DNA molecule affects the function of the second DNA molecule. The two DNA molecules may or may not be part of a single contiguous DNA molecule and may or may not be adjacent. For example, a promoter is operably linked to a transcribable DNA molecule if the promoter modulates transcription of the transcribable DNA molecule of interest in a cell. A leader, for example, is operably linked to DNA sequence when it is capable of affecting the transcription or translation of the DNA sequence.

“PAM site” or “PAM sequence” as used herein refers to the protospacer adjacent motif (or PAM), which is a short DNA sequence (usually 2-6 base pairs in length) that is adjacent to the DNA region targeted for cleavage by a CRISPR associate protein/guide nucleic acid system, such as CRISPR-Cas9 or CRISPR-Cpf1. Some CRISPR associated proteins (e.g., Type I and Type II) require a PAM site in order to bind a target nucleic acid.

“Percent identity” or “% identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence.

“Plant” refers to a whole plant any part thereof, or a cell or tissue culture derived from a plant, comprising any of: whole plants, plant components, or organs (e.g., leaves, stems, roots, etc.), plant tissues, seeds, plant cells, and/or progeny of the same. A plant cell is a biological cell of a plant, taken from a plant or derived through culture from a cell taken from a plant.

“Promoter” as used herein refers to a nucleic acid sequence located upstream or 5′ to a translational start codon of an open reading frame (or protein-coding region) of a gene and that is involved in recognition and binding of RNA polymerase I, II, or III and other proteins (trans-acting transcription factors) to initiate transcription. A “plant promoter” is a native or non-native promoter that is functional in plant cells. Constitutive promoters are functional in most or all tissues of a plant throughout plant development. Tissue-, organ- or cell-specific promoters are expressed only or predominantly in a particular tissue, organ, or cell type, respectively. Rather than being expressed “specifically” in a given tissue, plant part, or cell type, a promoter may display “enhanced” expression, a higher level of expression, in one cell type, tissue, or plant part of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals.

“Recombinant” in reference to a nucleic acid or polypeptide indicates that the material (for example, a recombinant nucleic acid, gene, polynucleotide, polypeptide, etc.) has been altered by human intervention. The term recombinant can also refer to an organism that harbors recombinant material, for example, a plant that comprises a recombinant nucleic acid is considered a recombinant plant.

As used herein, the term “sequence identity” refers to the extent to which two optimally aligned polynucleotide sequences or two optimally aligned polypeptide sequences are identical. An optimal sequence alignment is created by manually aligning two sequences, e.g., a reference sequence and another sequence, to maximize the number of nucleotide matches in the sequence alignment with appropriate internal nucleotide insertions, deletions, or gaps.

As used herein, the term “percent sequence identity” or “percent identity” or “% identity” is the identity fraction multiplied by 100. The “identity fraction” for a sequence optimally aligned with a reference sequence is the number of nucleotide matches in the optimal alignment, divided by the total number of nucleotides in the reference sequence, e.g., the total number of nucleotides in the full length of the entire reference sequence. Thus, one embodiment of the invention provides a DNA molecule comprising a sequence that, when optimally aligned to a reference sequence, provided herein as SEQ ID NOs:4-13, 16-19 and 24 has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to the reference sequence.

As used herein, a “T-DNA” molecule or transfer DNA is the transferred DNA of the tumor-inducing (Ti) plasmid of some species of bacteria such as Agrobacterium tumefaciens. The T-DNA is transferred from bacterium into the host plant's nuclear DNA genome. The T-DNA is bordered by a right and left border DNA sequence. Transfer is initiated at the right border and terminated at the left border. In plant biotechnology, the tumor-promoting and opine-synthesis genes are removed from the T-DNA and replaced with expression cassettes comprising a gene of interest and/or selection markers, which is required to establish which plants have been successfully transformed. Strains of Agrobacterium used in plant biotechnology comprise vir genes, that were once encoded in the Virulence region of the Ti-plasmid, on a disarmed Ti plasmid which is maintained in the host Agro cell with antibiotic selection. The vir genes are essential in the transfer and insertion of the T-DNA into the plant cell's chromosome. Typically, the plant binary vector plasmid construct used to transform plants in biotechnology comprise a T-DNA which comprises left and right border sequences with transgene expression cassettes between the left and right borders. A plasmid backbone comprises replication origins and antibiotic selection genes necessary to maintain the plasmid in both Escherichia coli and Agrobacterium tumefaciens.

A “transgene” refers to a transcribable DNA molecule heterologous to a host cell at least with respect to its location in the host cell genome and/or a transcribable DNA molecule artificially incorporated into a host cell's genome in the current or any prior generation of the cell.

“Transgenic plant” refers to a plant that comprises within its cells a heterologous polynucleotide. In some embodiments, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extrachromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

“Vector” refers to a polynucleotide or other molecule that transfers nucleic acids between cells. Vectors are often derived from plasmids, bacteriophages, or viruses and optionally comprise parts which mediate vector maintenance and enable its intended use. A “cloning vector” or “shuttle vector” or “subcloning vector” contains operably linked parts that facilitate subcloning steps (e.g., a multiple cloning site containing multiple restriction endonuclease sites). The term “expression vector” as used herein refers to a vector comprising operably linked polynucleotide sequences that facilitate expression of a coding sequence in a particular host organism (e.g., a bacterial expression vector or a plant expression vector).

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The compositions and methods described herein are suitable for use in whole plants, plant parts and plant cells. Plant parts include, but are not limited to, leaves, stems, roots, tubers, seeds, endosperm, ovule, and pollen. Plant parts may be viable, nonviable, regenerable, and/or non-regenerable. Examples of plants which may be mentioned are the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soy beans, wheat, rice, potatoes, cotton, sugar cane, tobacco and oilseed rape.

Also provided herein is a commodity product that is produced from a targeted transposition or part thereof containing the sequence of interest of the donor cassette. Commodity products of the invention contain a detectable amount of DNA comprising a DNA sequence selected from the group consisting of SEQ ID NOs:45-48. As used herein, a “commodity product” refers to any composition or product which is comprised of material derived from a transgenic plant, seed, plant cell, or plant part containing the recombinant DNA molecule of the invention. Commodity products include but are not limited to processed seeds, grains, plant parts, and meal. A commodity product of the invention will contain a detectable amount of DNA corresponding to the transposon cassette. Detection of one or more of this DNA in a sample may be used for determining the content or the source of the commodity product. Any standard method of detection for DNA molecules may be used, including methods of detection disclosed herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. For example, if an item is selected from a group consisting of A, B, C, and D, the inventors specifically envision each alternative individually (e.g., A alone, B alone, etc.), as well as combinations such as A, B, and D; A and C; B and C; etc.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

### Example 1

Anabaena cylindrica gRNA, LE and RE Sequences.

The native sequences of most of the CAST elements have been reported by Strecker et al (2019). However, the crRNA, tracrRNA, LE and RE of the AcCAST system were not reported in that study, and thus bioinformatic methods were used to identify them. Pairwise alignment between the non-coding RNAs of Scytonema hofmanni (Sh) and the corresponding genomic regions of Anabaena cylindrica(Ac) using ClustalW (Thompson et al.; Nucleic Acids Res. 1994; 22(22):4673-4680) was used to identify the putative crRNA and tracrRNA species of Anabaena cylindrica. 500 bp-regions immediately upstream and downstream of the Anabaena cylindrica ActnsB and Cas12k was used to identify the putative AcLE and AcRE sequences. The sequence of the AcsgRNA is disclosed as SEQ ID NO: 55. The AcLE sequence is disclosed as SEQ ID NO:47. The AcRE sequence is disclosed as SEQ ID NO:48.

### Example 2

Transforming Plants with CAST Components Using Agrobacterium tumefaciens

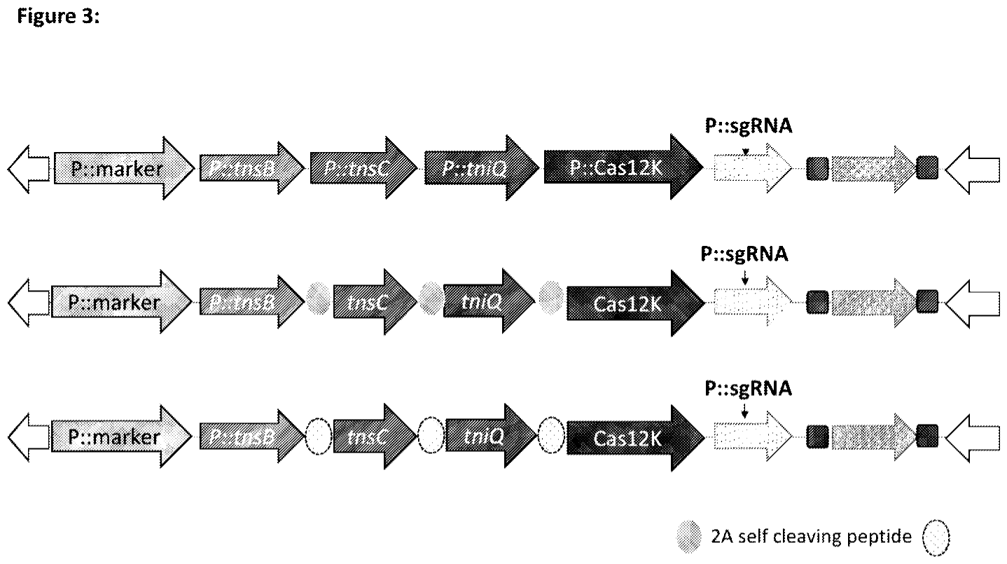

Agrobacterium T-DNA vectors are designed for delivery of CAST system components to plant cells. As shown in FIG. 3A, effector proteins, TnsB, TnsC, TniQ, and Cas12K are encoded by individual gene expression cassettes, which are assembled together in a single T-DNA molecule in a binary vector suitable for use with Agrobacterium tumefaciens strains. As shown in FIG. 3B, sequences encoding the effector proteins of the CAST system are cloned into a T-DNA molecule as a single transcription unit where the TnsB, TnsC, TniQ, and Cas12K encoding sequences are separated by sequences encoding the self-cleaving peptide, 2A, resulting in the production of individual polypeptides corresponding to functional TnsB, TnsC, TniQ, and Cas12K proteins. As shown in FIG. 3C, sequences encoding the effector proteins TnsB, TnsC, TniQ, and Cas12K of the CAST system are cloned into a T-DNA molecule as a single transcription unit where internal ribosome entry sites (IRES) sequences are positioned between the TnsB, TnsC, TniQ, and Cas12K encoding sequences to produce a transcript that results in the production of multiple polypeptides. An expression cassette for a plant selectable marker gene, for example antibiotic resistance or herbicide tolerance is further provided in the T-DNA vectors to aid in selection of transformed plant cells. The T-DNA vectors are further designed to contain an expression cassette for production of at least one suitable gRNA that forms a complex with Cas12k and guides it to hybridize to a target site in a plant genome. The T-DNA vectors also are designed to contain a donor cassette comprising conserved LE and RE elements flanking a nucleic acid sequence of interest.

Gene expression regulatory elements, including, but not limited to, promoters, introns, polyadenylation sequences and transcriptional termination sequences, are chosen to provide suitable expression levels of each expression element on the T-DNA. Gene expression elements that express the gene cassettes at sufficient levels and timing so as to provide all necessary components at the same time and in the same tissue, at levels that are sufficient to result in targeted transposition activity are utilized. Promoters and other regulatory elements may be chosen to provide constitutive gene expression of all the components of the system. Gene expression elements that are diverged from each other at the sequence level in order to reduce the risk of post-transcriptional gene silencing when expressed in coordinated manner may be utilized. The genetic elements included in the T-DNA can be arranged in any order and orientation within T-DNA, but it is preferable to arrange and orient the gene cassettes so as to reduce the possibility of unintended impacts on gene expression. It may be preferable to include insulator or other intervening sequences between some of the gene cassettes.

Transgenic plants containing the T-DNAs described above are selected based on the presence and expression of the selectable marker cassette. Prior to, during, or after the insertion of the T-DNA into the genome, the sequence of interest which is flanked by the LE and RE elements is inserted into the target side determined by the Cas12k and gRNA sequence. This process creates an initial transgenic plant with at least two insertions of transgenic DNA; one or more insertions of all or part of the T-DNA in one or more random locations in the genome, and the donor cassette ‘transposon’ inserted at the desired target site. In the majority of the instances the T-DNA and the donor cassette ‘transposon’ are genetically unlinked, such that, in a subsequent plant generation, the T-DNA and donor cassette can segregate independently of each other, resulting in plants that are devoid of the original T-DNA containing the expression cassettes for the CAST effector proteins.

### Example 3

Optimizing gRNA Function for Cas12k

The gRNA structure and gRNA promoter is optimized to improve CAST activity in plants. To determine how the difference in gRNA expression levels or structure impact Cas12k binding, an assay relying on activating transcription from a minimal promoter upstream of the gene GUS in a reporter construct transfected into corn leaf protoplasts is utilized. Since Cas12k does not cleave DNA, it can be directly modified to encode one NLS domain and a transcription factor domain from a TALE protein (SEQ ID 67) added to the N or C terminal. A reporter construct consisting of the uidA (GUS) reporter gene driven by a minimal CaMV promotor with three adjacent gRNA binding sites will monitor the binding of Cas12k-TALE-TF with expression of the GUS protein indicative of this binding. The Cas12k-TALE-TF with the gRNA can be expressed with or without the CAST system components, tnsB, tnsC, and tniQ, to monitor the efficiency of Cas12k binding in the presence and absence of the other effector proteins of the CAST system. If the Cas12k-TALE-TF can bind and activate transcription in the absence of tnsB, tnsC, tniQ, it may be superior to Cas9 or Cpf1 CRISPR as a backbone to attach transcriptional activators due to Cas12k's smaller size.

Optimization of the promoter for gRNA is undertaken by designing a set of gRNA (based on the sgRNA Strecker et. al. 2019) expression constructs comprising a promotor selected from each class of snRNA genes, namely U6, 7SL, U2, U5, and U3 (see US20170166912A1). When the Cas12k-TALE-TF and gRNA complexes bind the GUS reporter construct, the TALE transcription factor domain will activate the minimal CaMV promoter resulting in higher expression of the GUS transcript, and ultimately higher levels of GUS protein expression. The promoter which provides optimal gRNA expression, as determined by GUS protein expression, will be selected. For some applications of the CAST system, the gRNA promoter which provides the highest levels of GUS expression is selected. In other applications of the CAST system, the gRNA promoter which provides low or moderate levels of GUS expression is selected.

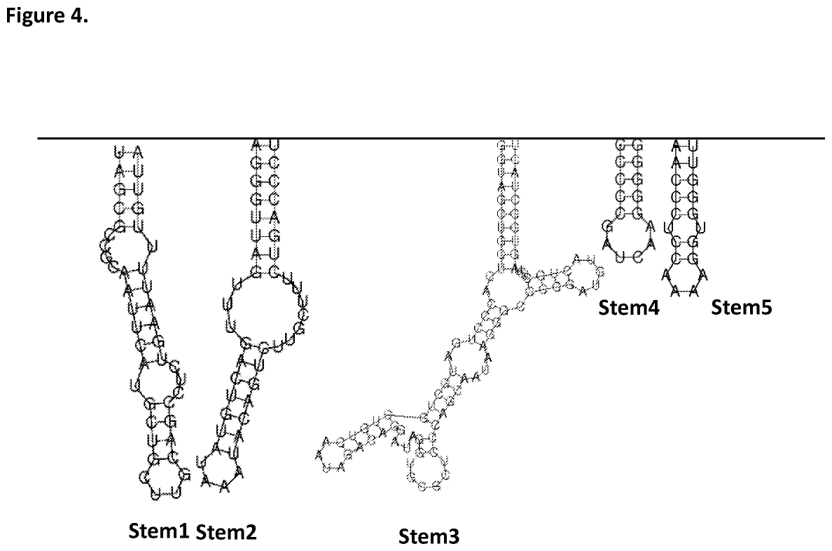

The Cas12k-TALE-TF/GUS reporter system is also used to determine optimal sgRNA sequence and/or structure. Structure of the Cas12k gRNA is optimized using a series of constructs altering the stem size, loop size, bulge size or nucleotide composition of stems 1-5 (see, FIG. 4). The sequence of the Cas12k sgRNA may also be optimized by removing quad or penta mononucleotide stretches by changing sequence, while maintaining structure. The quad T at nucleotides 43-46 could prematurely terminate the sgRNA when expressed under a polIII promoter and the penta C and G of Stem 4 could also impact efficient transcription. Maintaining the structure while altering the nucleotide composition is predicted to increase overall activity. Expression of the Cas12k-TALE-TF and altered sgRNAs complexes with the GUS reporter construct, monitors the efficiency of the Cas12k-TALE-TF/altered sgRNAs complex by the level of activation of the minimal CaMV promotor by the TALE domain, ultimately impacting GUS protein expression. The sgRNA structure which provides optimal Cas12k binding, as determined by GUS protein expression, will be selected. For some applications of the CAST system, the sgRNA sequence and/or structure which provides the highest levels of GUS expression is selected. In other applications of the CAST system, the sgRNA sequence and/or structure which provides low or moderate levels of GUS expression is selected.

### Example 4

Synthetic, Codon-Optimized CAST Sequences for Optimal Expression in Plants and E coli:

The nucleotide sequence of TnsB, TnsC, TniQ and Cas12k genes from ShCAST and AcCAST systems were analyzed and the open reading frames were codon-optimized for optimal expression in plants and bacteria. The codon-optimized (CO) variants are listed in Table 1.

TABLE 1
Codon-optimized(CO) ShCAST and AcCAST sequences.

| SEQ | | Optimized for expression |
|---|---|---|
| ID NO | CAST protein | in plant/bacteria |
| 1 | ShTnsB_pCO1 | plant |
| 2 | ShTnsB_pCO2 | plant |
| 3 | ShTnsC_pCO1 | plant |
| 4 | ShTnsC_pCO2 | plant |
| 5 | ShTniQ_pCO1 | plant |
| 6 | ShTniQ_pCO2 | plant |
| 7 | ShCas12k_pCO1 | plant |
| 8 | ShCAs12k_pCO2 | plant |
| 9 | AcTnsB_pCO1 | plant |
| 10 | AcTnsC_pCO1 | plant |
| 11 | AcTniQ_pCO1 | plant |
| 12 | AcCas12k_pCO1 | plant |
| 13 | ShTnsB_pCO3 | plant |
| 14 | ShTnsB_pCO4 | plant |
| 15 | ShTnsB_pCO5 | plant |
| 16 | ShTnsC_pCO3 | plant |
| 17 | ShTnsC_pCO4 | plant |
| 18 | ShTnsC_pCO5 | plant |
| 19 | ShTniQ_pCO3 | plant |
| 20 | ShTniQ_pCO4 | plant |
| 21 | ShTniQ_pCO5 | plant |
| 22 | ShCas12k_pCO3 | plant |
| 23 | ShCas12k_pCO4 | plant |
| 24 | ShCas12k_pCO5 | plant |
| 25 | AcTnsB_pCO2 | plant |
| 26 | AcTnsB_pCO3 | plant |
| 27 | AcTnsB_pCO4 | plant |
| 28 | AcTnsC_pCO2 | plant |
| 29 | AcTnsC_pCO3 | plant |
| 30 | AcTnsC_pCO4 | plant |
| 31 | AcTniQ_pCO3 | plant |
| 32 | AcTniQ_pCO4 | plant |
| 33 | AcTniQ_pCO5 | plant |
| 34 | AcCas12k_pCO3 | plant |
| 35 | AcCas12k_pCO4 | plant |
| 36 | AcCas12k_pCO5 | plant |
| 37 | ShTnsB_bCO1 | bacteria |
| 38 | ShTnsC_bCO1 | bacteria |
| 39 | ShTniQ_bCO1 | bacteria |
| 40 | ShCas12k_bCO1 | bacteria |
| 41 | AcTnsB_bCO1 | bacteria |
| 42 | AcTnsC_bCO1 | bacteria |
| 43 | AcTniQ_bCO1 | bacteria |
| 44 | AcCas12k_bCO1 | bacteria |

### Example 5

### Assaying CAST Activity in Soy Protoplasts

Plant optimized expression cassettes for CAST proteins: To facilitate nuclear localization of the CAST proteins in soy, sequences encoding a potato nuclear localization signal (NLS) (WO2019084148-81) and a tomato NLS (WO2019084148-82) are incorporated at the 5′ and 3′ termini of the open reading frames of plant codon-optimized Sh/Ac TnsB, TnsC, TniQ and Cas12k genes (SEQ ID NOs 1-36 lacking the last 3 nucleotides coding for the termination codon) described in Table 1. The NLS encoding open reading frames are operably linked to a Medicago truncatula promoter cassette (US20180230479-0031) and a Medicago truncatula transcription terminator sequence (US20180230478-0001) (see FIG. 1A). The expression cassettes are subsequently introduced into suitable plant expression vectors.

Donor/Transposon cassette: ShDonor and AcDonor cassettes comprising the transposon cassette are created for this assay (FIG. 1C). Both cassettes comprise an E. coli adenylyltransferase gene (aadA) fused to a nucleotide sequence encoding a chloroplast targeting peptide and operably linked to Arabidopsis thaliana actin promoter and an Agrobacterium tumefaciens NOS gene terminator sequence. The aadA gene provides resistance against spectinomycin and serves as a selectable marker. The aadA cassette is flanked by the conserved LE and RE elements from the Sh or AcCAST system. ShLE is disclosed as SEQ ID NO:45. ShRE is disclosed as SEQ ID NO:46. The AcDonor cassette is flanked by the conserved LE and RE elements from AcCAST system. AcLE is disclosed as SEQ ID NO:47. AcRE is disclosed as SEQ ID NO:48. The expression cassettes are subsequently introduced into suitable plant expression vectors.

Selection of Target sites in the soy genome: The Phytoene desaturase (GmPDS) gene on Chromosome 18 (GENBANK ACCESSION CM000851) is chosen as the target region for site directed integration of the donor cassette by the ShCAST system. Five GmPDS1 Target sites are chosen based on the occurrence of the appropriate BGTT PAM site at the 5′ end (see Table 2).

TABLE 2
Sequences of soy target sites selected for
ShCAST mediated insertion.

| SEQ | Target site | | |
|---|---|---|---|
| ID NO: | description | 5′PAM | Target site Sequence |
| 49 | GmPDSChr18- | gtt | gctgcatggaaagacaaggatgg |
| | TS1 | | |
| 50 | GmPDSChr18- | gtt | gatccttgacactatcaaagcct |
| | TS2 | | |
| 51 | GmPDSChr18- | gtt | ggtgtatgttcttaggggaagct |
| | TS3 | | |
| 52 | GmPDSChr18- | gtt | gattgtcactcaattcgggaggc |
| | TS4 | | |
| 53 | GmPDSChr18- | gtt | ggcaattcaaaacagcagatctt |
| | TS5 | | |

Single-guide RNA expression cassettes for Soy: Cas12k in its native configuration utilizes both a CRISPR RNA (crRNA) and separate trans-activating CRISPR RNA (tracrRNA). To create a single-guide RNA (sgRNA), the tracrRNA is fused with the crRNA using a pentaloop (GAAAA). Unique ShsgRNA constructs are designed to guide the ShCas12k protein to the selected target sites within GmPDS1. Each sgRNA construct comprises the DNA sequence encoding the tracrRNA sequence, the pentaloop sequence and the crRNA sequence. The crRNA sequence further comprises a repeat sequence and a variable sequence that is complementary to the target site on the soy chromosome (SEQ ID 49 to 53). The sequence of the tracer RNA-pentaloop-repeat sequence for ShsgRNA is set for as SEQ ID NO 54. The sequence of the tracer RNA-pentaloop-repeat sequence for AcsgRNA is set for as SEQ ID NO 55. A ‘G’ nucleotide is added at the 5′ termini of all sgRNAs and the sequences are operably linked to the Soy U6 promoter cassette (WO2019084148-17) and a polyT₈ terminator sequence. The sgRNA expression cassettes are subsequently introduced into suitable plant expression vectors.

Protoplast transformation and assay for Site-specific integration of donor: Set molar ratios of plant expression vectors comprising the codon-optimized ShTnsB, ShTnsC, ShTniQ and ShCas12k cassettes and at least one ShsgRNA as described above are co-delivered into soy protoplasts together with the ShDonor vector using standard polyethylene glycol (PEG) mediated transformation protocols. Following transformation, the protoplasts are incubated in the dark and harvested after 48 hours. Genomic DNA is isolated and assayed for integration of the donor expression cassette into the preselected GmPDS1 target sites. Flank PCR assays similar to those described in WO2019084148 are used to identify putative targeted insertions. The resulting amplicons will also be sequenced to confirm targeted insertion.

### Example 6

### Assaying ShCAST Activity in Soy Plants

An agrobacterium T-DNA vector comprising seven expression cassettes between left border (LB) and right border (RB) sequences is generated. Cassette 1 is an expression cassette for a selectable marker gene aadA. Cassette 2 is an expression cassette comprising the ShTnsB-CO2 sequence (SEQ ID NO:2) fused to the tomato HSFA gene (Heat shock transcription factor) NLS (WO2019084148-0010) at the 5′ end and the 3′ end, operably linked to the Dahlia Mosaic Virus Promoter cassette (WO2019084148, SEQ ID 6-8) and a transcription terminator sequence from Medicago truncatula. Cassette 3 is an expression cassette comprising the ShTnsC-CO2 sequence (SEQ ID NO:4) fused to the tomato HSFA gene (Heat shock transcription factor) NLS (WO2019084148-0010) at the 5′ end and the 3′ end, operably linked to a Cucumis melo Promoter cassette and a transcription terminator sequence from Cotton (US20180216129-0036). Cassette 4 is an expression cassette comprising the ShTniQ-CO2 sequence (SEQ ID NO:6) fused to the tomato HSFA NLS (WO2019084148-0010) at the 5′ end and the 3′ end, operably linked to an Arabidopsis Ubiquitin 10 Promoter cassette and a transcription terminator sequence from cotton (US20180216129-0036). Cassette 5 is an expression cassette comprising the ShCas12k-CO2 sequence (SEQ ID NO: 8) fused to the tomato HSFA NLS at the 5′ end and the 3′ end, operably linked to an Medicago truncatula Ubiquitin 2 Promoter cassette and a transcription terminator sequence also from Medicago truncatula (US20180230478-0001). Cassette 6 is an expression cassette comprising an ShsgRNA targeting at least one Gm.PDS Chr18 target site described in Table 2 and operably linked to a Soybean U6 promoter (WO2019084148-017). Alternatively, the sgRNA cassette is operably linked to a GmU3 promoter (SEQ ID NO 56). Cassette 7 comprises a GUS reporter gene operably linked to a CaMV 35S promoter and an Agrobacterium NOS terminator sequence. The GUS cassette is flanked by the conserved ShLE (SEQ ID NO: 45) and ShRE (SEQ ID NO: 46) transposon sequences.

Excised embryos from A3555 soybean plants are cultured with the Agrobacterium containing the T-DNA vector described above. Transformed plants are selected on selection media, leaf samples from regenerated plantlets are harvested after 4 weeks, and genomic DNA is extracted. The genomic DNA is assayed for integration of the donor expression cassette into the preselected GmPDS1 target site(s). Flank PCR assays will be used to identify putative targeted insertions. The resulting amplicons will also be sequenced to confirm targeted insertion.

### Example 7

### Assaying CAST Activity in Corn Plants

Selection of Target sites in the corn genome: The Zm7 locus (SEQ. ID. NO: 57) is selected as a target region for site-directed integration of a sequence of interest using the CAST system. Based on the occurrence of the appropriate PAM site at the 5′ end, 3 Zm7 target sites are chosen to test the AcCAST system and 6 target sites are chosen for the ShCAST system (see Table 3).

TABLE 3
Sequences of the target sites selected for corn.

| | | | CAST | |
|---|---|---|---|---|
| SEQ | | | system | |
| ID | Target site | | to be | |
| NO: | description | PAM | assayed | Target site Sequence |
| 58 | Zm7_TS1 | AGTG | AcCAST | CTAGCGAGGACAATGAGTCATTC |
| 59 | Zm7_TS2 | AGTG | AcCAST | AGTTGGGAGGACTTGAAAATGTA |
| 60 | Zm7_TS3 | AGTG | AcCAST | TACGGTTCACAGGCAGCCGCCGA |
| 61 | Zm7_TS1 | TGTT | ShCAST | TCAAATGCTGGCCGGCTACTGCC |
| 62 | Zm7_TS2 | TGTT | AcCAST | CTTTATGATAGTCTATTTAGTAT |
| 63 | Zm7_TS3 | TGTT | AcCAST | TATGTTGACAGTGCTAGCGAGGA |
| 64 | Zm7_TS4 | TGTT | AcCAST | ATTTACTGACGTAAGGTATGGTT |
| 65 | Zm7_TS5 | TGTT | AcCAST | GCTTGCTCTTGACAGTGGTGTAC |
| 66 | Zm7_TS6 | TGTT | AcCAST | CACAGGCAGCCGCCGAGAGTGAG |

An agrobacterium T-DNA vector comprising seven expression cassettes is generated. The vector design and composition is similar to the vector described in Example 6 with the exception that the sgRNA cassettes are designed to guide the ShCas12k or AcCas12k protein to the selected target sites within the Zm7 locus described in Table 3. Each sgRNA construct comprises the DNA sequence encoding the tracrRNA sequence, the pentaloop sequence, and the crRNA sequence. The crRNA sequence comprises a repeat sequence and a variable spacer sequence that is complementary to the target site on the chromosome. The sequence of the tracer RNA-pentaloop-repeat sequence for ShsgRNA cassette is set for as SEQ ID NO 30. The sequence of the tracer RNA-pentaloop-repeat sequence for AcsgRNA cassette is set for as SEQ ID NO 31. A ‘G’ nucleotide is added at the 5′ termini of all sgRNAs and the sequences are operably linked to a Maize U6 promoter cassette and a polyT₈ terminator sequence.

Corn embryos are transformed with the Agrobacterium containing a T-DNA vector comprising the expression cassettes described above. Transformed plants are selected on selection media, leaf samples from regenerated plantlets are harvested after 4 weeks, and genomic DNA is extracted. Genomic DNA is isolated and assayed for integration of the donor expression cassette into the preselected Zm7 target site(s). Flank PCR assays will be used to identify putative targeted insertions. The resulting amplicons will also be sequenced to confirm targeted insertion.

1. A method for producing a megalocus on a plant chromosome comprising:
(a) obtaining a plant comprising a first locus, wherein the first locus comprises an endogenous trait locus or is transgenic;
(b) providing to the plant tnsB, tnsC, tniQ, Cas12k, a guide nucleic acid and a donor cassette; and
(c) selecting a progeny plant produced from step (b) wherein targeted transposition of the donor cassette has occurred at a second locus targeted by the guide nucleic acid, wherein the first and second locus are genetically linked but physically separate.

2. The method of claim 1, wherein the first and second locus are located about 0.1 cM to about 20 cM apart from each other.

3. (canceled)

4. The method of claim 1, wherein the plant comprises one or more expression cassettes encoding one or more proteins selected from the group consisting of tnsB, tnsC, tniQ, and Cas12k.

5. The method of claim 1, wherein the plant comprises one or more expression cassettes encoding one or more guide nucleic acids.

6. The method of claim 5, wherein the one or more guide nucleic acids is not complementary to a target site in the plant.

7. The method of claim 1, wherein one or more of tnsB, tnsC, tniQ, Cas12k, a guide nucleic acid and a donor cassette are provided to the plant by particle bombardment.

8. A transgenic plant, seed or plant part comprising a megalocus produced by the method of claim 1.

9. A T-DNA comprising:
a. a first expression cassette encoding a ShTnsB protein comprising a DNA sequence with at least 90%, 95%, 96%, 97%, 98%, 99% or 100% sequence identity to any of SEQ ID NOs: 1, 2, 13-15;
b. a second expression cassette encoding a ShTnsC protein comprising a DNA sequence with at least 90%, 95%, 96%, 97%, 98%, 99% or 100% sequence identity to any of SEQ ID NOs: 3, 4, 16-18; and
c. a third expression cassette encoding a ShTnsQ protein comprising a DNA sequence with at least 90%, 95%, 96%, 97%, 98%, 99% or 100% sequence identity to any of SEQ ID NOs: 5, 6, 19-21.

10-21. (canceled)

22. A T-DNA comprising:
a. a first expression cassette encoding a AcTnsB protein comprising a DNA sequence with at least 90%, 95%, 96%, 97%, 98%, 99% or 100% sequence identity to any of SEQ ID NOs: 9, 25-27;
b. a second expression cassette encoding a AcTnsC protein comprising a DNA sequence with at least 90%, 95%, 96%, 97%, 98%, 99% or 100% sequence identity to any of SEQ ID NOs: 10, 28-30; and
c. a third expression cassette encoding a AcTnsQ protein comprising a DNA sequence with at least 90%, 95%, 96%, 97%, 98%, 99% or 100% sequence identity to any of SEQ ID NOs: 11, 31-33.

23. The T-DNA of claim 22, wherein the T-DNA further comprises a fourth expression cassette encoding a AcCas12k protein comprising a DNA sequence with at least 90%, 95%, 96%, 97%, 98%, 99% or 100% sequence identity to any of SEQ ID NOs: 12, 34-36.

24. The T-DNA of claim 22, wherein the T-DNA further comprises a fifth expression cassette encoding a guide nucleic acid.

25. The T-DNA of claim 24, wherein the expression cassette comprises a DNA sequence with at least 90%, 95%, 96%, 97%, 98%, 99% or 100% sequence identity to SEQ ID NO: 55.

26. A plant comprising the T-DNA of claim 22.

27. The plant of claim 26, wherein the plant further comprises a donor cassette.

28. The plant of claim 27, wherein the donor cassette comprises a DNA sequence with at least 90%, 95%, 96%, 97%, 98%, 99% or 100% sequence identity to SEQ ID NO: 47 and a DNA sequence with at least 90%, 95%, 96%, 97%, 98%, 99% or 100% sequence identity to SEQ ID NO: 48.

29. The T-DNA of claim 22, wherein the T-DNA further comprises a pair of recombinase recognition sequences flanking the expression cassettes encoding CAST system components.

30. The T-DNA of claim 29, wherein the recombinase recognition sequences are selected from the group consisting of LoxP, Lox.TATA-R9, FRT, RS, and GIX.

31. The T-DNA of claim 29, wherein the T-DNA further comprises an expression cassette encoding a site-specific recombinase.

32. The T-DNA of claim 31, wherein the site-specific recombinase is selected from the group consisting of Cre-recombinase, Flp-recombinase, and R-recombinase.

33. The T-DNA of claim 31, wherein the T-DNA further comprises a donor cassette and wherein the donor cassette disrupts the expression cassette encoding the site-specific recombinase.

34. An Agrobacterium tumefaciens bacterium comprising the T-DNA of claim 22.

35. A method of generating a targeted transposition of a sequence of interest in the genome of a plant cell comprising providing to the plant cell a CAST system, wherein the CAST system comprises:
(a) tnsB;
(b) tnsC;
(c) tniQ;
(d) Cas12k;
(e) a guide nucleic acid; and
(f) a donor cassette,
wherein the CAST system transposes the sequence of interest into a target site recognized by the guide nucleic acid in the plant genome.

36. The method of claim 35, wherein the plant cell is produced by crossing a haploid inducer plant to a plant comprising a target site recognized by the guide nucleic acid.

37. The method of claim 35, wherein the plant cell is produced by crossing a first plant comprising (a)-(d) to a second plant comprising (e) and (f).

38. The method of claim 35, wherein the plant cell is produced by bombarding a plant comprising (f) with particles comprising (a)-(e).

39. The method of claim 35, wherein the plant cell is produced by bombarding a plant comprising (a)-(d) with particles comprising (e) and (f).

40. The method of claim 35, wherein the plant comprises a nucleotide sequence encoding any one of (a)-(e) operably linked to a plant-expressible promoter.

41. (canceled)
